(19) United States
(12) Patent Application Publication
(10) Pub. No.: US 2014/0051834 A1
(43) Pub. Date: Feb. 20, 2014

(54) Incretin Receptor Ligand Polypeptide Fc-Region Fusion Polypeptides And Conjugates With Altered Fc-Effector Function

(71) Applicants: Hoffman-La Roche, Inc., Nutley, NJ (US); Indiana University Research and Technology Corporation, Indianapolis, IN (US)

(72) Inventors: Eike HOFFMAN, Herrsching a. Ammersee (DE); Erhard KOPETZKI, Penzberg (DE); Matthias RUETH, Penzberg (DE); Georg TIEFENTHALER, Sindelsdorf (DE); Richard D. DiMARCHI, Carmel, IN (US)

(73) Assignees: Hoffmann-La Roche, Inc., Nutley, NJ (US); Indiana University Research and Technology Corporation, Indianapolis, IN (US)

(21) Appl. No.: 13/920,190

(22) Filed: Jun. 18, 2013

Related U.S. Application Data
(60) Provisional application No. 61/662,576, filed on Jun. 21, 2012.

(51) Int. Cl. C07K 16/46

(52) U.S. Cl. CPC C07K 16/46
USPC 530/387.3

(57) ABSTRACT

Herein is reported an Fc-region fusion polypeptide or Fc-region conjugate comprising one to four incretin receptor ligand polypeptides and a variant human Fc-region with a mutation of the amino acid residue at position 329 and at least one further mutation of at least one amino acid selected from the group comprising amino acid residues at position 228, 233, 234, 235, 236, 237, 297, 318, 320, 322 and 331 to a different residue, wherein the residues in the Fc-region are numbered according to the EU index of Kabat and its use as a medicament.

### CROSS-REFERENCE TO RELATED APPLICATIONS

This application claims priority to U.S. Provisional Patent Application No. 61/662,576, filed Jun. 21, 2012; the contents of which are incorporated by reference in their entirety into the present application.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 76.202 kilobytes ACII (Text) file named “225625_Sequence_Listing.txt” created on Jun. 17, 2013.

### FIELD OF THE INVENTION

Herein are reported fusions and conjugates of incretin receptor ligand polypeptides with an antibody Fc-region, whereby the Fc-region has altered effector function which is effected by one or more amino acid substitutions in the Fc-region compared to a naturally occurring Fc-region.

### BACKGROUND OF THE INVENTION

Monoclonal antibodies have great therapeutic potential and play an important role in today's medical portfolio. During the last decade, a significant trend in the pharmaceutical industry has been the development of monoclonal antibodies (mAbs) and antibody Fc-region fusion polypeptides as therapeutic agents for the treatment of a number of diseases, such as cancers, asthma, arthritis, multiple sclerosis etc.

The Fc-region of an antibody, i.e. the carboxy-terminal regions of the pair of heavy chains of an antibody that comprises the CH3 domain, the CH2 domain, and a portion of the hinge region, has a limited variability and it is involved in at least a part of the physiological effects of antibodies or Fc-region comprising fusion polypeptides or conjugates. The effector functions attributable to the Fc-region of an antibody vary with the class and subclass of the antibody and include e.g. binding of the antibody via its Fc-region to a specific Fc receptor (FcR) on a cell which triggers various biological responses.

For example, formation of the Fc-region/Fc-gamma receptor (Fc/FcγR) complex recruits effector cells to sites of bound antigen, typically resulting in signaling events within the cells and important subsequent immune responses such as release of inflammation mediators, B-cell activation, endocytosis, phagocytosis, or cytotoxic attack. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Ravetch, et al., Annu. Rev. Immunol. 19 (2001) 275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). In addition, an overlapping site on the Fc-region of the molecule also controls the activation of a cell independent cytotoxic function mediated by complement, otherwise known as complement dependent cytotoxicity (CDC).

For the IgG class of Abs, ADCC and ADCP are governed by engagement of the Fc-region with a family of receptors referred to as Fc-gamma (Fcγ) receptors (FcγRs). In humans, this protein family comprises FcγRI (CD64), FcγRII (CD32), including isoforms FcγRIIA, FcγRIIB, and FcγRIIC, and FcγRIII (CD16), including isoforms FcγRIIIA and FcγRIIIB (Raghavan and Bjorkman, Annu. Rev. Cell Dev. Biol. 12 (1996) 181-220; Abes, et al., Expert Reviews (2009) 735-747). FcγRs are expressed on a variety of immune cells, and formation of the Fc/FcγR complex recruits these cells to sites of bound antigen, typically resulting in signaling and subsequent immune responses such as release of inflammation mediators, B-cell activation, endocytosis, phagocytosis, and cytotoxic attack. Furthermore, whereas FcγRI, FcγRIIA/C, and FcγRIIIA are activating receptors characterized by an intracellular immunoreceptor tyrosine-based activation motif (ITAM), FcγRIIB has an inhibitory motif (ITIM) and is therefore inhibitory. Moreover, de Reys, et al., (Blood 81 (1993) 1792-1800) concluded that platelet activation and aggregation induced by monoclonal antibodies, like for example CD9, is initiated by antigen recognition followed by an Fc-region dependent step, which involves the FcγRII-receptor (see also: Taylor, et al., Blood 96 (2000) 4254-4260). While FcγRI binds monomeric IgG with high affinity, FcγRIII and FcγRII are low-affinity receptors, interacting with complexed or aggregated IgG.

The complement inflammatory cascade is a part of the innate immune response and is crucial to the ability for an individual to ward off infection. Another important Fc-region ligand is the complement protein C1q. Fc-region binding to C1q mediates a process called complement dependent cytotoxicity (CDC). C1q is capable of binding six antibodies, although binding to two IgGs is sufficient to activate the complement cascade. C1q forms a complex with the C1r and C1s serine proteases to form the C1 complex of the complement pathway.

In many circumstances, the binding and stimulation of effector functions mediated by the Fc-region of immunoglobulins is highly beneficial, e.g. for a CD20 antibody, however, in certain instances it may be more advantageous to decrease or even to eliminate effector functions. This is particularly true for those antibodies designed to deliver a drug (e.g. toxins or radioisotopes) to the target cell where the Fc/FcγR mediated effector functions bring healthy immune cells into the proximity of the deadly payload, resulting in depletion of normal lymphoid tissue along with the target cells (Hutchins, et al., PNAS USA 92 (1995) 11980-11984; White, et al., Annu. Rev. Med. 52 (2001) 125-145). In these cases the use of antibodies that poorly recruit complement or effector cells would be of a tremendous benefit (see also, Wu, et al., Cell Immunol 200 (2000) 16-26; Shields, et al., J. Biol. Chem. 276 (2001) 6591-6604; U.S. Pat. No. 6,194,551; U.S. Pat. No. 5,885,573 and PCT publication WO 04/029207).

In other instances, for example, where blocking the interaction of a widely expressed receptor with its cognate ligand is the objective, it would be advantageous to decrease or eliminate all antibody effector function to reduce unwanted toxicity. Also, in the instance where a therapeutic antibody exhibited promiscuous binding across a number of human tissues it would be prudent to limit the targeting of effector function to a diverse set of tissues to limit toxicity. Last but not least, reduced affinity of antibodies to the FcγRII receptor in particular would be advantageous for antibodies inducing platelet activation and aggregation via FcγRII receptor binding, which would be a serious side-effect of such antibodies.

Although there are certain subclasses of human immunoglobulins that lack specific effector functions, there are no known naturally occurring immunoglobulins that lack all effector functions completely. An alternate approach would be to engineer or mutate the critical residues in the Fc-region that are responsible for effector function. For examples see WO 2009/100309, WO 2006/076594, WO 1999/58572, US 2006/0134709, WO 2006/047350, WO 2006/053301, U.S. Pat. No. 6,737,056, U.S. Pat. No. 5,624,821, and US 2010/0166740.

The binding of IgG to activating and inhibitory Fcγ receptors or the first component of complement (C1q) depends on residues located in the hinge region and the CH2 domain. Two regions of the CH2 domain are critical for FcγRs and complement C1q binding, and have unique sequences. Substitution of human IgG1 and IgG2 residues at positions 233-236 and IgG4 residues at positions 327, 330 and 331 greatly reduced ADCC and CDC (Armour, et al., Eur. J. Immunol. 29 (1999) 2613-2624; Shields, et al., J. Biol. Chem. 276 (2001) 6591-6604). Idusogie, et al. (J. Immunol 166 (2000) 2571-2575) mapped the C1q binding site for the therapeutic antibody Rituxan® and showed that the Pro329Ala substitution reduced the ability of Rituximab to bind C1q and activate complement. Substitution of Pro329 with Ala has been reported to lead to a reduced binding to the FcγRI, FcγRII and FcγRIIIA receptors (Shields, et al., J. Biol. Chem. 276 (2001) 6591-6604) but this mutation has also been described as exhibiting a wild-type-like binding to the FcγRI and FcγRII and only a very small decrease in binding to the FcγRIIIA receptor (Table 1 and Table 2 in EP 1 068 241, Genentech).

Oganesyan, et al., Acta Cristallographica D64 (2008) 700-704 introduced the triple mutation L234F/L235E/P331S into the lower hinge and C2H domain and showed a decrease in binding activity to human IgG1 molecules to human C1q, FcγRI, FcγRII and FcγRIIIA.

Insulinotropic polypeptides have insulinotropic activity, i.e., have the ability to stimulate, or to cause the stimulation of, the synthesis or expression of the hormone insulin. Insulinotropic peptides include, but are not limited to, GLP-1, exendin-3, exendin-4, and precursors, derivatives, or fragments thereof.

Pro-glucagon-derived peptides, including glucagon and glucagon-like peptide-1 (GLP-1), are found in many metabolic pathways involved in different physiological functions, such as insulin secretion and regulation of food intake.

Pre-pro-glucagon is a 158 amino acid polypeptide that is processed to a number of different active compounds. GLP-1, e.g., corresponds to amino acid residues 72 through 108 of pre-pro-glucagon. GLP-1 among other functions results in the stimulation of insulin synthesis and secretion and inhibition of food intake. GLP-1 has been shown to reduce hyperglycemia (elevated glucose levels) in diabetics.

Glucose-dependent insulinotropic peptide (GIP) is a 42-amino acid gastrointestinal regulatory peptide that stimulates insulin secretion from pancreatic beta cells in the presence of glucose. It is derived by proteolytic processing from a 133-amino acid precursor, pre-pro-GIP.

In WO 2010/011439 GIP-based mixed agonists for treatment of metabolic disorders and obesity are reported. It is reported that modifications to the native glucagon sequence produce glucagon peptides that can exhibit potent glucagon activity equivalent to or better than the activity of native glucagon, potent GIP activity equivalent to or better than the activity of native GIP, and/or potent GLP-1 activity equivalent to or better than the activity of native GLP-1. The data provided is reported to show that peptides having both GIP activity and GLP-1 activity are particularly advantageous for inducing weight loss or preventing weight gain, as well as for treating hyperglycemia, including diabetes, whereby the combination of GIP agonist activity with GLP-1 agonist activity produces a greater effect on weight reduction than GLP-1 alone.

The conjugation of insulinotropic polypeptides to antibodies or antibody fragments is hypothetically outlined in e.g. WO 2010/011439, U.S. Pat. No. 6,329,336 and U.S. Pat. No. 7,153,825.

### SUMMARY OF THE INVENTION

One aspect as reported herein is an Fc-region conjugate comprising one, two, three, or four naturally occurring or synthetic incretin receptor ligand polypeptides each covalently linked to an Fc-region, wherein the conjugate comprises the amino acid sequence LPXTG (SEQ ID NO: 73), where X is optionally an acidic amino acid such as D or E. For example, the amino acid sequence can be LPETG (SEQ ID NO: 74).

It has been found that changing the proline residue at position 329 of an antibody heavy chain Fc-region to glycine results in the inhibition of the FcγRIIIA and FcγRIIA receptor binding and in an inhibition of ADCC and CDC. It has further been found that the combined mutations P329G and for example L234A and L235A (a double point mutation referred to herein as “LALA”) lead to an unexpected strong inhibition of C1q, FcγRI, FcγRIIA and FcγRIIIA Thus, it has been found that a glycine residue in position 329 is unexpectedly advantageous compared to other amino acid substitutions, like alanine.

One aspect as reported herein is an Fc-region fusion polypeptide or Fc-region polypeptide conjugate (also referred to herein as “Fc-region conjugate”) comprising one to four incretin receptor ligand polypeptides and a (variant) human Fc-region, wherein in the Fc-region comprises a mutation of the naturally occurring amino acid residue at position 329 and at least one further mutation of at least one amino acid selected from the group comprising amino acid residues at position 228, 233, 234, 235, 236, 237, 297, 318, 320, 322 and 331 to a different residue, wherein the residues in the Fc-region are numbered according to the EU index of Kabat. The altering of the amino acid residues results in an altering of the effector function of the Fc-region compared to the non-modified (wild-type) Fc-region.

In one embodiment the (variant) human Fc-region of the fusion or conjugate has a reduced affinity to the human FcγRIIIA and/or FcγRIIA and/or FcγRI compared to a fusion polypeptide or conjugate comprising a wild-type IgG Fc-region.

In one embodiment the ADCC induced by the (variant) human Fc-region comprising fusion polypeptide or conjugate is reduced by at least 20% of the ADCC induced by the fusion polypeptide or conjugate comprising a wild-type human IgG Fc-region.

In one embodiment the human Fc-region is a human Fc-region of the human IgG1 isotype or of the human IgG4 isotype.

In one embodiment of the fusion polypeptide or conjugates described herein comprising LPXTG (SEQ ID NO: 75) or LPETG (SEQ ID NO: 74), the amino acid residue at position 329 in the human Fc-region in the fusion polypeptide or conjugate is substituted with glycine, or arginine, or an amino acid residue large enough to destroy the proline sandwich within the Fc-region.

In one embodiment the at least one further mutation of at least one amino acid in the Fc-region is S228P, E233P, L234A, L235A, L235E, N297A, N297D, and/or P331S. In one embodiment the at least one further mutation in the Fc-region is L234A and L235A if the Fc-region is of human IgG1 isotype or S228P and L235E if the Fc-region is of human IgG4 isotype. A double point mutation of S228P and L235E is referred to herein as “SPLE”.

In one embodiment the fusion polypeptide or conjugate has a reduced affinity to at least one further receptor of the group comprising the human FcγI receptor, the human FcγIIA receptor, and C1q, compared to a fusion polypeptide or conjugate comprising a wild-type human IgG Fc-region.

In one embodiment the thrombocyte aggregation induced by the fusion polypeptide or conjugate is reduced compared to the thrombocyte aggregation induced by a fusion polypeptide or conjugate comprising a wild-type human IgG Fc-region.

In one embodiment the fusion polypeptide or conjugate has reduced CDC compared to the CDC induced by a fusion polypeptide or conjugate comprising a wild-type human IgG Fc-region.

In exemplary aspects, the Fc-region of the Fc-region fusion polypeptide or Fc-region polypeptide conjugate comprises the amino acid sequence of any one of SEQ ID NOs: 42-56.

In exemplary aspects, the incretin receptor ligand polypeptide of the Fc-region fusion polypeptide or Fc-region polypeptide conjugate comprises the amino acid sequence of any one of SEQ ID NOs: 1-39, 76, and 77.

In exemplary aspects, the incretin receptor ligand polypeptide is linked to the Fc-region via a linker and the linker comprises the amino acid sequence of any one of SEQ ID NOs: 57-69 and 82-94.

In exemplary aspects, the Fc-region fusion polypeptide or Fc-region polypeptide conjugate comprises the amino acid sequence of YXEGTFTSDYSIYLDKQAAXEFVAWLLAGGPS SGAPPPSKLPETGGGDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSRDELTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 95), wherein X is AIB.

In exemplary aspects, the Fc-region fusion polypeptide or Fc-region polypeptide conjugate comprises the amino acid sequence of YXEGTFTSDYSIYLDKQAAXEFVAWLLAGGGLPETGGGDKTHTCPPCPAPELLGGPSVF LFPPKPKDTLMISRTPEVTCVVVDVSHEDPEVKFNWYVDGVEVHNAKTKPREEQYNST YRVVSVLTVLHQDWLNGKEYKCKVSNKALPAPIEKTISKAKGQPREPQVYTLPPSRDEL TKNQVSLTCLVKGFYPSDIAVEWESNGQPENNYKTTPPVLDSDGSFFLYSKLTVDKSRW QQGNVFSCSVMHEALHNHYTQKSLSLSPGK (SEQ ID NO: 96), wherein X is AIB.

In exemplary aspects, the Fc-region fusion polypeptide or Fc-region polypeptide conjugate comprises the amino acid sequence of YXEGTFTSDYSIYLDKQAAXEFVAWLLAGGPSSGAPPPSKLPETGGGGSGGGGSGGGGS DKTHTCPPCPAPELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 97), wherein X is AIB.

In exemplary aspects, the Fc-region fusion polypeptide or Fc-region polypeptide conjugate comprises the amino acid sequence of YXEGTFTSDYSIYLDKQAAXEEVAWLLAGGGLPETGGGGSGGGGSGGGGSDKTHTCPP CPAPELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQPREP QVYTLPPSRDELTKNQVSLTCLVKGFYPSDIAVEWESNGQPENNYKTTPPVLDSDGSFFL YSKLTVDKSRWQQGNVFSCSVMHEALHNHYTQKSLSLSPGK (SEQ ID NO: 98), wherein X is AIB.

In exemplary aspects, the Fc-region fusion polypeptide or Fc-region polypeptide conjugate is combined with one or more pharmaceutically acceptable carriers. Thus, provided herein are pharmaceutical formulations comprising an Fc-region fusion polypeptide or Fc-region polypeptide conjugate, as described herein, and one or more pharmaceutically acceptable carriers.

One aspect as reported herein is the use of a fusion polypeptide or conjugate as reported herein as a medicament.

One aspect as reported herein is the use of a fusion polypeptide or conjugate as reported herein for treating a disease wherein it is favorable that an effector function of the fusion polypeptide or conjugate is reduced compared to the effector function induced by a fusion polypeptide or conjugate comprising a wild-type human IgG Fc-region.

One aspect as reported herein is the use of a fusion polypeptide or conjugate as reported herein for the manufacture of a medicament for the treatment of a disease, wherein it is favorable that the effector function of the fusion polypeptide or conjugate is reduced compared to the effector function induced by a fusion polypeptide or conjugate comprising a wild-type human IgG Fc-region.

One aspect as reported herein is a method of treating an individual having a disease comprising administering to an individual an effective amount of the fusion polypeptide or conjugate as reported herein, wherein it is favorable that the effector function of the fusion polypeptide or conjugate is reduced compared to the effector function induced by a fusion polypeptide or conjugate comprising a wild-type human Fc-region.

One aspect as reported herein is the use of a fusion polypeptide or conjugate as reported herein for down-modulation of ADCC by at least 20% compared to the ADCC induced by a fusion polypeptide or conjugate comprising a wild-type human IgG Fc-region, and/or for down-modulation of ADCP, wherein Pro329 in the wild-type human IgG Fc-region is substituted with glycine, wherein the residues are numbered according to the EU index of Kabat, and wherein the fusion polypeptide or conjugate exhibits a reduced affinity to the human FcγRIIIA and FcγRIIA.

One aspect as reported herein is the use of a fusion polypeptide or conjugate as reported herein for down-modulation of ADCC by at least 20% compared to the ADCC induced by the polypeptide comprising a wild-type human IgG Fc-region, and/or for down-modulation of ADCP, wherein the Fc-region is of the human IgG class and comprises at least the amino acid substitutions P329G, and L234A and L235A in case of a human IgG1 Fc-region, or S228P and L235E in case of a human IgG4 Fc-region, wherein the residues are numbered according to the EU index of Kabat, wherein the fusion polypeptide or conjugate has a reduced affinity to the human FcγRIIIA and FcγRIIA.

One aspect as reported herein is a method of treating an individual having a disease comprising administering to the individual an effective amount of the fusion polypeptide or conjugate as reported herein, comprising the amino acid sequence LPXTG (SEQ ID NO: 75) or LPETG (SEQ ID NO: 74), wherein Pro329 of the human IgG Fc-region is substituted with glycine, wherein the residues are numbered according to the EU index of Kabat, wherein the fusion polypeptide or conjugate is characterized by a reduced binding to FcγRIIIA and/or FcγRIIA compared to a fusion polypeptide or conjugate comprising a wild-type human IgG Fc-region. In exemplary embodiments, the human IgG Fc-region of such fusion polypeptide or conjugate is a variant of the human IgG1 Fc-region with at least the amino acid substitutions P329G, and L234A and L235A, wherein the residues are numbered according to the EU index of Kabat. In exemplary embodiments, the human IgG Fc-region of such fusion polypeptide or conjugate is a variant of the human IgG4 Fc-region with at least the amino acid substitutions P329G, and S228P and L235E, wherein the residues are numbered according to the EU index of Kabat.

In exemplary aspects, the disease is one described herein in the section entitled “THERAPEUTIC METHODS AND COMPOSITIONS.”

In one embodiment the disease is type-2 diabetes, or insulin resistance.

In one embodiment the disease is obesity.

In one embodiment the disease is type-1 diabetes.

In one embodiment the disease is osteoporosis.

In one embodiment the disease is steatohepatitis, or non-alcoholic fatty liver disease (NAFLD).

In one embodiment, the disease is metabolic syndrome.

In one embodiment the fusion polypeptide or conjugate as reported herein is administered in combination with a further type-2 diabetes drug. In one embodiment the further type-2 diabetes drug is insulin.

In one embodiment the fusion polypeptide or conjugate as reported herein, comprising the amino acid sequence LPXTG (SEQ ID NO: 75) or LPETG (SEQ ID NO: 74), comprises at least two further amino acid substitutions at L234A and L235A (numbered according to the EU index of Kabat) in case of a human IgG1 Fc-region, or S228P and L235E (numbered according to the EU index of Kabat) in case of a human IgG4 Fc-region.

In one embodiment the fusion polypeptide or conjugate as reported herein comprises one incretin receptor ligand polypeptide.

In one embodiment the fusion polypeptide or conjugate as reported herein comprises two incretin receptor ligand polypeptides.

In one embodiment one incretin receptor ligand polypeptide is fused or conjugated to the N-terminus of one Fc-region polypeptide chain.

In one embodiment each of the incretin receptor ligand polypeptides is fused or conjugated to the N-terminus of one Fc-region polypeptide chain, whereby each Fc-region polypeptide chain is fused or conjugated only to one incretin receptor ligand polypeptide.

In one embodiment one incretin receptor ligand polypeptide is fused or conjugated to the C-terminus of one Fc-region polypeptide chain.

In one embodiment each of the incretin receptor ligand polypeptides is fused or conjugated to the C-terminus of one Fc-region polypeptide chain, whereby each Fc-region polypeptide chain is fused or conjugated only to one incretin receptor ligand polypeptide.

In one embodiment one incretin receptor ligand polypeptide is fused or conjugated to an N-terminus of an Fc-region polypeptide chain and one incretin receptor ligand polypeptide is fused or conjugated to the C-terminus of the same or a different Fc-region polypeptide chain.

In one embodiment the two incretin receptor ligand polypeptides are fused to the same Fc-region polypeptide chain.

In one embodiment the two incretin receptor ligand polypeptides are fused to different Fc-region polypeptide chains.

In one embodiment the incretin receptor ligand polypeptide is selected from GIP, GLP-1, exendin-3, exendin-4, dual GIP-GLP-1 agonists, triple GIP-GLP-1-glucagon receptor agonists, chimeric GIP/GLP agonists, and precursors, derivatives, or functional fragments thereof.

In one embodiment the incretin receptor ligand polypeptide is or comprises GLP-1 (7-37) (HAEGTFTSDVSSYLEGQAAKEFIAWLVKGRG, SEQ ID NO: 01), or a precursor, derivative, or fragment thereof that has incretin receptor ligand activity.

In one embodiment the incretin receptor ligand polypeptide is or comprises GLP-1 (7-36) (HAEGTFTSDVSSYLEGQAAKEFIAWLVKGR, SEQ ID NO: 02), or a precursor, derivative, or fragment thereof that has incretin receptor ligand activity.

In one embodiment the incretin receptor ligand polypeptide is or comprises exendin-3 (HSDGTFTSDLSKQMEEEAVRLFIEWLKNGG PSSGAPPPS, SEQ ID NO: 03), or a precursor, derivative, or fragment thereof that has incretin receptor ligand activity.

In one embodiment the incretin receptor ligand polypeptide is or comprises exendin-4 (HGEGTFTSDLSKQMEEEAVRLFIEWLKNGGPSSGAPPPS, SEQ ID NO: 04), or a precursor, derivative, or fragment thereof that has incretin receptor ligand activity.

In accordance with some embodiments of the invention, the incretin receptor ligand polypeptide is a derivative of any of SEQ ID NOs: 01-04 and exhibits incretin receptor ligand activity. In exemplary aspects, the derivative comprises the amino acid sequence of SEQ ID NO: 01 to 04 with 1 to 10 (e.g., 1, 2, 3, 4, 5, 6, 7, 8, 9, 10) amino acid modifications relative to SEQ ID NO: 01-04. In exemplary aspects, the derivative comprises an amino acid sequence which has at least 65% amino acid sequence identity to one of SEQ ID NOs: 01-04. For example, the derivative may comprise an amino acid sequence which has at least 70%, at least 75%, at least 80%, at least 85%, at least 90%, at least 92.5%, at least 95%, at least 97.5%, or more amino acid sequence identity to one of SEQ ID NOs: 01-04.

In one embodiment the incretin receptor ligand polypeptide is or comprises the amino acid sequence exendin-4(1-31) desGlu(17) Tyr(32) (HGEGTFTSDLSKQMEEAVRLFIEWLKNGGPY, SEQ ID NO: 05).

In one embodiment the incretin receptor ligand polypeptide is or comprises the amino acid sequence exendin-4(1-30) Tyr(31) (HGEGTFTSDLSKQMEEEAVRLFIEWLKNGGY, SEQ ID NO: 06).

In one embodiment the incretin receptor ligand polypeptide is or comprises the amino acid sequence exendin-4(9-39) (DLSKQMEEEAVRLFIEWLKNGGPSSGAPPPS, SEQ ID NO: 07).

In one embodiment the incretin receptor ligand polypeptide is or comprises the amino acid sequence SYLEGQAAKEFIAWLVXGR (SEQ ID NO: 08) with X=K or R.

In one embodiment the incretin receptor ligand polypeptide is or comprises the amino acid sequence SSYLEGQAAKEFIAWLVXGR (SEQ ID NO: 09) with X=K or R.

In one embodiment the incretin receptor ligand polypeptide is or comprises the amino acid sequence VSSYLEGQAAKEFIAWLVXGR (SEQ ID NO: 10) with X=K or R.

In one embodiment the incretin receptor ligand polypeptide is or comprises the amino acid sequence DVSSYLEGQAAKEFIAWLVXGR (SEQ ID NO: 11) with X=K or R.

In one embodiment the incretin receptor ligand polypeptide is or comprises the amino acid sequence SDVSSYLEGQAAKEFIAWLVXGR (SEQ ID NO: 12) with X=K or R.

In one embodiment the incretin receptor ligand polypeptide is or comprises the amino acid sequence TSDVSSYLEGQAAKEFIAWLVXGR (SEQ ID NO: 13) with X=K or R.

In one embodiment the incretin receptor ligand polypeptide is or comprises the amino acid sequence FTSDVSSYLEGQAAKEFIAWLVXGR (SEQ ID NO: 14) with X=K or R.

In one embodiment the incretin receptor ligand polypeptide is or comprises the amino acid sequence TFTSDVSSYLEGQAAKEFIAWLVXGR (SEQ ID NO: 15) with X=K or R.

In one embodiment the incretin receptor ligand polypeptide is or comprises the amino acid sequence GTFTSDVSSYLEGQAAKEFIAWLVXGR (SEQ ID NO: 16) with X=K or R.

In one embodiment the incretin receptor ligand polypeptide is or comprises the amino acid sequence EGTFTSDVSSYLEGQAAKEFIAWLVXGR (SEQ ID NO: 17) with X=K or R.

In one embodiment the incretin receptor ligand polypeptide is or comprises the amino acid sequence AEGTFTSDVSSYLEGQAAKEFIAWLVXGR (SEQ ID NO: 18) with X=K or R.

In one embodiment the incretin receptor ligand polypeptide is or comprises the amino acid sequence HAEGTFTSDVSSYLEGQAAKEFIAWLVXGR (SEQ ID NO: 19) with X=K or R.

In one embodiment the incretin receptor ligand polypeptide is or comprises the amino acid sequence HDAEGTFTSDVSSYLEGQAAKEFIAWLVXGR (SEQ ID NO: 20) with X=K or R.

In one embodiment the incretin receptor ligand polypeptide is or comprises the amino acid sequence HAEGTFTSDVSSYLEGQAAKEFIAWLVKGRPSSGAPPPS (SEQ ID NO: 21) (hybrid GLP-1/exendin polypeptide).

In one embodiment the incretin receptor ligand polypeptide is or comprises the amino acid sequence HDEFERHAEGTFTSDVSSYLEGQAAKEFIAWLVKGRK (SEQ ID NO: 22).

In one embodiment the incretin receptor ligand polypeptide is or comprises the amino acid sequence HAEGTFTSDVSSYLEGQAAKEFIAWLVKGRK (SEQ ID NO: 23).

In one embodiment the incretin receptor ligand polypeptide is or comprises the amino acid sequence HGEGTFTSDLSKQMEEEAVRLFIEWLKNGGPSSGAPPPSK (SEQ ID NO: 24).

In one embodiment the incretin receptor ligand polypeptide is or comprises the amino acid sequence HSDGTFTSDLSKQMEEEAVRLFIEWLKNGGPSSGAPPPSK (SEQ ID NO: 25).

In one embodiment the incretin receptor ligand polypeptide is or comprises the amino acid sequence HGEGTFTSDLSKEMEEEVRLFIEWLKNGGPY (SEQ ID NO: 26).

In one embodiment the incretin receptor ligand polypeptide is or comprises the amino acid sequence HGEGTFTSDLSKEMEEEVRLFIEWLKNGGY (SEQ ID NO: 27).

In one embodiment the incretin receptor ligand polypeptide is or comprises the amino acid sequence DLSKQMEEEAVRLFIEWLKGGPSSGPPPS (SEQ ID NO: 28).

In accordance with some embodiments of the invention, the incretin receptor ligand polypeptide is a derivative of native glucagon (SEQ ID NO: 76) and exhibits glucagon receptor ligand activity, GLP-1 receptor ligand activity, and/or GIP receptor ligand activity. In exemplary aspects, the derivative comprises the amino acid sequence of SEQ ID NO: 76 with 1 to 10 (e.g., 1, 2, 3, 4, 5, 6, 7, 8, 9, 10) amino acid modifications relative to SEQ ID NO: 76. In exemplary aspects, the derivative comprises an amino acid sequence which has at least 65% amino acid sequence identity to SEQ ID NO: 76. For example, the derivative may comprise an amino acid sequence which has at least 70%, at least 75%, at least 80%, at least 85%, at least 90%, at least 92.5%, at least 95%, at least 97.5%, or more amino acid sequence identity to SEQ ID NO: 76.

In accordance with some embodiments of the invention, the incretin receptor ligand polypeptide is a derivative of GLP-1 (SEQ ID NO: 1 or 2) and exhibits GLP-1 receptor ligand activity. In exemplary aspects, the derivative comprises the amino acid sequence of SEQ ID NO: 1 or 2 with 1 to 10 (e.g., 1, 2, 3, 4, 5, 6, 7, 8, 9, 10) amino acid modifications relative to SEQ ID NO: 1 or 2, respectively. In exemplary aspects, the derivative comprises an amino acid sequence which has at least 65% amino acid sequence identity to SEQ ID NO: 1 or 2. For example, the derivative may comprise an amino acid sequence which has at least 70%, at least 75%, at least 80%, at least 85%, at least 90%, at least 92.5%, at least 95%, at least 97.5%, or more amino acid sequence identity to SEQ ID NO: 1 or 2.

In accordance with some embodiments of the invention, the incretin receptor ligand polypeptide is a derivative of GIP (SEQ ID NO: 77) and exhibits GIP receptor ligand activity. In exemplary aspects, the derivative comprises the amino acid sequence of SEQ ID NO: 77 with 1 to 10 (e.g., 1, 2, 3, 4, 5, 6, 7, 8, 9, 10) amino acid modifications relative to SEQ ID NO: 77. In exemplary aspects, the derivative comprises an amino acid sequence which has at least 65% amino acid sequence identity to SEQ ID NO: 77. For example, the derivative may comprise an amino acid sequence which has at least 70%, at least 75%, at least 80%, at least 85%, at least 90%, at least 92.5%, at least 95%, at least 97.5%, or more amino acid sequence identity to SEQ ID NO: 77.

In accordance with some embodiments of the invention, the incretin receptor ligand polypeptide is a derivative of exendin-3 or -4 (SEQ ID NO: 3 or 4, respectively) and exhibits exendin ligand activity. In exemplary aspects, the derivative comprises the amino acid sequence of SEQ ID NO: 3 or 4 with 1 to 10 (e.g., 1, 2, 3, 4, 5, 6, 7, 8, 9, 10) amino acid modifications relative to SEQ ID NO: 3 or 4, respectively. In exemplary aspects, the derivative comprises an amino acid sequence which has at least 65% amino acid sequence identity to SEQ ID NO: 3 or 4. For example, the derivative may comprise an amino acid sequence which has at least 70%, at least 75%, at least 80%, at least 85%, at least 90%, at least 92.5%, at least 95%, at least 97.5%, or more amino acid sequence identity to SEQ ID NO: 3 or 4.

In accordance with some embodiments of the invention, the incretin receptor ligand polypeptide is an analog of glucagon (SEQ ID NO: 76) having GIP agonist activity wherein the analog comprises SEQ ID NO: 76 with (a) an amino acid modification at position 1 that confers GIP agonist activity, (b) a modification which stabilizes the alpha helix structure of the C-terminal portion (amino acids 12-29) of the analog, and (c) optionally, 1 to 10 (e.g., 1, 2, 3, 4, 5, 6, 7, 8, 9, 10) further amino acid modifications relative to SEQ ID NO: 76. In some embodiments, the analog exhibits at least about 1% activity of native GIP at the GIP receptor or any other activity level at the GIP receptor described in WO2010/011439. In exemplary aspects, the EC50 of the analog at the GIP receptor is less than about 50-fold different from its EC50 at the GLP-1 receptor.

In certain embodiments, the modification which stabilizes the alpha helix structure is one which provides or introduces an intramolecular bridge, including, for example, a covalent intramolecular bridge, such as any of those described in WO2010/011439. The covalent intramolecular bridge in some embodiments is a lactam bridge. The lactam bridge of the analog of these embodiments can be a lactam bridge as described herein. See, e.g., the teachings of lactam bridges under the section “Stabilization of the Alpha Helix Structure” in WO2010/011439. For example, the lactam bridge may be one which is between the side chains of amino acids at positions i and i+4 or between the side chains of amino acids at positions j and j+3, wherein i is 12, 13, 16, 17, 20 or 24, and wherein j is 17. In certain embodiments, the lactam bridge can be between the amino acids at positions 16 and 20, wherein one of the amino acids at positions 16 and 20 is substituted with Glu and the other of the amino acids at positions 16 and 20 is substituted with Lys.

In alternative embodiments, the modification which stabilizes the alpha helix structure is the introduction of one, two, three, or four α,α-disubstituted amino acids at position(s) 16, 20, 21, and 24 of the analog. In some embodiments, the α,α-disubstituted amino acid is AIB. In certain aspects, the α,α-disubstituted amino acid (e.g., AIB) is at position 20 and the amino acid at position 16 is substituted with a positive-charged amino acid, such as, for example, an amino acid of Formula IV, which is described herein. The amino acid of Formula IV may be homoLys, Lys, Orn, or 2,4-diaminobutyric acid (Dab).

In specific aspects of the invention, the amino acid modification at position 1 is a substitution of His with an amino acid lacking an imidazole side chain, e.g. a large, aromatic amino acid (e.g., Tyr).

In certain aspects, the analog of glucagon comprises amino acid modifications at one, two or all of positions 27, 28 and 29. For example, the Met at position 27 can be substituted with a large aliphatic amino acid, optionally Leu, the Asn at position 28 can be substituted with a small aliphatic amino acid, optionally Ala, the Thr at position 29 can be substituted with a small aliphatic amino acid, optionally Gly, or a combination of two or three of the foregoing. In specific embodiments, the analog of glucagon comprises Leu at position 27, Ala at position 28, and Gly or Thr at position 29.

In certain embodiments of the invention, the analog of glucagon comprises an extension of 1 to 21 amino acids C-terminal to the amino acid at position 29. The extension can comprise the amino acid sequence of GPSSGAPPPS (SEQ ID NO: 78) or XGPSSGAPPPS (SEQ ID NO: 79), for instance. Additionally or alternatively, the analog of glucagon can comprise an extension of which 1-6 amino acids of the extension are positive-charged amino acids. The positive-charged amino acids may be amino acids of Formula IV,

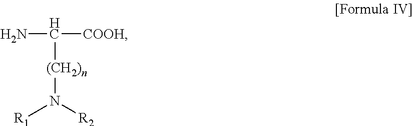

wherein n is 1 to 16, or 1 to 10, or 1 to 7, or 1 to 6, or 2 to 6, each of R₁ and R₂ is independently selected from the group consisting of H, C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl), wherein R₇ is H or OH, and the side chain of the amino acid of Formula IV comprises a free amino group. In exemplary aspects, the amino acid of Formula IV is Lys, homoLys, Orn, or Dab.

Furthermore, in some embodiments, the analog of glucagon (SEQ ID NO: 76) comprises any one or a combination of the following modifications relative to SEQ ID NO: 76:
(a) Ser at position 2 substituted with D-Ser, Ala, D-Ala, Gly, N-methyl-Ser, AIB, Val, or α-amino-N-butyric acid;
(b) Tyr at position 10 substituted with Trp, Om, Glu, Phe, or Val:
(c) Lys at position 12 substituted with Arg or Ile;
(d) Ser at position 16 substituted with Glu, Gln, homoglutamic acid, homocysteic acid, Thr, Gly, or AIB;
(e) Arg at position 17 substituted with Gln;
(f) Arg at position 18 substituted with Ala, Ser, Thr, or Gly;
(g) Gln at position 20 substituted with Ser, Thr, Ala, Lys, Citrulline, Arg, Orn, or AIB;
(h) Asp at position 21 substituted with Glu, homoglutamic acid, homocysteic acid;
(i) Val at position 23 substituted with Ile;
(j) Gln at position 24 substituted with Asn, Ser, Thr, Ala, or AIB;
(k) and a conservative substitution at any of positions 2 5, 9, 10, 11, 12. 13, 14, 15, 16, 8 19 20, 21. 24, 27, 28, and 29.

In exemplary embodiments, the analog of glucagon (SEQ ID NO: 76) having GIP agonist activity comprises the following modifications:
(a) an amino acid modification at position 1 that confers GIP agonist activity,
(b) a lactam bridge between the side chains of amino acids at positions i and i+4 or between the side chains of amino acids at positions j and j+3, wherein i is 12, 13, 16, 17, 20 or 24, and wherein j is 17,
(c) amino acid modifications at one, two or all of positions 27, 28 and 29, e.g., amino acid modifications at position 27 and/or 28, and
(d) 1-9 or 1-6 further amino acid modifications, e.g. 1, 2, 3, 4, 5, 6, 7, 8 or 9 further amino acid modifications, relative to SEQ ID NO: 76
   and the EC50 of the analog for GIP receptor activation is about 10 nM or less. In exemplary aspects, the EC50 of the analog at the GIP receptor is less than about 50-fold different from its EC50 at the GLP-1 receptor.

The lactam bridge of the analog of these embodiments can be a lactam bridge as described herein. See, e.g., the teachings of lactam bridges under the section “Stabilization of the Alpha Helix Structure” in WO2010/011439 For example, the lactam bridge can be between the amino acids at positions 16 and 20, wherein one of the amino acids at positions 16 and 20 is substituted with Glu and the other of the amino acids at positions 16 and 20 is substituted with Lys.

In one embodiment the incretin receptor ligand polypeptide is an analog of glucagon having GIP agonist activity, with the following modifications:
(a) an amino acid modification at position 1 that confers GIP agonist activity,
(b) one, two, three, or all of the amino acids at positions 16, 20, 21, and 24 of the analog is substituted with an α,α-disubstituted amino acid,
(c) amino acid modifications at one, two or all of positions 27, 28 and 29, and
(d) 1-9 or 1-6 further amino acid modifications, e.g. 1, 2, 3, 4, 5, 6, 7, 8 or 9 further amino acid modifications, relative to native glucagon (SEQ ID NO: 76),
   wherein the EC₅₀ of the analog for GIP receptor activation is about 10 nM or less. In exemplary aspects, the EC50 of the analog at the GIP receptor is less than about 50-fold different from its EC50 at the GLP-1 receptor.

The α,α-disubstituted amino acid of the analog of these embodiments can be any α,α-disubstituted amino acid, including, but not limited to, amino iso-butyric acid (AIB), an amino acid disubstituted with the same or a different group selected from methyl, ethyl, propyl, and n-butyl, or with a cyclooctane or cycloheptane (e.g., 1-aminocyclooctane-1-carboxylic acid). In one embodiment the α,α-disubstituted amino acid is aminoisobutyric acid (aib).

In yet other exemplary embodiments, the analog of glucagon (SEQ ID NO: 76) having GIP agonist activity comprises the following modifications:
(a) an amino acid modification at position 1 that confers GIP agonist activity,
(b) an amino acid substitution of Ser at position 16 with an amino acid of Formula IV:

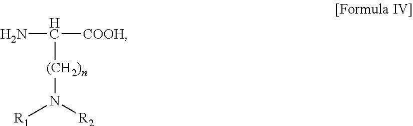

wherein n is 1 to 16, or 1 to 10, or 1 to 7, or 1 to 6, or 2 to 6, each of R₁ and R₂ is independently selected from the group consisting of H, C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl), wherein R₇ is H or OH, and the side chain of the amino acid of Formula IV comprises a free amino group,
(c) an amino acid substitution of the Gln at position 20 with an alpha, alpha-disubstituted amino acid,
(d) amino acid modifications at one, two or all of positions 27, 28 and 29, e.g., amino acid modifications at position 27 and/or 28, and
(e) 1-9 or 1-6 further amino acid modifications, e.g. 1, 2, 3, 4, 5, 6, 7, 8 or 9 further amino acid modifications,
   and the EC50 of the analog for GIP receptor activation is about 10 nM or less. In exemplary aspects, the EC50 of the analog at the GIP receptor is less than about 50-fold different from its EC50 at the GLP-1 receptor.

The amino acid of Formula IV of the analog of these embodiments may be any amino acid, such as, for example, the amino acid of Formula IV, wherein n is 1, 2, 3, 4, 5, 6, 7, 8, 9, 10, 11, 12, 13, 14, 15, or 16. In certain embodiments, n is 2, 3, 4, or 5, in which case, the amino acid is Dab, Orn, Lys, or homoLys respectively.

The alpha, alpha-disubstituted amino acid of the analog of these embodiments may be any alpha, alpha-disubstituted amino acid, including, but not limited to, amino iso-butyric acid (AIB), an amino acid disubstituted with the same or a different group selected from methyl, ethyl, propyl, and n-butyl, or with a cyclooctane or cycloheptane (e.g., 1-aminocyclooctane-1-carboxylic acid). In certain embodiments, the alpha, alpha-disubstituted amino acid is AIB.

In one embodiment the incretin receptor ligand polypeptide is or comprises the amino acid sequence YXEGTFTSDYSIYLDKQAAXEFVCWLLAGGPSSGAPPPSK (SEQ ID NO: 29) with X=aib.

In one embodiment the incretin receptor ligand polypeptide is or comprises the amino acid sequence YXEGTFTSDYSIYLDKQAAXEFVNWLLAGGPSSGAPPPSK (SEQ ID NO: 30) with X=aib.

In one embodiment the incretin receptor ligand polypeptide is or comprises the amino acid sequence YXEGTFTSDYSIYLDKQAAXEFVAWLLAGGPSSGAPPPSK (SEQ ID NO: 31) with X=aib.

In one embodiment the incretin receptor ligand polypeptide is or comprises the amino acid sequence YXEGTFTSDYSIYLDKQAAXEFVNWLLAGGG (SEQ ID NO: 32) with X=aib. One aspect as reported herein is an incretin receptor ligand polypeptide comprises the amino acid sequence YXEGTFTSDYSIYLDKQAAXEFVNWLLAGGG (SEQ ID NO: 32) with X=aib.

In one embodiment the incretin receptor ligand polypeptide is or comprises the amino acid sequence YXEGTFTSDYSIYLDKQAAXEFVAWLLAGG G (SEQ ID NO: 33) with X=aib. One aspect as reported herein is an incretin receptor ligand polypeptide comprises the amino acid sequence YXEGTFTSDYSIYLDKQAAXEFVAWLLAGGG (SEQ ID NO: 33) with X=aib.

In one embodiment the incretin receptor ligand polypeptide is or comprises the amino acid sequence YXEGTFTSDYSIYLDEQAAKEFVNWLLAGGPSSGAPPPSC (SEQ ID NO: 34) with X=aib.

In one embodiment the incretin receptor ligand polypeptide is or comprises the amino acid sequence YXEGTFTSDYSIYLDKQAAXEFVNWLLAGGPSSGAPPPSC (SEQ ID NO: 35) with X=aib.

In one embodiment the incretin receptor ligand polypeptide is or comprises the amino acid sequence YXQGTFTSDYSIYLDKQAAXEFVNWLLAGGPSSGAPPPSK (SEQ ID NO: 36) with X=aib.

In one embodiment the incretin receptor ligand polypeptide is or comprises the amino acid sequence YXQGTFTSDYSIYLDEQAAKEFVNWLLAGGPSSGAPPPSC (SEQ ID NO: 37) with X=aib and with a lactam ring between residues 16 and 20.

In one embodiment the incretin receptor ligand polypeptide is or comprises the amino acid sequence YXQGTFISDYSIYLDEQAAKEFVNWLLAGGPSSGAPPPSC (SEQ ID NO: 38) with X=aib and with a lactam ring between residues 16 and 20.

In one embodiment the incretin receptor ligand polypeptide is or comprises the amino acid sequence YXQGTFISDYSIYLDEQAAKEFVCWLLAG (SEQ ID NO: 39) with X=aib and with a lactam ring between residues 16 and 20.

### DESCRIPTION OF THE FIGURES

FIG. 1

Binding affinities of different FcγRs towards immunoglobulin measured by Surface Plasmon Resonance (SPR) using a BIAcore T100 instrument (GE Healthcare) at 25° C.:
a) FcγRI binding affinity of an anti-CD20 antibody with different variant Fc-regions (IgG1-P329G, IgG4-SPLE and IgG1-LALA) and of an anti-P-selectin antibody with different variant Fc-regions (IgG1-P329G, IgG1-LALA and IgG4-SPLE) as well as for these antibodies comprising a wild-type Fc-region.
b) FcγRI binding affinity of an anti-CD9 antibody with different Fc-regions (IgG1-wild-type, IgG1-P329G, IgG1-LALA, IgG4-SPLE, IgG1-P329G/LALA, IgG4-SPLE/P329G).
c) FcγRIIA binding affinity of an anti-CD9 antibody with different Fc-regions (IgG1-wild-type, IgG1-P329G, IgG1-LALA, IgG4-SPLE, IgG1-P329G/LALA, SPLE/P329G); a normalized response is shown as a function of the concentration of the receptor.
d) FcγRIIB binding affinity of an anti-CD9 antibody with different Fc-regions (IgG1-wild-type, IgG4-SPLE/P329G, IgG1-LALA, IgG1-LALA/P329G) and an anti-P-selectin antibody with different Fc-regions (IgG4-wild-type, IgG4-SPLE).
e) FcγRIIIAV158 binding affinity of an anti-CD9 antibody with different Fc-regions (IgG1-wild-type, IgG4-SPLE, IgG1-LALA, IgG4-SPLE/P329G, IgG1-P329G, IgG1-LALA/P329G); a normalized response is shown as a function of the concentration of the receptor.

FIG. 2

C1q binding of an anti-P-selectin antibody with different Fc-regions (IgG1 wild-type, P329G, IgG4-SPLE) and an anti-CD20 antibody with different Fc-regions (IgG1-wild-type, P329G and IgG4-SPLE).

FIG. 3

Potency to recruit immune-effector cells: Fc-region variants were coated on an ELISA plate and human effector cells transfected with human FcγRIIIA were added. Induction of cytolytic activity of activated NK cells was measured using an esterase assay.
a) an anti-CD20 antibody with different Fc-regions (wild-type, LALA, P329G, P329G/LALA);
b) an anti-CD9 antibody with different Fc-regions (P329R, P329G).

FIG. 4

Potency to recruit immune-effector cells: Human effector cells transfected with human FycRIIIA were used as effectors and CD20 positive Raji cells were used as target cells.
a) non-glycoengineered anti-CD20 antibody with different Fc-regions (P329G, LALA and P329G/LALA);
b) glycoengineered anti-CD20 antibody with different Fc-regions (P329G, P329A and LALA); control: non-glycoengineered anti-CD20 antibody.

FIG. 5

Complement dependent cytotoxicity (CDC) assay: Different antibodies with different Fc-regions were analyzed for their efficiency to mediate CDC on SUDH-L4 target cells.
a) non-glycoengineered anti-CD20 antibody with different Fc-regions (P329G, LALA and P329G/LALA);
b) glycoengineered anti-CD20 antibody with different Fc-regions (P329G, P329A and LALA).

FIG. 6
a) Carbohydrate profile of Fc-associated glycans of human IgG1 variants. The percentage of galactosylation on Fc-associated oligosacchrides of hIgG1 containing the LALA, P329G, P329A or P329G/LALA mutations only differs minimally from that of wild type antibody.
b) Relative galactosylation: Four different IgGs with introduced IgG1 P329G/LALA mutations. Four different V-domains were compared for their amount of galactosylation when expressed in Hek293 EBNA cells.

FIG. 7

Antibody-induced platelet aggregation in whole blood assay. Murine IgG1 induced platelet aggregation as determined for two donors differing in their response in dependence of the antibody concentration.
a) Donor A, b) Donor B.

FIG. 8

SDS-PAGE analysis of sortase-mediated transpeptidation reactions.

FIG. 9

Results of food intake study using different incretin receptor ligand polypeptide formats.

FIG. 10

Course of body weight during food intake study using different incretin receptor ligand polypeptide formats.

### DETAILED DESCRIPTION OF EMBODIMENTS OF THE INVENTION

### I. Definitions

In the present specification and claims the numbering of the residues in an immunoglobulin heavy chain Fc-region is that of the EU index of Kabat (Kabat, E. A., et al., Sequences of Proteins of Immunological Interest, 5th ed., Public Health Service, National Institutes of Health, Bethesda, Md. (1991), NIH Publication 91-3242, expressly incorporated herein by reference). The term “EU index of Kabat” denotes the residue numbering of the human IgG1 EU antibody.

The term “affinity” denotes the strength of the sum total of non-covalent interactions between a single binding site of a molecule (e.g., an antibody) and its binding partner (e.g., an antigen or an Fc receptor). Unless indicated otherwise, as used herein, “binding affinity” refers to intrinsic binding affinity which reflects a 1:1 interaction between members of a binding pair (e.g., antibody/Fc receptor or antibody and antigen). The affinity of a molecule X for its partner Y can generally be represented by the dissociation constant (Kd). Affinity can be measured by common methods known in the art, including those described herein.

The term “alteration” denotes the mutation, addition, or deletion of one or more amino acid residues in a parent amino acid sequence, e.g. of an antibody or fusion polypeptide comprising at least an FcRn binding portion of an Fc-region, to obtain a variant antibody or fusion polypeptide.

The term “amino acid mutation” denotes a modification in the amino acid sequence of a parent amino acid sequence. Exemplary modifications include amino acid substitutions, insertions, and/or deletions. In one embodiment the amino acid mutation is a substitution. The term “amino acid mutations at the position” denotes the substitution or deletion of the specified residue, or the insertion of at least one amino acid residue adjacent the specified residue. The term “insertion adjacent to a specified residue” denotes the insertion within one to two residues thereof. The insertion may be N-terminal or C-terminal to the specified residue.

The term “amino acid substitution” denotes the replacement of at least one amino acid residue in a predetermined parent amino acid sequence with a different “replacement” amino acid residue. The replacement residue or residues may be a “naturally occurring amino acid residue” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). In one embodiment the replacement residue is not cysteine. Substitution with one or more non-naturally occurring amino acid residues is also encompassed by the definition of an amino acid substitution herein. A “non-naturally occurring amino acid residue” denotes a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine, aib and other amino acid residue analogues such as those described in Ellman, et al., Meth. Enzym. 202 (1991) 301-336. To generate such non-naturally occurring amino acid residues, the procedures of Noren, et al. (Science 244 (1989) 182) and/or Ellman, et al. (supra) can be used. Briefly, these procedures involve chemically activating a suppressor tRNA with a non-naturally occurring amino acid residue followed by in vitro transcription and translation of the RNA. Non-naturally occurring amino acids can also be incorporated into peptides via chemical peptide synthesis and subsequent fusion of these peptides with recombinantly produced polypeptides, such as antibodies or antibody fragments.

The term “amino acid insertion” denotes the incorporation of at least one additional amino acid residue into a predetermined parent amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as defined above.

The term “amino acid deletion” denotes the removal of at least one amino acid residue at a predetermined position in an amino acid sequence.

The term “antibody variant” denotes a variant of a wild-type antibody, characterized in that at least one alteration in the amino acid sequence relative to the wild-type amino acid sequence is present in the antibody variant amino acid sequence, e.g. introduced by mutation of one or more amino acid residues in the wild-type antibody.

Within this application whenever an amino acid alteration is mentioned it is a deliberated amino acid alteration and not a random amino acid modification.

The term “antibody-dependent cell-mediated cytotoxicity”, short “ADCC”, denotes a cell-mediated reaction in which non-antigen specific cytotoxic cells that express FcRs (e.g. natural killer cells (NK cells), neutrophils, and macrophages) recognize a target cell by binding to immunoglobulin Fc-region and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9 (1991) 457-492.

The term “antibody-dependent cellular phagocytosis”, short “ADCP”, denotes a process by which antibody-coated cells are internalized, either in whole or in part, by phagocytic immune cells (e.g. macrophages, neutrophils, or dendritic cells) that bind to an immunoglobulin Fc-region.

The term “binding to an Fc receptor” denotes the binding of an Fc-region to an Fc receptor in, for example, a BIAcore® assay (Pharmacia Biosensor AB, Uppsala, Sweden).

In the BIAcore® assay the Fc receptor is bound to a surface and binding of the analyte, e.g. an Fc-region comprising fusion polypeptide or an antibody, is measured by surface plasmon resonance (SPR). The affinity of the binding is defined by the terms ka (association constant: rate constant for the association of the Fc-region fusion polypeptide or conjugate to form an Fc-region/Fc receptor complex), kd (dissociation constant; rate constant for the dissociation of the Fc-region fusion polypeptide or conjugate from an Fc-region/Fc receptor complex), and KD (kd/ka). Alternatively, the binding signal of a SPR sensorgram can be compared directly to the response signal of a reference, with respect to the resonance signal height and the dissociation behaviors.

The term “C1q” denotes a polypeptide that includes a binding site for the Fc-region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The term “incretin receptor ligand polypeptide” denotes a naturally occurring or synthetic polypeptide that binds to the glucagon receptor, or/and the glucagon-like-peptide-I (GLP-1) receptor, or/and glucose-dependent insulinotropic peptide (GIP) receptor, i.e. a molecule that has agonist activity for at least one of these receptors.

In one embodiment the incretin receptor ligand polypeptide binds to the glucose-dependent insulinotropic peptide receptor. In one embodiment the incretin receptor ligand polypeptide binds to the glucose-dependent insulinotropic peptide receptor and to the glucagon-like-peptide-I receptor. In one embodiment the incretin receptor ligand polypeptide binds to the glucose-dependent insulinotropic peptide receptor and to the glucagon-like-peptide-I receptor and to the glucagon receptor.

When blood glucose begins to fall, glucagon, a hormone produced by the pancreas, signals the liver to break down glycogen and release glucose, causing blood glucose levels to rise toward a normal level. GLP-I has different biological activities compared to glucagon. Its actions include stimulation of insulin synthesis and secretion, inhibition of glucagon secretion, and inhibition of food intake. GLP-I has been shown to reduce hyperglycemia (elevated glucose levels) in diabetics. Exendin-4, a peptide from lizard venom that shares about 50% amino acid sequence identity with GLP-I, activates the GLP-I receptor and likewise has been shown to reduce hyperglycemia in diabetics. Glucose-dependent insulinotropic peptide (GIP) is a 42-amino acid gastrointestinal regulatory peptide that stimulates insulin secretion from pancreatic beta cells in the presence of glucose. It is derived by proteolytic processing from a 133-amino acid precursor, preproGIP.

The fusion polypeptide or conjugate as reported herein comprises an incretin receptor ligand that has modifications to the native glucagon sequence that exhibits potent glucagon activity equivalent to or better than the activity of native glucagon, potent GIP activity equivalent to or better than the activity of native GIP, and/or potent GLP-I activity equivalent to or better than the activity of native GLP-I.

The effects of the fusion polypeptide or conjugate reported herein include glucose homeostasis, insulin secretion, gastric emptying, intestinal growth, regulation of food intake. Peptides having both GIP activity and GLP-I activity are particularly advantageous for inducing weight loss or preventing weight gain, as well as for treating hyperglycemia, including diabetes.

Incretin receptor ligand polypeptides include, but are not limited to, GLP-1, exendin-3, exendin-4, and precursors, derivatives, or fragments thereof. Exemplary incretin receptor ligand polypeptides are reported in U.S. Pat. No. 5,574,008, U.S. Pat. No. 5,424,286, U.S. Pat. No. 6,514,500, U.S. Pat. No. 6,821,949, U.S. Pat. No. 6,887,849, U.S. Pat. No. 6,849,714, U.S. Pat. No. 6,329,336, U.S. Pat. No. 6,924,264, WO 2003/103572, U.S. Pat. No. 6,593,295, WO 2011/109784, WO 2010/011439, U.S. Pat. No. 6,329,336 and U.S. Pat. No. 7,153,825.

The term “CH2 domain” denotes the part of an antibody heavy chain polypeptide that extends approximately from EU position 231 to EU position 340 (EU numbering system according to Kabat). In one embodiment a CH2 domain has the amino acid sequence of APELLGGPSVFLEPPKPKDTLMISRTPEVTCVWDVSHEDPEVKFNWYVDGVEVHNAKT KPREEQESTYRWSVLTVLHQDWLNGKEYKCKVSNKALPAPIEKTISKAK (SEQ ID NO: 40). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206.

The term “CH3 domain” denotes the part of an antibody heavy chain polypeptide that extends approximately from EU position 341 to EU position 446. In one embodiment the CH3 domain has the amino acid sequence of GQPREPQVYTLPPSRDELTKNQVSLTCLVKGFYPSDIAVEWESNGQPENNYKTTPPVLD SDGSFFLYSKLTVDKSRWQQGNVFSCSVMHEALHNHYTQKSLSLSPG (SEQ ID NO: 41).

The term “class” of an antibody denotes the type of constant domain or constant region possessed by its heavy chain. There are five major classes of antibodies in humans: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “complement-dependent cytotoxicity”, short “CDC”, denotes a mechanism for inducing cell death in which an Fc-region of a target-bound Fc-region fusion polypeptide or conjugate activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC or ADCP by binding complement receptors (e.g., CR3) on leukocytes.

The term “effector function” denotes those biological activities attributable to the Fc-region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis (ADCP); down regulation of cell surface receptors (e.g. B-cell receptor); and B-cell activation. Such function can be effected by, for example, binding of an Fc-region to an Fc receptor on an immune cell with phagocytic or lytic activity, or by binding of an Fc-region to components of the complement system.

The term “reduced effector function” denotes a reduction of a specific effector function associated with a molecule, like for example ADCC or CDC, in comparison to a control molecule (for example a polypeptide with a wild-type Fc-region) by at least 20%. The term “strongly reduced effector function” denotes a reduction of a specific effector function associated with a molecule, like for example ADCC or CDC, in comparison to a control molecule by at least 50%.

The term “effective amount” of an agent, e.g., a pharmaceutical formulation, denotes an amount effective, at dosages and for periods of time necessary, to achieve the desired therapeutic or prophylactic result.

The term “Fc-region” denotes the C-terminal region of an immunoglobulin. The Fc-region is a dimeric molecule comprising disulfide-linked antibody heavy chain fragments (Fc-region polypeptide chains), optionally comprising one, two, three or more disulfide linkages. An Fc-region can be generated by papain digestion, or IdeS digestion, or trypsin digestion of an intact (full length) antibody or can be produced recombinantly.

The Fc-region obtainable from a full length antibody or immunoglobulin comprises residues 226 (Cys) to the C-terminus of the full length heavy chain and, thus, comprises a part of the hinge region and two or three constant domains, i.e. a CH2 domain, a CH3 domain, and optionally a CH4 domain. It is known from U.S. Pat. No. 5,648,260 and U.S. Pat. No. 5,624,821 that the modification of defined amino acid residues in the Fc-region results in phenotypic effects.

The formation of the dimeric Fc-region comprising two identical or non-identical antibody heavy chain fragments is mediated by the non-covalent dimerization of the comprised CH3 domains (for involved amino acid residues see e.g. Dall'Acqua, Biochem. 37 (1998) 9266-9273). The Fc-region is covalently stabilized by the formation of disulfide bonds in the hinge region (see e.g. Huber, et al., Nature 264 (1976) 415-420; Thies, et al., J. Mol. Biol. 293 (1999) 67-79). The introduction of amino acid residue changes within the CH3 domain in order to disrupt the dimerization of CH3-CH3 domain interactions do not adversely affect the neonatal Fc receptor (FcRn) binding due to the location of the CH3-CH3-domain dimerization involved residues are located on the inner interface of the CH3 domain, whereas the residues involved in Fc-region-FcRn interaction are located on the outside of the CH2-CH3 domain.

The residues associated with effector functions of an Fc-region are located in the hinge region, the CH2, and/or the CH3 domain as determined for a full length antibody molecule. The Fc-region associated/mediated functions are:
(i) antibody-dependent cellular cytotoxicity (ADCC),
(ii) complement (C1q) binding, activation and complement-dependent cytotoxicity (CDC),
(iii) phagocytosis/clearance of antigen-antibody complexes,
(iv) cytokine release in some instances, and
(v) half-life/clearance rate of antibody and antigen-antibody complexes.

The Fc-region associated effector functions are initiated by the interaction of the Fc-region with effector function specific molecules or receptors. Mostly antibodies of the IgG1 isotype can effect receptor activation, whereas antibodies of the IgG2 and IgG4 isotypes do not have effector function or have limited effector function.

The effector function eliciting receptors are the Fc receptor types (and sub-types) FcγRI, FcγRII and FcγRIII. The effector functions associated with an IgG1 isotype can be reduced by introducing specific amino acid changes in the lower hinge region, such as L234A and/or L235A, which are involved in FcγR and C1q binding. Also certain amino acid residues, especially located in the CH2 and/or CH3 domain, are associated with the circulating half-life of an antibody molecule or an Fc-region fusion polypeptide in the blood stream. The circulatory half-life is determined by the binding of the Fc-region to the neonatal Fc receptor (FcRn).

The sialyl residues present on the Fc-region glycostructure are involved in anti-inflammatory mediated activity of the Fc-region (see e.g. Anthony, R. M., et al. Science 320 (2008) 373-376).

The numbering of the amino acid residues in the constant region of an antibody is made according to the EU index of Kabat (Kabat, E. A., et al., Sequences of Proteins of Immunological Interest, 5th ed., Public Health Service, National Institutes of Health, Bethesda, Md. (1991), NIH Publication 91 3242).

The term “Fc-region of human origin” denotes the C-terminal region of an immunoglobulin heavy chain of human origin that contains at least a part of the hinge region, the CH2 domain and the CH3 domain. In one embodiment, a human IgG heavy chain Fc-region extends from about Cys226, or from about Pro230, to the carboxyl-terminus of the heavy chain. However, the C-terminal lysine (Lys447) of the Fc-region may or may not be present.

The term “variant Fc-region” denotes an amino acid sequence which differs from that of a “native” or “wild-type” Fc-region amino acid sequence by virtue of at least one “amino acid alteration/mutation”. In one embodiment the variant Fc-region has at least one amino acid mutation compared to a native Fc-region or to the Fc-region of a parent polypeptide, e.g. from about one to about ten amino acid mutations, and in one embodiment from about one to about five amino acid mutations in a native Fc-region or in the Fc-region of the parent polypeptide. In one embodiment the (variant) Fc-region has at least about 80% homology with a wild-type Fc-region and/or with an Fc-region of a parent polypeptide, and in one embodiment the variant Fc-region has least about 90% homology, in one embodiment the variant Fc-region has at least about 95% homology.

The variant Fc-region as reported herein is defined by the amino acid alterations that are contained. Thus, for example, the term P329G denotes a variant Fc-region with the mutation of proline to glycine at amino acid position 329 relative to the parent (wild-type) Fc-region. The identity of the wild-type amino acid may be unspecified, in which case the aforementioned variant is referred to as 329G. For all positions discussed in the present invention, numbering is according to the EU index. The EU index or EU index as in Kabat or EU numbering scheme refers to the numbering of the EU antibody (Edelman, et al., Proc. Natl. Acad. Sci. USA 63 (1969) 78-85, hereby entirely incorporated by reference.) The alteration can be an addition, deletion, or mutation. The term “mutation” denotes a change to naturally occurring amino acids as well as a change to non-naturally occurring amino acids, see e.g. U.S. Pat. No. 6,586,207, WO 98/48032, WO 03/073238, US 2004/0214988, WO 2005/35727, WO 2005/74524, Chin, J. W., et al., J. Am. Chem. Soc. 124 (2002) 9026-9027; Chin, J. W. and Schultz, P. G., ChemBioChem 11 (2002) 1135-1137; Chin, J. W., et al., PICAS United States of America 99 (2002) 11020-11024; and, Wang, L. and Schultz, P. G., Chem. (2002) 1-10 (all entirely incorporated by reference herein).

A polypeptide chain of a wild-type human Fc-region of the IgG1 isotype has the following amino acid sequence:

(SEQ ID NO: 42)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with the mutations L234A, L235A has the following amino acid sequence:

(SEQ ID NO: 43)
DKTHTCPPCPAPEAAGGPSVFLEPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK.

A polypeptide chain of a variant human Fc-region of the IgG1 isotype with a hole mutation has the following amino acid sequence:

(SEQ ID NO: 44)
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPGK.

A polypeptide chain of a variant human Fc-region of the IgG1 isotype with a knob mutation has the following amino acid sequence:

(SEQ ID NO: 45)
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPCRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK.

A polypeptide chain of a variant human Fc-region of the IgG1 isotype with a L234A, L235A and hole mutation has the following amino acid sequence:

(SEQ ID NO: 46)
DKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPGK.

A polypeptide chain of a variant human Fc-region of the IgG1 isotype with a L234A, L235A and knob mutation has the following amino acid sequence:

(SEQ ID NO: 47)
DKTHTCPPCPAPEAAGGPSVFLEPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPCRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK.

A polypeptide chain of a variant human Fc-region of the IgG1 isotype with a P329G mutation has the following amino acid sequence:

(SEQ ID NO: 48)
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK.

A polypeptide chain of a variant human Fc-region of the IgG1 isotype with a L234A, L235A and P329G mutation has the following amino acid sequence:

(SEQ ID NO: 49)
DKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK.

A polypeptide chain of a variant human Fc-region of the IgG1 isotype with a P329G and hole mutation has the following amino acid sequence:

(SEQ ID NO: 50)
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPGK.

A polypeptide chain of a variant human Fc-region of the IgG1 isotype with a P329G and knob mutation has the following amino acid sequence:

(SEQ ID NO: 51)
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVYTLPPCRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK.

A polypeptide chain of a variant human Fc-region of the IgG1 isotype with a L234A, L235A, P329G and hole mutation has the following amino acid sequence:

(SEQ ID NO: 52)
DKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVCTLPPSRDELTKNQVSLSCAVKGFYPSDIAVEWESNGQPENNYKTTPP
VLDSDGSFFLVSKLTVDKSRWQQGNVFSCSVMHEALHNHYTQKSLSLSPGK.

A polypeptide chain of a variant human Fc-region of the IgG1 isotype with a L234A, L235A, P329G and knob mutation has the following amino acid sequence:

(SEQ ID NO: 53)
DKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALGAPIEKTIS
KAKGQPREPQVYTLPPCRDELTKNQVSLWCLVKGFYPSDIAVEWESNGQPENNYKTTP
PVLDSDGSFFLYSKLTVDKSRWQQGNVFSCSVMHEALHNHYTQKSLSLSPGK.

A polypeptide chain of a wild-type human Fc-region of the IgG4 isotype has the following amino acid sequence:

(SEQ ID NO: 54)
ESKYGPPCPSCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK.

A polypeptide chain of a variant human Fc-region of the IgG4 isotype with a S228P and L235E mutation has the following amino acid sequence:

(SEQ ID NO: 55)
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK.

A polypeptide chain of a variant human Fc-region of the IgG4 isotype with a S228P, L235E and P329G mutation has the following amino acid sequence:

(SEQ ID NO: 56)
ESKYGPPCPPCPAPEFEGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLGSSIEKTI
SKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK.

The term “Fc receptor”, short “FcR”, denotes a receptor that binds to an Fc-region. In one embodiment the FcR is a native sequence human FcR. Moreover, in one embodiment the FcR is an FcR which binds an IgG antibody (an Fc gamma receptor) and includes receptors of the FcγRI, FcγRII, and FcγRIII subclasses, including allelic variants and alternatively spliced forms thereof. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (see e.g. Daëron, M., Annu. Rev. Immunol. 15 (1997) 203-234). FcRs are reviewed in Ravetch and Kinet, Annu. Rev. Immunol 9 (1991) 457-492, Capel, et al., Immunomethods 4 (1994) 25-34, de Haas, et al., J. Lab. Clin. Med. 126 (1995) 330-341. Other FcRs, including those to be identified in the future, are encompassed by the term “FcR” herein. The term also includes the neonatal receptor, FcRn, which is responsible for the transfer of maternal IgGs to the fetus (see e.g. Guyer, et al., J. Immunol. 117 (1976) 587; Kim, et al., J. Immunol. 24 (1994) 249).

The term “IgG Fc ligand” denotes a molecule, in one embodiment a polypeptide, from any organism that binds to the Fc-region of an IgG antibody to form an Fc-region/Fc ligand complex. Fc ligands include but are not limited to FcγRs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (see e.g. Davis, et al., Immunological Reviews 190 (2002) 123-136, entirely incorporated by reference). Fc ligands may include undiscovered molecules that bind Fc. In one embodiment IgG Fc ligands are the FcRn and Fc gamma receptors

The term “Fc gamma receptor”, short “FcγR”, denotes any member of the family of proteins that bind the IgG antibody Fc-region and is encoded by an FcγR gene. In humans this family includes but is not limited to FcγRI (CD64), including isoforms FcγRIA, FcγRIB, and FcγRIC, FcγRII (CD32), including isoforms FcγRIIA (including allotypes H131 and R131), FcγRIIB (including FcγRIIB-1 and FcγRIIB-2), and FcγRIIC, and FcγRIII (CD16), including isoforms FcγRIIIA (including allotypes V158 and F158) and FcγRIIIB (including allotypes FcγRIIB-NA1 and FcγRIIB-NA2) (see e.g. Jefferis, et al., Immunol. Lett. 82 (2002) 57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes. The Fc-region-FcγR interaction involved amino acid residues are 234-239 (lower hinge region), 265-269 (B/C loop), 297-299 (D/E loop), and 327-332 (F/G) loop (Sondermann, et al., Nature 406 (2000) 267-273). Amino acid mutations that result in a decreased binding/affinity for the FcγRI, FcγRIIA, FcγRIIB, and/or FcγRIIIA include N297A (concomitantly with a decreased immunogenicity and prolonged half-life binding/affinity) (Routledge, et al., Transplantation 60 (1995) 847; Friend, et al., Transplantation 68 (1999) 1632; Shields, et al., J. Biol. Chem. 276 (1995) 6591-6604), residues 233-236 (Ward and Ghetie, Ther. Immunol. 2 (1995) 77; Armour, et al., Eur. J. Immunol. 29 (1999) 2613-2624). Some exemplary amino acid substitutions are described in U.S. Pat. No. 7,355,008 and U.S. Pat. No. 7,381,408.

The term “neonatal Fc Receptor”, short “FcRn”, denotes a protein that binds the IgG antibody Fc-region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. The interacting amino acid residues of the Fc-region with the FcRn are near the junction of the CH2 and CH3 domains. The Fc-region-FcRn contact residues are all within a single IgG heavy chain. The involved amino acid residues are 248, 250-257, 272, 285, 288, 290-291, 308-311, and 314 (all in the CH2 domain) and amino acid residues 385-387, 428, and 433-436 (all in the CH3 domain) Amino acid mutations that result in an increased binding/affinity for the FcRn include T256A, T307A, E380A, and N434A (Shields, et al., J. Biol. Chem. 276 (2001) 6591-6604).

The terms “wild-type polypeptide” or “parent polypeptide” denote a starting polypeptide, either unmodified (wild-type polypeptide) or already containing at least one alteration distinguishing it from the wild-type (parent polypeptide), which is subsequently altered to generate a variant. The term “wild-type polypeptide” denotes the polypeptide itself, compositions that comprise the polypeptide, or the nucleic acid sequence that encodes it. Accordingly, the term “wild-type Fc-region polypeptide or conjugate” denotes an Fc-region fusion polypeptide or conjugate comprising a naturally occurring Fc-region which is altered to generate a variant.

The term “full length antibody” denotes an antibody having that has a structure and amino acid sequence substantially identical to a native antibody structure as well as polypeptides that comprise the Fc-region as reported herein.

The term “hinge region” denotes the part of an antibody heavy chain polypeptide that joins the CH1 domain and the CH2 domain, e.g. from about position 216 to position about 230 according to the EU number system of Kabat. The hinge regions of other IgG isotypes can be determined by aligning with the hinge-region cysteine residues of the IgG1 isotype sequence.

The hinge region is normally a dimeric molecule consisting of two polypeptides with identical amino acid sequence. The hinge region generally comprises about 25 amino acid residues and is flexible allowing the antigen binding regions to move independently. The hinge region can be subdivided into three domains: the upper, the middle, and the lower hinge domain (see e.g. Roux, et al., J. Immunol. 161 (1998) 4083).

The term “lower hinge region” of an Fc-region denotes the stretch of amino acid residues immediately C-terminal to the hinge region, i.e. residues 233 to 239 of the Fc-region according to the EU numbering of Kabat.

The term “wild-type Fc-region” denotes an amino acid sequence identical to the amino acid sequence of an Fc-region found in nature. Wild-type human Fc-regions include a native human IgG1 Fc-region (non-A and A allotypes), native human IgG2 Fc-region, native human IgG3 Fc-region, and native human IgG4 Fc-region as well as naturally occurring variants thereof.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “position” denotes the location of an amino acid residue in the amino acid sequence of a polypeptide. Positions may be numbered sequentially, or according to an established format, for example the EU index of Kabat for antibody numbering.

The term “treatment” (and grammatical variations thereof such as “treat” or “treating”) denotes a clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variant” denotes a polypeptide which has an amino acid sequence that differs from the amino acid sequence of a parent polypeptide. Typically such molecules have one or more alterations, insertions, or deletions. In one embodiment the variant amino acid sequence has less than 100% sequence identity with the parent amino acid sequence. In one embodiment the variant amino acid sequence has an amino acid sequence from about 75% to less than 100% amino acid sequence identity with the amino acid sequence of the parent polypeptide. In one embodiment the variant amino acid sequence has from about 80% to less than 100%, in one embodiment from about 85% to less than 100%, in one embodiment from about 90% to less than 100%, and in one embodiment from about 95% to less than 100% amino acid sequence identity with the amino acid sequence of the parent polypeptide.

The term “altered” FcR binding affinity or ADCC activity denotes a polypeptide that has either enhanced or diminished FcR binding activity and/or ADCC activity compared to a parent polypeptide (e.g. a polypeptide comprising a wild-type Fc-region). The variant polypeptide which “has increased binding” to an FcR binds at least one FcR with lower dissociation constant (i.e. better/higher affinity) than the parent or wild-type polypeptide. The polypeptide variant which “has decreased binding” to an FcR, binds at least one FcR with higher dissociation constant (i.e. worse/lower affinity) than the parent or a wild-type polypeptide. Such variants which display decreased binding to an FcR may possess little or no appreciable binding to an FcR, e.g., 0-20% binding to the FcR compared to a wild-type or parent IgG Fc-region, e.g. as determined in the Examples described herein.

The polypeptide which binds an FcR with “reduced affinity” in comparison with a parent or wild-type polypeptide, is a polypeptide which binds any one or more of the above identified FcRs with (substantially) reduced binding affinity compared to the parent polypeptide, when the amounts of polypeptide variant and parent polypeptide in the binding assay are (essentially) about the same. For example, the polypeptide variant with reduced FcR binding affinity may display from about 1.15 fold to about 100 fold, e.g. from about 1.2 fold to about 50 fold reduction in FcR binding affinity compared to the parent polypeptide, where FcR binding affinity is determined, for example, as disclosed in the examples disclosed herein.

The polypeptide comprising a variant Fc-region which “mediates antibody-dependent cell-mediated cytotoxicity (ADCC) in the presence of human effector cells less effectively” than a parent polypeptide is one which in vitro or in vivo is (substantially) less effective at mediating ADCC, when the amounts of variant polypeptide and parent polypeptide used in the assay are (essentially) about the same. Generally, such variants will be identified using the in vitro ADCC assay as disclosed herein, but other assays or methods for determining ADCC activity, e.g. in an animal model etc., are contemplated. In one embodiment the variant is from about 1.5 fold to about 100 fold, e.g. from about two fold to about fifty fold, less effective at mediating ADCC than the parent, e.g. in the in vitro assay disclosed herein.

The term “receptor” denotes a polypeptide capable of binding at least one ligand. In one embodiment the receptor is a cell-surface receptor having an extracellular ligand-binding domain and, optionally, other domains (e.g. transmembrane domain, intracellular domain and/or membrane anchor). The receptor to be evaluated in the assay described herein may be an intact receptor or a fragment or derivative thereof (e.g. a fusion protein comprising the binding domain of the receptor fused to one or more heterologous polypeptides). Moreover, the receptor to be evaluated for its binding properties may be present in a cell or isolated and optionally coated on an assay plate or some other solid phase.

The term “receptor binding domain” denotes any native ligand for a receptor, including cell adhesion molecules, or any region or derivative of such native ligand retaining at least a qualitative receptor binding ability of a corresponding native ligand. This definition, among others, specifically includes binding sequences from ligands for the above-mentioned receptors.

### II. Fc-Region Fusion Polypeptide or Conjugate

Herein is reported an Fc-region fusion polypeptide or conjugate comprising a variant Fc-region. The parent polypeptide may, however, be any polypeptide comprising an Fc-region.

The invention is based, in part, on the finding that the combination of two mutations at defined positions in the Fc-region of an Fc-region comprising fusion polypeptide or conjugate results in a complete reduction of the Fc-region associated effector function.

The selection of an effector function eliciting Fc-region is dependent on the intended use of the Fc-region fusion polypeptide or conjugate.

If the desired use is the functional neutralization of a soluble target a non-effector function eliciting isotype or variant should be selected.

If the desired use is the removal of a target an effector function eliciting isotype or variant should be selected.

If the desired use is the antagonization of a cell-bound target a non-effector function eliciting isotype or variant should be selected.

If the desired use is the removal of a target presenting cell an effector function eliciting isotype or variant should be selected.

The circulating half-life of an Fc-region fusion polypeptide or conjugate can be influenced by modulating the Fc-region-FcRn interaction. This can be achieved by changing specific amino acid residues in the Fc-region (Dall'Acqua, W. F., et al., J. Biol. Chem. 281 (2006) 23514-23524; Petkova, S. B., et al., Internat. Immunol. 18 (2006) 1759-1769; Vaccaro, C., et al. Proc. Natl. Acad. Sci. 103 (2007) 18709-18714).

The minimization or even removal of antibody-dependent cell-mediated cytotoxicity (ADCC) and complement-dependent cytotoxicity (CDC) can be achieved by so called hinge-region amino acid changes/substitutions. The amino acid residues chosen for substitution are those expected to be involved in the binding of the Fc-region to human Fc receptors (but not FcRn). This/these amino acid residue changes result in an improved safety profile compared to Fc-region fusion polypeptides or conjugates comprising a wild-type IgG Fc-region.

The classical complement cascade is initiated by the binding and activation of C1q by antigen/IgG immune complexes. This activation results in inflammatory and/or immunoregulatory responses. The minimization or even removal of the activation of the classical complement cascade can be achieved by so called hinge-region amino acid changes/substitutions. The amino acid residues chosen for substitution are those expected to be involved in the binding of the Fc-region to component C1q. One exemplary Fc-region variant with reduced or even eliminated C1q binding is the Fc-region variant comprising the mutations L234A and L235A (LALA).

The binding of an Fc-region fusion polypeptide or conjugate to the neonatal receptor (FcRn) results in the transport of the polypeptide across the placenta and affects the circulatory half-life of the Fc-region fusion polypeptide or conjugate. An increase of the circulatory half-life of an Fc-region fusion polypeptide or conjugate results in an improved efficacy, a reduced dose or frequency of administration, or an improved localization to the target. A reduction of the circulatory half-life of an Fc-region fusion polypeptide or conjugate results in a reduced whole body exposure or an improved target-to-non-target binding ratio.

The amino acid residues required for FcRn binding that are conserved across species are the histidine residues at position 310 and 435 in the Fc-region. These residues are responsible for the pH dependence of the Fc-region FcRn interaction (see, e.g., Victor, G., et al., Nature Biotechnol. 15 (1997) 637-640); Dall'Acqua, W. F., et al. J. Immunol. 169 (2002) 5171-5180). Fc-region mutations that attenuate interaction with FcRn can reduce antibody half-life.

Generally, the Fc-region of the parent Fc-region fusion polypeptide or conjugate comprises an Fc-region, either a wild-type or altered Fc-region. In one embodiment the Fc-region is an Fc-region of human origin. However, the Fc-region of the parent Fc-region fusion polypeptide or conjugate may already have one or more amino acid sequence alterations compared to a wild-type Fc-region. For example, the C1q or FcγR binding activity of the parent Fc-region may have been altered (other types of Fc-region modifications are described in more detail below). In one embodiment the parent Fc-region is “conceptual” and, while it does not physically exist, the antibody engineer may decide upon a variant Fc-region to be used.

In one embodiment the nucleic acid encoding the parent Fc-region fusion polypeptide or parts of the Fc-region polypeptide conjugate is altered to generate a variant nucleic acid sequence encoding the variant Fc-region fusion polypeptide or part of the Fc-region conjugate.

The nucleic acid encoding the amino acid sequence of the variant Fc-region fusion polypeptide or part of the Fc-region conjugate can be prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the Fc-region fusion polypeptide, or can be generated chemically by DNA synthesis.

Site-directed mutagenesis is a suitable method for preparing substitution variants. This technique is well known in the art (see, e.g., Carter, et al., Nucl. Acids Res. 13 (1985) 4431-4443, Kunkel, et al., Proc. Natl. Acad. Sci. USA 82 (1985) 488). Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting polypeptide (see e.g. Higuchi, in PCR Protocols, Academic Press (1990) pp. 177-183, Vallette, et al., Nucl. Acids Res. 17 (1989) 723-733). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells, et al., in Gene 34 (1985) 315-323.

One aspect as reported herein is an Fc-region fusion polypeptide or conjugate comprising an Fc-region of an antibody, in one embodiment of a human antibody, in which at least one amino acid residue has been altered by addition, mutation, or deletion, resulting in reduced or ablated affinity of the Fc-region fusion polypeptide or conjugate for at least one Fc receptor compared to an Fc-region fusion polypeptide or conjugate comprising the parent or wild-type Fc-region.

The Fc-region interacts with a number of receptors or ligands including but not limited to Fc receptors (e.g. FcγRI, FcγRIIA, FcγRIIIA), the complement protein C1q, and other molecules such as proteins A and G. These interactions are essential for a variety of effector functions and downstream signaling events including, but not limited to, antibody dependent cell-mediated cytotoxicity (ADCC), antibody dependent cellular phagocytosis (ADCP) and complement dependent cytotoxicity (CDC).

In one embodiment the Fc-region fusion polypeptide or conjugate as reported herein comprises an Fc-region that has reduced or ablated affinity for an Fc receptor, which can elicit an effector function, compared to an Fc-region fusion polypeptide or conjugate that comprises a parent or wild-type Fc-region, wherein the amino acid sequence of the Fc-region fusion polypeptide or conjugates differs from the amino acid sequence of the parent Fc-region fusion polypeptide or conjugate by at least one addition, mutation, or deletion of at least one amino acid residue.

In one embodiment the Fc-region fusion polypeptide or conjugate as reported herein has at least one or more of the following properties: reduced or ablated effector function (ADCC and/or CDC and/or ADCP), reduced or ablated binding to Fc receptors, reduced or ablated binding to C1q, or reduced or ablated toxicity.

In one embodiment the Fc-region fusion polypeptide or conjugate as reported herein comprises an Fc-region that has at least a mutation or deletion of the proline amino acid residue at position 329 according to the EU index of Kabat.

If one amino acid residue is deleted from an amino acid sequence the remaining amino acid residues maintain their EU-index number although the actual position in the amino acid sequences changes in order to allow the precise identification of specific amino acid residues in multiply mutated Fc-regions.

In one embodiment the Fc-region fusion polypeptides or conjugate comprises a wild-type human Fc-region with an amino acid mutation at position 329 according to the EU index of Kabat. In one embodiment the Fc-region comprises at least one further amino acid mutation.

In one embodiment the Fc-region fusion polypeptide or conjugate comprises a wild-type human Fc-region that has an amino acid substitution, deletion or addition which reduces or diminishes the function of the proline sandwich in the Fc-region.

In one embodiment the proline residue at amino acid position 329 in the Fc-region is mutated to an amino acid residue which is either smaller or larger than proline. In one embodiment the amino acid residue is mutated to glycine, alanine or arginine. In one embodiment the amino acid residue proline at position 329 according to the EU index of Kabat in the Fc-region is mutated to glycine.

In one embodiment the Fc-region fusion polypeptide or conjugate as reported herein comprises a wild-type Fc-region that has at least two amino acid mutations, additions, or deletions.

In one embodiment the Fc-region fusion polypeptide or conjugate as reported herein has a reduced affinity to a human Fc receptor (FcγR) and/or a human complement receptor compared to an Fc-region fusion polypeptide or conjugate comprising a wild-type human Fc-region.

In one embodiment the Fc-region fusion polypeptide or conjugate as reported herein comprises an Fc-region that has a reduced affinity to a human Fc receptor (FcγR) and/or human complement receptor compared to an Fc-region fusion polypeptide or conjugate comprising a wild-type human Fc-region.

In one embodiment the affinity of the Fc-region in the fusion polypeptide or conjugate to at least one of FcγRI, FcγRII, and/or FcγRIIIA is reduced. In one embodiment the affinity to FcγRI and FcγRIIIA is reduced. In one embodiment the affinity to FcγRI, FcγRII and FcγRIIIA is reduced.

In one embodiment the affinity to FcγRI, FcγRIIIA and C1q is reduced.

In one embodiment the affinity to FcγRI, FcγRII, FcγRIIIA and C1q is reduced.

In one embodiment the ADCC induced by the Fc-region fusion polypeptide or conjugate as reported herein is reduced compared to an Fc-region fusion polypeptide or conjugate comprising a wild-type Fc-region. In one embodiment the ADCC is reduced by at least 20% compared to the ADCC induced by an Fc-region fusion polypeptide or conjugate comprising a wild-type Fc-region.

In one embodiment the ADCC and CDC induced by the Fc-region fusion polypeptide or conjugate comprising a wild-type Fc-region is decreased or ablated.

In one embodiment the Fc-region fusion polypeptide or conjugate as reported herein has a decreased ADCC, CDC, and ADCP compared to an Fc-region fusion polypeptide or conjugate comprising a wild-type Fc-region.

In one embodiment the Fc-region fusion polypeptide or conjugate as reported herein comprises at least one amino acid substitution in the Fc-region that is selected from the group comprising S228P, E233P, L234A, L235A, L235E, N297A, N297D, and P331S.

In one embodiment the wild-type Fc-region is a human IgG1 Fc-region or a human IgG4 Fc-region.

In one embodiment the Fc-region fusion polypeptide or conjugate comprises besides the mutation of the amino acid residue proline at position 329 at least one further addition, mutations, or deletion of an amino acid residue in the Fc-region that is correlated with increased stability of the fusion polypeptide or conjugate.

In one embodiment the affinity of the Fc-region fusion polypeptide or conjugate to an FcR is at most 10 to 20% of the affinity of an Fc-region fusion polypeptide or conjugate comprising a wild-type Fc-region.

In one embodiment, the further addition, mutation, or deletion of an amino acid residue in the Fc-region fusion polypeptide or conjugate as reported herein is at position 228 and/or 235 of the Fc-region if the Fc-region is of IgG4 isotype. In one embodiment the amino acid residue serine at position 228 and/or the amino acid residue leucine at position 235 is/are substituted by another amino acid. In one embodiment the Fc-region fusion polypeptide or conjugate comprises a proline residue at position 228 (mutation of the serine residue to a proline residue). In one embodiment the Fc-region fusion polypeptide or conjugate comprises a glutamic acid residue at position 235 (mutation of the leucine residue to a glutamic acid residue).

In one embodiment the Fc-region fusion polypeptide or conjugate comprises three amino acid mutations. In one embodiment the three amino acid mutations are P329G, S228P and L235E mutation (P329G/SPLE)

In one embodiment, the further addition, mutation, or deletion of an amino acid residue in the Fc-region fusion polypeptide or conjugate as reported herein is at position 234 and/or 235 of the Fc-region if the Fc-region is of IgG1 isotype. In one embodiment the amino acid residue leucine at position 234 and/or the amino acid residue leucine at position 235 is/are mutated to another amino acid.

In one embodiment the Fc-region fusion polypeptide or conjugate comprises an Fc-region comprising an amino acid mutation at position 234, wherein the leucine amino acid residue is mutated to an alanine amino acid residue.

In one embodiment the Fc-region fusion polypeptide or conjugate comprises an Fc-region comprising an amino acid mutation at position 235, wherein the leucine amino acid residue is mutated to a serine amino acid residue.

In one embodiment the Fc-region fusion polypeptide or conjugate comprises an Fc-region comprising an amino acid mutation at position 329, wherein the proline amino acid residue is mutated to a glycine amino acid residue, an amino acid mutation at position 234, wherein the leucine amino acid residue is mutated to an alanine amino acid residue, and an amino acid mutation at position 235, wherein the leucine amino acid residue is mutated to an alanine amino acid residue.

While in one embodiment the binding to an FcγR is altered, Fc-region fusion polypeptides or conjugates with altered binding affinity for the neonatal receptor (FcRn) are also an embodiment of the aspects as reported herein.

Fc-region variants with increased affinity for FcRn have longer serum half-lives, and such molecules will have useful applications in methods of treating mammals where long half-life of the administered Fc-region fusion polypeptide or conjugate is desired, e.g., to treat a chronic disease or disorder.

Fc-region fusion polypeptides or conjugates with decreased FcRn binding affinity have shorter serum half-lives, and such molecules will have useful applications in methods of treating mammals where shorter half-life of the administered Fc-region fusion polypeptide or conjugate is desired, e.g. to avoid toxic side effects or for in vivo diagnostic imaging applications. Fc-region fusion polypeptides or conjugates with decreased FcRn binding affinity are less likely to cross the placenta, and thus may be utilized in the treatment of diseases or disorders in pregnant women.

Fc-region fusion polypeptides or conjugates with altered binding affinity for FcRn comprise in one embodiment those comprising an Fc-region with an amino acid alteration at one or more of the amino acid positions 238, 252, 253, 254, 255, 256, 265, 272, 286, 288, 303, 305, 307, 309, 311, 312, 317, 340, 356, 360, 362, 376, 378, 380, 382, 386, 388, 400, 413, 415, 424, 433, 434, 435, 436, 439, and/or 447.

Fc-region fusion polypeptides or conjugates with reduced binding to FcRn comprise in one embodiment an Fc-region with one or more amino acid alterations at the amino acid positions 252, 253, 254, 255, 288, 309, 386, 388, 400, 415, 433, 435, 436, 439, and/or 447.

Fc-region fusion polypeptides or conjugates which display increased binding to FcRn comprise in one embodiment an Fc-region with one or more amino acid alterations at the amino acid positions 238, 256, 265, 272, 286, 303, 305, 307, 311, 312, 317, 340, 356, 360, 362, 376, 378, 380, 382, 413, 424, and/or 434.

The Fc-region fusion polypeptide or conjugate may comprise an Fc-region of any class (for example, but not limited to IgG, IgM, and IgE). In one embodiment the Fc-region fusion polypeptide or conjugate comprises an Fc-region of the IgG class. In one embodiment the Fc-region fusion polypeptide or conjugate comprises an Fc-region of the IgG1, IgG2, IgG3, or IgG4 subclass.

In one embodiment the Fc-region fusion polypeptide or conjugate comprises an Fc-region of the IgG1 subclass and comprise the amino acid mutations P329G, and/or L234A and L235A in the Fc-region.

In one embodiment the Fc-region fusion polypeptide or conjugate comprises an Fc-region of the IgG4 subclass. In one embodiment the Fc-region fusion polypeptide or conjugate comprises an Fc-region of the IgG4 subclass and comprises the amino acid mutations P329G, and/or S228P and L235E in the Fc-region.

In one embodiment the Fc-region fusion polypeptide or conjugate as reported herein is produced by recombinantly fusing or conjugating a biologically active polypeptide with an Fc-region comprising one or more of the amino acid mutations as reported herein. In one embodiment the Fc-region fusion polypeptide or conjugate as reported herein is produced by modifying a parent Fc-region fusion polypeptide or conjugate by introducing one or more of the amino acid mutations as reported herein.

### Enzymatic Conjugation Using Sortase A

A conjugate comprising an Fc-region and one or more incretin receptor ligand polypeptides can be obtained by using the enzyme Sortase A.

Sortase A (SrtA) is a membrane bound enzyme which attaches proteins covalently to the bacterial cell wall. The specific recognition motif on the SrtA substrate is LPXTG (SEQ ID NO: 75), whereby the enzyme cleaves between the residues threonine and glycine. The recognition motif on the peptidoglycan is a pentaglycine motif. It has been shown that a triglycine and even a diglycine motif on the N-terminus is sufficient to support the SrtA reaction (Clancy, K. W., et al., Peptide science 94 (2010) 385-396). The reaction proceeds through a thioester acyl-enzyme intermediate, which is resolved by the attack of an amine nucleophile from the oligoglycine, covalently linking peptidoglycan to a protein substrate and regenerating SrtA. SrtA can be used to covalently conjugate chemically synthetized peptides to recombinantly expressed proteins.

For the enzymatic conjugation of an incretin receptor ligand polypeptide (e.g. with GIP receptor and GLP-1 receptor dual agonistic activity) to a human Fc-region of the subclass IgG1 a soluble SrtA (amino acid residues 60-206 of Staph. aureus SrtA) can be used. The enzyme can be produced in E. coli. The Fc-region with an N-terminal triple G motif at each heavy chain can be expressed in eukaryotic cells (e.g. HEK293 cells, CHO cells). The SrtA recognition motif is introduced at the C-terminus of the incretin receptor ligand polypeptide.

One aspect as reported herein is an Fc-region incretin receptor ligand polypeptide conjugate that is obtained by conjugating the incretin receptor ligand polypeptides to the Fc-region using the enzyme Sortase A, wherein a sortase recognition sequence is located at the C-terminus of the incretin receptor ligand polypeptide and/or the C-terminus of one or both Fc-region heavy chain fragments, and wherein a triple glycine motif is located either at the N-terminus of the incretin receptor ligand polypeptide and/or at the N-terminus of one or both Fc-region heavy chain fragments.

Accordingly, the invention provides a polypeptide comprising the amino acid sequence of the incretin receptor ligand polypeptide and the amino acid sequence of a sortase recognition sequence. In exemplary aspects, the invention provides a polypeptide comprising the amino acid sequence of an incretin receptor ligand polypeptide and LPXTG (SEQ ID NO: 75), wherein X is any amino acid. In exemplary aspects, the X is an acidic amino acid, e.g., Asp, Glu. In exemplary aspects, the X is Glu. In exemplary aspects, the polypeptide comprises one or more Gly residues N-terminally to LPXTG (SEQ ID NO: 75), wherein X is any amino acid. In alternative or additional embodiments, the polypeptide comprises Gly-Gly or Gly-Gly-Ser or Gly-Gly-Gly, Gly-Gly-Gly-Ser (SEQ ID NO: 79), Gly-Gly-Gly-Gly (SEQ ID NO: 80), or Gly-Gly-Gly-Gly-Ser (SEQ ID NO: 81)C-terminally to LPXTG (SEQ ID NO: 73). In exemplary aspects, the polypeptide comprises (GGS)ₙ, wherein n=1-4 (SEQ ID NOs: 82-84), or Gₙ, wherein n=2-6 (SEQ ID NOs: 85-87), (GGGS)ₙ, wherein n=1-6 (SEQ ID NOs: 57-60, 88 and 89), (GGGGS)ₘ, wherein m=1-6 (SEQ ID NOs: 61-64, 90 and 91, or (GGGGGS)ₒ, wherein o=1-6 (SEQ ID NOs: 65-67 and 92-94).

In one embodiment one or both of the Fc-region heavy chain fragments comprises a linker polypeptide located between the C-terminus of the triple G motif and the N-terminus of the Fc-region heavy chains.

In one embodiment the incretin receptor ligand polypeptide comprises a linker polypeptide located between the N-terminus of the SrtA recognition sequence and the C-terminus of the incretin receptor ligand polypeptide.

In one embodiment the linker polypeptide has a length of from 9 to 25 amino acid residues. In one embodiment the linker polypeptide is selected from (GGGS)₃ (SEQ ID NO: 57), (GGGS)₄ (SEQ ID NO: 58), (GGGS)₅ (SEQ ID NO: 59), (GGGS)₆ (SEQ ID NO: 60), (GGGGS)₂ (SEQ ID NO: 61), (GGGGS)₃ (SEQ ID NO: 62), (GGGGS)₄ (SEQ ID NO: 63), (GGGGS)₅ (SEQ ID NO: 64), (GGGGGS)₂ (SEQ ID NO: 65), (GGGGGS)₃ (SEQ ID NO: 66), and (GGGGGS)₄ (SEQ ID NO: 67).

In exemplary aspects, the invention provides a polypeptide comprising the amino acid sequence of the incretin receptor ligand polypeptide and the linker, e.g. a linker comprising the amino acid sequence of any of SEQ ID NOs: 57-67.

In one embodiment the fusion polypeptide or conjugate comprises one incretin receptor ligand polypeptide. In this embodiment the incretin receptor ligand polypeptide is conjugated to a single N- or C-terminus of the Fc-region. Also in this embodiment the Fc-region is a heterodimer of two antibody heavy chain Fc-region fragments whereof only one comprises the incretin receptor ligand polypeptide or an oligoglycine motif.

Conjugates comprising a human IgG1 Fc-region conjugated to two incretin receptor ligand polypeptides which have dual agonistic properties by activating the GIP receptor and the GLP-1 receptor can be used to control blood glucose level and for robust fat mass loss.

It has been shown that such a conjugate has in diabetic db/db mice resulted in reducing the blood glucose excursion following an intraperitoneal glucose challenge. In addition, in diet-induced obese (DIO) mice, it has been observed that administration of such an incretin receptor ligand polypeptide Fc-region conjugate is able to induce reduced food uptake and robust body weight loss following a single dose.

The activation of incretin receptors, such as the GLP-1- and GIP-receptors, results in glucose-dependent insulin secretion, proliferation, and protection of pancreatic beta cells from lipotoxicity and prevention of apoptosis that is mediated by pathways downstream of PKA and/or EPAC activation (Dzhura, I., et al., Islets 3 (2011) 121-128; Ehses, J. A., et al., Endocrin. 144 (2003) 4433-4445; Kang, G., et al., J. Biol. Chem. 278 (2003) 8279-8285; Miura, Y. and Matsui, H., Tox. Appl. Pharmacol. 216 (2006) 363-372; Mukai, E., et al., Diabetes 60 (2011) 218-226; Natalicchio, A., et al., Endocrin. 151 (2010) 2019-2029; Quoyer, J., et al., J. Biol. Chem. 285 (2010) 1989-2002; Uhles, S., et al., Diabetes Obes. Metabol. 13 (2010) 326-336).

Further, incretin receptors such as the GLP-1 and GIP receptors have been detected in the pancreatic alpha-cells that secrete glucagon.

The presence of incretin receptors, such as the GLP-1 receptor, has been reported in the vagus nerve as well as a wide distribution in the CNS. Activation of the portal GLP-1 receptors is reported to play a critical role in glucose homeostasis (Burcelin, R., et al., Diabetes 50 (2001) 1720-1728; Vahl, T. P., et al., Endocrin. 148 (2007) 4965-4973). In addition, GLP-1 receptors expressed in the arcuate nucleus have been implicated in regulating glucose levels (Sandoval, D. A., et al., Diabetes 57 (2008) 2046-2054).

Activation of GLP-1 receptors in the hind brain and in the hypothalamus plays an important role in limiting food consumption and prevention of obesity (Hayes, M. R., et al., Endocrinol. 150 (2009) 2654-2659; McMahon, L. R. and Wellman, P. J., Am. J. Physiol. 274 (1998) R23-29; Turton, M. D., et al., Nature 379 (1996) 69-72).

GIP and GIP-receptors are present in the CNS. GIP in the CNS is thought to play a role in neurogenesis and memory (Figueiredo, C. P., et al., Behav. Pharmacol. 21 (2010) 394-408; Nyberg, J., et al., J. Neurosci. 25 (2005) 1816-1825).

Incretin receptors, such as the GIP receptor, are present on adipocytes and induce lipolysis and re-esterification of fatty acids (Getty-Kushik, L., et al., Obesity 14 (2006) 1124-1131). In addition, GIP receptor activation leads to increased LPL expression on human adipocytes (Kim, S. J., et al., J. Biol. Chem. 282 (2007) 8557-8567; Kim, S. J., et al., J. Lipid Res. 51 (2010) 3145-3157).

### Reduced Binding to Fc Ligands

One skilled in the art will understand that the Fc-region fusion polypeptide or conjugate as reported herein has altered (relative to an unmodified Fc-region fusion polypeptide or conjugate) FcγR and/or C1q binding properties (examples of binding properties include but are not limited to, binding specificity, equilibrium dissociation constant (K_{D}), dissociation and association rates (k_{off} and kₒₙ, respectively) binding affinity and/or avidity) and that certain alterations are more or less desirable. It is known in the art that the equilibrium dissociation constant (KD) is defined as kd/ka. One skilled in the art can determine which kinetic parameter is most important for a given application. For example, a modification that reduces binding to one or more positive regulators (e.g., FcγRIIIA) and/or enhanced binding to an inhibitory Fc receptor (e.g., FcγRIIB) would be suitable for reducing ADCC activity. Accordingly, the ratio of binding affinities (e.g., equilibrium dissociation constants (KD)) can indicate if the ADCC activity is enhanced or decreased. Additionally, a modification that reduces binding to C1q would be suitable for reducing or eliminating CDC activity.

The affinities and binding properties of an Fc-region for its ligand, may be determined by a variety of in vitro assay methods (biochemical or immunological based assays) known in the art for determining Fc-region/FcR interactions, i.e., specific binding of an Fc-region to an FcγR including but not limited to, equilibrium methods (e.g. enzyme-linked immuno absorbent assay (ELISA) or radioimmunoassay (RIA)), or kinetics (e.g. BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., (ed.), Fundamental Immunology, 4^{th} Ed., Lippincott-Raven, Philadelphia (1999).

In one embodiment the Fc-region fusion polypeptide or conjugate as reported herein comprising a variant Fc-region, in which the amino acid residue proline at amino acid position 329 is mutated and in which at least one further amino acid residue is mutated, exhibits a reduced affinity to a human Fc receptor (FcR) and/or human complement compared to the Fc-region fusion polypeptide or conjugate comprising the parent Fc-region. In one embodiment the Fc-region fusion polypeptide or conjugate as reported herein has an affinity for an Fc receptor that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than for an Fc-region fusion polypeptide or conjugate comprising a wild-type human Fc-region.

In one embodiment the Fc-region fusion polypeptide or conjugate has a reduced binding affinity for one or more Fc receptors including, but not limited to FcγRI (CD64) including isoforms FcγRIA, FcγRII and FcγRIII (CD 16, including isoforms FcγRIIIA) compared to an Fc-region fusion polypeptide or conjugate comprising a wild-type Fc-region.

In one embodiment the Fc-region fusion polypeptide or conjugate has a reduced binding affinity for FcγRI (CD64) FcγRIIA and FcγRIIIA compared to the Fc-region fusion polypeptide or conjugate comprising a wild-type Fc-region.

In one embodiment the Fc-region fusion polypeptide or conjugate has a reduced binding affinity for FcγRIIA and FcγRIIIA compared to the Fc-region fusion polypeptide or conjugate comprising a wild-type Fc-region.

In one embodiment the Fc-region fusion polypeptide or conjugate has a reduced binding affinity for FcγRI (CD64) and FcγRIIIA compared to the Fc-region fusion polypeptide or conjugate comprising a wild-type Fc-region.

In one embodiment the Fc-region fusion polypeptide or conjugate has a reduced binding affinity for at least one of the Fc receptors and a reduced affinity to the C1q compared to the Fc-region fusion polypeptide or conjugate comprising a wild-type Fc-region.

In one embodiment the Fc-region fusion polypeptide or conjugate does not have an increased binding to the FcγRIIB receptor compared to the Fc-region fusion polypeptide or conjugate comprising a wild-type Fc-region.

In one embodiment the Fc-region fusion polypeptide or conjugate has an increased affinity to the human receptor FcγRIIIA, and to at least one further receptor of the group comprising the human receptors FcγIIA, FcγRIIIB, and C1q compared to the Fc-region fusion polypeptide or conjugate comprising a wild-type Fc-region.

In one embodiment the Fc-region fusion polypeptide or conjugate has a reduced affinity to the human receptor FcγRIIIA, and to at least two further receptors of the group comprising the human receptors FcγIIA, FcγRIIIB, and C1q compared to the Fc-region fusion polypeptide or conjugate comprising a wild-type Fc-region.

In one embodiment the Fc-region fusion polypeptide or conjugate has a reduced affinity to the human FcγRIA, FcγRIIIA, FcγIIA, FcγRIIIB, and C1q compared to the Fc-region fusion polypeptide or conjugate comprising a wild-type Fc-region.

In one embodiment the Fc-region fusion polypeptide or conjugate has a reduced affinity to the human receptor FcγRIA, FcγRIIIA, FcγIIA, FcγRIIIB, and C1q compared to the Fc-region fusion polypeptide or conjugate comprising a wild-type Fc-region.

In one embodiment the Fc-region fusion polypeptide or conjugate has a decreased affinity to FcγRI or FcγRIIA compared to the Fc-region fusion polypeptide or conjugate comprising a wild-type Fc-region. In one embodiment the Fc-region fusion polypeptide or conjugate has affinities for FcγRI or FcγRIIA that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of the Fc-region fusion polypeptide or conjugate comprising a wild-type Fc-region.

In one embodiment the Fc-region fusion polypeptide or conjugate has an affinity for the FcγRI or FcγRIIA that is at least 90%, at least 80%, at least 70%, at least 60%, at least 50%, at least 40%, at least 30%, at least 20%, at least 10%, or at least 5% less than that of the Fc-region fusion polypeptide or conjugate comprising a wild-type Fc-region.

In one embodiment the Fc-region fusion polypeptide or conjugate has a decreased affinity for the FcγRIIIA compared to the Fc-region fusion polypeptide or conjugate comprising a wild-type Fc-region. In one embodiment the affinity for FcγRIIIA is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of the Fc-region fusion polypeptide or conjugate comprising a wild-type Fc-region.

In one embodiment the Fc-region fusion polypeptide or conjugate has an affinity for FcγRIIIA that is at least 90%, at least 80%, at least 70%, at least 60%, at least 50%, at least 40%, at least 30%, at least 20%, at least 10%, or at least 5% less than that of the Fc-region fusion polypeptide or conjugate comprising a wild-type Fc-region.

It is understood in the art that the F1-58V allelic variant of the FcγRIIIA has altered binding characteristics to Fc-regions. In one embodiment the Fc-region fusion polypeptide or conjugate has a decreased affinity to FcγRIIIA (F1-58V) receptors compared to the Fc-region fusion polypeptide or conjugate comprising a wild-type Fc-region. In one embodiment the affinity for FcγRIIIA (F1 58V) is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of the Fc-region fusion polypeptide or conjugate comprising a wild-type Fc-region.

In one embodiment the Fc-region fusion polypeptide or conjugate has a decreased affinity for C1q compared to the Fc-region fusion polypeptide or conjugate comprising a wild-type Fc-region. In one aspect the affinity for C1q is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of the Fc-region fusion polypeptide or conjugate comprising a wild-type Fc-region.

In one embodiment the Fc-region fusion polypeptide or conjugate has an affinity for C1q that is at least 90%, at least 80%, at least 70%, at least 60%, at least 50%, at least 40%, at least 30%, at least 20%, at least 10%, or at least 5% less than that of the Fc-region fusion polypeptide or conjugate comprising a wild-type Fc-region.

In one embodiment the Fc-region fusion polypeptide or conjugate has affinities for the human FcγRI, FcγRIIA, FcγRIIIA, FcγRIIIA (F1 58V) or C1q that are at least 90%, at least 80%, at least 70%, at least 60%, at least 50%, at least 40%, at least 30%, at least 20%, at least 10%, or at least 5% less than that of the Fc-region fusion polypeptide or conjugate comprising a wild-type Fc-region.

In one embodiment the Fc-region fusion polypeptide or conjugate has affinities for the FcγRI, FcγRIIA, FcγRIIIA, FcγRIIIA (F1-58V), and/or C1q, respectively, that are between about 10 nM to 100 nM, 10 nM to 1 μM, 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In one embodiments the affinities for the FcγRI, FcγRIIA, FcγRIIIA, FcγRIIIA (F1-58V), or C1q are greater than 100 nM, 500 nM, 1 μM, greater than 5 μM, greater than 10 μM, greater than 25 μM, greater than 50 μM, or greater than 100 μM.

In one embodiment the Fc-region fusion polypeptide or conjugate has increased affinity for the FcγRIIB the Fc-region fusion polypeptide or conjugate comprising a wild-type Fc-region. In one embodiment the affinity for the FcγRIIB is unchanged or increased by at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold than that of the Fc-region fusion polypeptide or conjugate comprising a wild-type Fc-region. In one embodiment the affinity for the FcγRIIB receptor is increased by at least 5%, at least 10%, at least 20%, at least 30%, at least 40%, at least 50%, at least 60%, at least 70%, at least 80%, at least 90%, or at least 95% compared to the Fc-region fusion polypeptide or conjugate comprising a wild-type Fc-region.

In one embodiment the Fc-region fusion polypeptide or conjugate has affinities for the FcγRI, FcγRIIA FcγRIIIA, or FcγRIIIA (F1-58V), or C1q that are less than 100 μM, less than 50 μM, less than 10 μM, less than 5 μM, less than 2.5 μM, less than 1 μM, or less than 100 nM, or less than 10 nM.

### Reduced Effector Function

In a certain aspect of the invention the fusion polypeptide or conjugate as reported herein modulates an effector function as compared to the fusion polypeptide or conjugate comprising the wild-type Fc-region.

In one embodiment the modulation is a modulation of ADCC, and/or ADCP, and/or CDC.

In one embodiment the modulation is down-modulation or reduction in effect.

In one embodiment the modulation is a modulation of ADCC. In one embodiment the modulation is a down-modulation of ADCC and/or ADCP.

In one embodiment the modulation is a down-modulation of ADCC and CDC. In one embodiment the modulation is a down-modulation of ADCC only. In one embodiment the modulation is a down-modulation of ADCC and CDC, and/or ADCP. In one embodiment the modulation is a down-modulation or reduction of ADCC, CDC, and ADCP.

In one embodiment the reduction or down-modulation of ADCC, and/or CDC, and/or ADCP is a reduction to 0%, 2.5%, 5%, 10%, 20%, 50%, or 75% of the value observed for induction of ADCC, and/or CDC, and/or ADCP, respectively, by the fusion polypeptide or conjugate comprising the wild-type Fc-region.

In one embodiment the modulation of ADCC is a decrease in potency such that the EC₅₀ value of the fusion polypeptide or conjugate is at least about 10-fold reduced compared to the fusion polypeptide or conjugate comprising the wild-type Fc-region.

In one embodiment the fusion polypeptide or conjugate as reported herein is substantially devoid of ADCC, and/or CDC, and/or ADCP in the presence of human effector cells compared to the fusion polypeptide or conjugate comprising a wild-type Fc-region.

In one embodiment the fusion polypeptide or conjugate as reported herein has a reduced, for example reduction by at least 20%, or strongly reduced, for example reduction by at least 50%, effector function, which could be a down-modulation or reduction in ADCC, CDC, and/or ADCP compared to the fusion polypeptide or conjugate comprising a wild-type Fc-region.

### Reduced ADCC Activity

In vitro and/or in vivo cytotoxicity assays can be used to determine the reduction/depletion of CDC and/or ADCC activities. For example, Fc receptor (FcR) binding assays can be used to ensure that the fusion polypeptide or conjugate lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9 (1991) 457-492. Non-limiting examples of in vitro assays to assess ADCC activity of a molecule of interest are described e.g. in U.S. Pat. No. 5,500,362, Hellstrom, I., et al., Proc. Natl. Acad. Sci. USA 83 (1986) 7059-7063, Hellstrom, I., et al., Proc. Natl. Acad. Sci. USA 82 (1985) 1499-1502, U.S. Pat. No. 5,821,337, or Bruggemann, M., et al., J. Exp. Med. 166 (1987) 1351-1361. Non-radioactive assays methods may also be employed. For example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.) can be used. Useful effector cells for such assays include peripheral blood mononuclear cells (PBMC) and Natural Killer (NK) cells. Alternatively, or additionally, ADCC activity of the fusion polypeptide or conjugate can be assessed in vivo, e.g., in an animal model such as that reported in Clynes, et al., Proc. Natl. Acad. Sci. USA 95 (1998) 652-656. C1q binding assays may also be carried out to confirm that the fusion polypeptide or conjugate does not bind C1q and hence lacks CDC activity. See, e.g., C1q and C3c binding ELISA reported in WO 2006/029879 and WO 2005/100402. To assess complement activation, a CDC assay may be performed (see, for example, Gazzano-Santoro, et al., J. Immunol. Meth. 202 (1996) 163; Cragg, M. S., et al., Blood 101 (2003) 1045-1052; and Cragg, M. S., and Glennie, M. J., Blood 103 (2004) 2738-2743). FcRn binding and in vivo clearance/half-life determinations can also be performed using methods known in the art (see, e.g., Petkova, S. B., et al., Int. Immunol. 18 (2006) 1759-1769).

It is contemplated that fusion polypeptides or conjugates as reported herein are characterized by in vitro functional assays for determining one or more FcγR mediated effector cell functions.

In certain embodiments, the fusion polypeptide or conjugate as reported herein has similar binding properties and effector cell functions in in vivo models (such as those described and disclosed herein) as those in in vitro based assays. However, it is not excluded that fusion polypeptide or conjugates as reported herein do not exhibit the desired phenotype in in vitro based assays but do exhibit the desired phenotype in vivo.

In one embodiment, the fusion polypeptide or conjugate as reported herein has decreased ADCC activity compared to a fusion polypeptide or conjugate comprising a wild-type Fc-region.

In one embodiment the fusion polypeptide or conjugate as reported herein has an ADCC activity that is at least 2 fold, or at least 3 fold, or at least 5 fold, or at least 10 fold, or at least 50 fold, or at least 100 fold less than that of a fusion polypeptide or conjugate comprising a wild-type Fc-region.

In one embodiment, the fusion polypeptide or conjugate as reported herein has an ADCC activity that is reduced by at least 10%, or by at least 20%, or by at least 30%, or by at least 40%, or by at least 50%, or by at least 60%, or by at least 70%, or by at least 80%, or by at least 90%, or by about 100% relative to a fusion polypeptide or conjugate comprising a wild-type Fc-region.

In one embodiment the fusion polypeptide or conjugate as reported herein has a reduced or down-modulated ADCC activity that is 0%, 2.5%, 5%, 10%, 20%, 50%, or 75% of the value observed for induction of ADCC, or CDC or ADCP, respectively, by the fusion polypeptide or conjugate comprising a wild-type Fc-region.

In one embodiment, the fusion polypeptide or conjugate as reported herein has no detectable ADCC activity.

In one embodiment, the reduction and/or ablation of ADCC activity is due to a reduced affinity of the fusion polypeptide or conjugate as reported herein to Fc ligands and/or receptors.

In one embodiment the down-modulation of ADCC is a decrease in potency such that the EC₅₀ value of the fusion polypeptide or conjugate as reported herein is approximately 10-fold reduced compared to the fusion polypeptide or conjugate comprising a wild-type Fc-region.

In one embodiment the fusion polypeptide or conjugate as reported herein modulates ADCC, and/or CDC, and/or ADCP. In one embodiment the fusion polypeptide or conjugate has a reduced CDC and ADCC, and/or ADCP activity.

### Reduced CDC Activity

The complement activation pathway is initiated by the binding of the first component of the complement system (C1q) to a molecule, an Fc-region comprising molecule for example, complexed with a cognate antigen. To assess complement activation, a CDC assay, e.g. as described in Gazzano-Santoro, et al., J. Immunol. Methods, 202 (1996) 163, may be performed.

The binding properties of different fusion polypeptides or conjugates as reported herein to C1q can be analyzed by an ELISA sandwich type immunoassay. The fusion polypeptide or conjugate concentration at the half maximum response determines the EC₅₀ value. This read-out is reported as relative difference to the reference standard measured on the same plate together with the coefficient of variation of sample and reference.

In one embodiment, the fusion polypeptide or conjugate as reported herein has a decreased affinity to C1q relative to a fusion polypeptide or conjugate comprising a wild-type Fc-region. In one embodiment, the fusion polypeptide or conjugate has an affinity for C1q that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than the affinity of a fusion polypeptide or conjugate comprising a wild-type Fc-region.

In one embodiment, the fusion polypeptide or conjugate as reported herein has an affinity for C1q that is at least 90%, or at least 80%, or at least 70%, or at least 60%, or at least 50%, or at least 40%, or at least 30%, or at least 20%, or at least 10%, or at least 5% less than that of a fusion polypeptide or conjugate comprising a wild-type Fc-region.

In one embodiment, the fusion polypeptide or conjugate as reported herein has an affinity for C1q that is between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In one embodiment the fusion polypeptide or conjugate has an affinity for C1q that is 1 μM or more, or 5 μM or more, or 10 μM or more, or 25 μM or more, or 50 μM or more, or 100 μM or more.

In one embodiment the fusion polypeptide or conjugate as reported herein has reduced CDC activity compared to a fusion polypeptide or conjugate comprising a wild-type Fc-region.

In one embodiment, the fusion polypeptide or conjugate reported herein has a CDC activity that is at least 2 fold, or at least 3 fold, or at least 5 fold, or at least 10 fold, or at least 50 fold, or at least 100 fold less than that of a fusion polypeptide or conjugate comprising a wild-type Fc-region.

In one embodiment the fusion polypeptide or conjugate as reported herein has a CDC activity that is reduced by at least 10%, or by at least 20%, or by at least 30%, or by at least 40%, or by at least 50%, or by at least 60%, or by at least 70%, or by at least 80%, or by at least 90%, or by about 100% relative to a fusion polypeptide or conjugate comprising a wild-type Fc-region.

In one embodiment the fusion polypeptide or conjugate reported herein has no detectable CDC activity.

In one embodiment, the reduction and/or ablation of CDC activity is attributed to the reduced affinity of the fusion polypeptide or conjugate for Fc ligands and/or receptors.

### Reduced Antibody Related Toxicity

It is understood in the art that biological therapies may have adverse toxicity issues associated with the complex nature of directing the immune system to recognize and attack unwanted cells and/or targets. When the recognition and/or the targeting for attack do not take place where the treatment is required, consequences such as adverse toxicity may occur. For example, antibody staining of non-targeted tissues may be indicative of potential toxicity issues.

In one embodiment, the fusion polypeptide or conjugate as reported herein has reduced antibody related toxicity as compared to a fusion polypeptide or conjugate comprising a wild-type Fc-region. In one embodiment, the fusion polypeptide or conjugate has a toxicity that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of a fusion polypeptide comprising a wild-type Fc-region. In one embodiment, the fusion polypeptide or conjugate has a toxicity that is reduced by at least 10%, or by at least 20%, or by at least 30%, or by at least 40%, or by at least 50%, or by at least 60%, or by at least 70%, or by at least 80%, or by at least 90%, or by about 100% relative to a fusion polypeptide or conjugate comprising a wild-type Fc-region.

### Thrombocyte Aggregation

In one embodiment a fusion polypeptide or conjugate as reported herein has compared to a fusion polypeptide or conjugate comprising a wild-type Fc-region reduced induction of platelet activation and/or platelet aggregation. In one embodiment the fusion polypeptide or conjugate as reported herein has a decreased or even ablated induction of thrombocyte activation and/or aggregation.

It is understood in the art that biological therapies may have as adverse effect thrombocyte aggregation. In vitro and in vivo assays could be used for measuring thrombocyte aggregation. It is assumed that the in vitro assay reflects the in vivo situation.

In one embodiment the fusion polypeptide or conjugate as reported herein has a reduced induction of thrombocyte aggregation in an in vitro assay compared to a fusion polypeptide or conjugate comprising a wild-type Fc-region.

In one embodiment the fusion polypeptide or conjugate has an induction of thrombocyte aggregation in an in vitro assay that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of a fusion polypeptide or conjugate comprising a wild-type Fc-region.

In one embodiment, the fusion polypeptide or conjugate as reported herein has an induction of thrombocyte aggregation in an in vitro assay that is reduced by at least 10%, or by at least 20%, or by at least 30%, or by at least 40%, or by at least 50%, or by at least 60%, or by at least 70%, or by at least 80%, or by at least 90%, or by about 100% relative to a fusion polypeptide or conjugate comprising a wild-type Fc-region.

In one embodiment the fusion polypeptide or conjugate as reported herein has a reduced in vivo induction of thrombocyte aggregation compared to a fusion polypeptide comprising a wild-type Fc-region. In one embodiment the fusion polypeptide or conjugate as reported herein has a reduced induction of thrombocyte aggregation in an in vivo assay that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than that of a fusion polypeptide or conjugate comprising a wild-type Fc-region.

In one embodiment, the fusion polypeptide or conjugate as reported herein has a reduced induction of thrombocyte aggregation in an in vivo assay that is reduced by at least 10%, or by at least 20%, or by at least 30%, or by at least 40%, or by at least 50%, or by at least 60%, or by at least 70%, or by at least 80%, or by at least 90%, or by about 100% relative to a fusion polypeptide comprising a wild-type Fc-region.

### III. Recombinant Methods

Fc-fusion polypeptides or parts of the Fc-region conjugates may be produced using recombinant methods and compositions, see e.g. U.S. Pat. No. 4,816,567.

In one aspect an isolated nucleic acid encoding a fusion polypeptide or part of a conjugate as reported herein is provided.

In one aspect one or more vectors (e.g., expression vectors) comprising such nucleic acid are provided.

In one aspect a host cell comprising such nucleic acid is provided. In one embodiment a host cell comprises (e.g., has been transformed with): (1) a vector comprising a nucleic acid that encodes an amino acid sequence comprising the first heavy chain Fc-region of the fusion polypeptide or the full or a part of the first heavy chain Fc-region of the conjugate and an amino acid sequence comprising the second heavy chain Fc-region of the fusion polypeptide or the full or a part of the second heavy chain Fc-region of the conjugate, or (2) a first vector comprising a nucleic acid that encodes an amino acid sequence comprising the first heavy chain Fc-region of the fusion polypeptide or the full or a part of the first heavy chain Fc-region of the conjugate and a second vector comprising a nucleic acid that encodes an amino acid sequence comprising the second heavy chain Fc-region of the fusion polypeptide or the full or a part of the second heavy chain Fc-region of the conjugate.

In one embodiment, the host cell is a eukaryotic cell, e.g. a human embryonic kidney (HEK) cell, or a Chinese Hamster Ovary (CHO) cell, or a lymphoid cell (e.g., Y0, NS0, Sp20 cell).

In one aspect a method of making a fusion polypeptide as reported herein is provided, wherein the method comprises culturing a host cell comprising a nucleic acid encoding the fusion polypeptide or conjugate as provided above, under conditions suitable for expression of the fusion polypeptide or conjugate, and optionally recovering the fusion polypeptide or conjugate from the host cell (or host cell culture medium).

In one aspect a method of making a polypeptide conjugate as reported herein is provided, wherein the method comprises culturing a host cell comprising a nucleic acid encoding a part of the polypeptide conjugate as provided above, under conditions suitable for expression of the part of the polypeptide conjugate, and optionally recovering the part of the polypeptide conjugate from the host cell (or host cell culture medium) and conjugating the recombinantly produced part of the polypeptide conjugate with the respective other part of the polypeptide conjugate chemically or enzymatically. The respective other part of the polypeptide conjugate can be produced recombinantly and modified thereafter or can be produced completely synthetically.

For recombinant production of a fusion polypeptide or a part of the polypeptide conjugate, nucleic acid encoding a fusion polypeptide or a part of the polypeptide conjugate, e.g., as described above, is isolated and inserted into one or more vectors for further cloning and/or expression in a host cell. Such nucleic acid may be readily isolated and/or produced using conventional procedures.

Suitable host cells for cloning or expression of polypeptide-encoding vectors include prokaryotic or eukaryotic cells described herein. For example, polypeptides may be produced in bacteria, in particular when glycosylation and Fc effector function are not needed (see, e.g., U.S. Pat. No. 5,648,237, U.S. Pat. No. 5,789,199, and U.S. Pat. No. 5,840,523, Charlton, Methods in Molecular Biology 248 (2003) 245-254 (B. K. C. Lo, (ed.), Humana Press, Totowa, N.J.), describing expression of antibody fragments in E. coli.). After expression, the polypeptide may be isolated from the bacterial cell paste in a soluble fraction and can be further purified.

In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern (see e.g. Gerngross, Nat. Biotech. 22 (2004) 1409-1414, and Li, et al., Nat. Biotech. 24 (2006) 210-215).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see, e.g., U.S. Pat. No. 5,959,177, U.S. Pat. No. 6,040,498, U.S. Pat. No. 6,420,548, U.S. Pat. No. 7,125,978, and U.S. Pat. No. 6,417,429 (describing PLANTIBODIES™ technology for producing antibodies in transgenic plants)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or kidney cells (BHK); mouse sertoli cells (TM4 cells as described, e.g., in Mather, Biol. Reprod. 23 (1980) 243-251); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather, et al., Annals N.Y. Acad. Sci. 383 (1982) 44-68; MRC 5 cells; and FS4 cells. Other useful mammalian host cell lines include Chinese hamster ovary (CHO) cells, including DHFR negative CHO cells (Urlaub, et al., Proc. Natl. Acad. Sci. USA 77 (1980) 4216), and myeloma cell lines such as Y0, NS0 and Sp2/0. For a review of certain mammalian host cell lines suitable for polypeptide production, see, e.g., Yazaki, and Wu, Methods in Molecular Biology 248 (2003) 255-268 (B. K. C. Lo, (ed.), Humana Press, Totowa, N.J.).

### IV. Pharmaceutical Formulations

Pharmaceutical formulations of a fusion polypeptide or conjugate as reported herein are prepared by mixing such fusion polypeptide or conjugate having the desired degree of purity with one or more optional pharmaceutically acceptable carriers (Osol, A., (ed.), Remington's Pharmaceutical Sciences, 16^{th} edition, (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as poly (vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and US 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, especially those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A., (ed.), (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### V. Therapeutic Methods and Compositions

Any of the fusion polypeptides or conjugates reported herein may be used in therapeutic methods.

In one aspect of the invention the fusion polypeptide or conjugate as reported herein is used for treating a disease. In one embodiment the disease is such, that it is favorable that the effector function of the fusion polypeptide or conjugate is strongly, at least by 50%, reduced compared to the fusion polypeptide or conjugate comprising a wild-type Fc-region.

In one aspect the fusion polypeptide or conjugate as reported herein is used in the manufacture of a medicament for the treatment of a disease, wherein it is favorable that the effector function of the fusion polypeptide or conjugate is strongly reduced compared to a fusion polypeptide or conjugate comprising a wild-type Fc-region.

In one aspect the fusion polypeptide or conjugate as reported herein is used in the manufacture of a medicament for the treatment of a disease, wherein it is favorable that the effector function of the fusion polypeptide or conjugate is reduced compared to a fusion polypeptide or conjugate comprising a wild-type Fc-region by at least 20%.

One aspect as reported herein is a method of treating an individual having a disease, wherein it is favorable that the effector function of the fusion polypeptide or conjugate as reported herein is strongly reduced compared to a fusion polypeptide or conjugate comprising a wild-type Fc-region, comprising administering to the individual an effective amount of the fusion polypeptide or conjugate as reported herein.

A strong reduction of effector function is a reduction of effector function by at least 50% of the effector function induced by the fusion polypeptide or conjugate comprising a wild-type Fc-region.

Such diseases are for example all diseases where the targeted cell should not be destroyed by for example ADCC, ADCP, or CDC.

The conditions which can be treated with the polypeptide variant are many and include metabolic disorders.

The fusion polypeptide or conjugate as reported herein is administered by any suitable means, including enteral (orally or rectally), gastrointestinal, sublingual, sublabial, parenteral, subcutaneous, intravenous, intradermal, intraperitoneal, intrapulmonary, and intranasal. In one embodiment the dosing is given by tablet, capsule, or droplet.

For the prevention or treatment of disease, the appropriate dosage of the fusion polypeptide or conjugate will depend on the type of disease to be treated, the severity and course of the disease, whether the fusion polypeptide or conjugate is administered for preventive or therapeutic purposes, previous therapy, the patient's clinical history and response to the fusion polypeptide or conjugate, and the discretion of the attending physician. The fusion polypeptide or conjugate is suitably administered to the patient at one time or over a series of treatments.

Depending on the type and severity of the disease, about 1 μg/kg to 15 mg/kg (e.g., 0.1-20 mg/kg) of fusion polypeptide or conjugate is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

Metabolic Syndrome, also known as metabolic syndrome X, insulin resistance syndrome or Reaven's syndrome, is a disorder that affects over 50 million Americans. Metabolic Syndrome is typically characterized by a clustering of at least three or more of the following risk factors: (1) abdominal obesity (excessive fat tissue in and around the abdomen), (2) atherogenic dyslipidemia (blood fat disorders including high triglycerides, low HDL cholesterol and high LDL cholesterol that enhance the accumulation of plaque in the artery walls), (3) elevated blood pressure, (4) insulin resistance or glucose intolerance, (5) prothrombotic state (e.g. high fibrinogen or plasminogen activator inhibitor-1 in blood), and (6) pro-inflammatory state (e.g. elevated C-reactive protein in blood). Other risk factors may include aging, hormonal imbalance and genetic predisposition.

Metabolic Syndrome is associated with an increased the risk of coronary heart disease and other disorders related to the accumulation of vascular plaque, such as stroke and peripheral vascular disease, referred to as atherosclerotic cardiovascular disease (ASCVD). Patients with Metabolic Syndrome may progress from an insulin resistant state in its early stages to full blown type II diabetes with further increasing risk of ASCVD. Without intending to be bound by any particular theory, the relationship between insulin resistance, Metabolic Syndrome and vascular disease may involve one or more concurrent pathogenic mechanisms including impaired insulin-stimulated vasodilation, insulin resistance-associated reduction in NO availability due to enhanced oxidative stress, and abnormalities in adipocyte-derived hormones such as adiponectin (Lteif and Mather, Can. J. Cardiol. 20 (suppl. B):66B-76B (2004)).

According to the 2001 National Cholesterol Education Program Adult Treatment Panel (ATP III), any three of the following traits in the same individual meet the criteria for Metabolic Syndrome: (a) abdominal obesity (a waist circumference over 102 cm in men and over 88 cm in women); (b) serum triglycerides (150 mg/dl or above); (c) HDL cholesterol (40 mg/dl or lower in men and 50 mg/dl or lower in women); (d) blood pressure (130/85 or more); and (e) fasting blood glucose (110 mg/dl or above). According to the World Health Organization (WHO), an individual having high insulin levels (an elevated fasting blood glucose or an elevated post meal glucose alone) with at least two of the following criteria meets the criteria for Metabolic Syndrome: (a) abdominal obesity (waist to hip ratio of greater than 0.9, a body mass index of at least 30 kg/m², or a waist measurement over 37 inches); (b) cholesterol panel showing a triglyceride level of at least 150 mg/dl or an HDL cholesterol lower than 35 mg/dl; (c) blood pressure of 140/90 or more, or on treatment for high blood pressure). (Mathur, Ruchi, “Metabolic Syndrome,” ed. Shiel, Jr., William C., MedicineNet.com, May 11, 2009).

For purposes herein, if an individual meets the criteria of either or both of the criteria set forth by the 2001 National Cholesterol Education Program Adult Treatment Panel or the WHO, that individual is considered as afflicted with Metabolic Syndrome.

Without being bound to any particular theory, the Fc region fusion polypeptides or Fc region polypeptide conjugates described herein are useful for treating Metabolic Syndrome. Accordingly, the invention provides a method of preventing or treating Metabolic Syndrome, or reducing one, two, three or more risk factors thereof, in a subject, comprising administering to the subject a Fc region fusion polypeptide or Fc region polypeptide conjugate described herein in an amount effective to prevent or treat Metabolic Syndrome, or the risk factor thereof.

One aspect as reported herein is a fusion polypeptide or conjugate as reported herein for use in a method of treating an individual having diabetes or obesity comprising administering to the individual an effective amount of the fusion polypeptide or conjugate as reported herein. In one embodiment, the method further comprises administering to the individual an effective amount of at least one additional therapeutic agent.

In one aspect a fusion polypeptide or conjugate as reported herein is provided for use in stimulation of insulin synthesis and/or secretion, inhibition of glucagon secretion, inhibition of food intake, or/and reduction of hyperglycemia in an individual comprising administering to the individual an effective dose of the fusion polypeptide or conjugate as reported herein to stimulate insulin synthesis and/or secretion, inhibit glucagon secretion, inhibit of food intake, or/and reduce hyperglycemia in an individual. In one embodiment the individual is a human.

In one aspect methods for inducing weight loss or preventing weight gain are provided herein, which involve administering to a patient in need thereof an effective amount of a fusion polypeptide or conjugate as reported herein, that exhibits activity at both the GIP receptor and the GLP-I receptor, and that optionally also exhibits activity at the glucagon receptor. Such compounds include the GIP/GLP-1 co-agonists and glucagon/GIP/GLP-1 tri-agonists described herein.

One aspect as reported herein is the use of a fusion polypeptide or conjugate as reported herein in the manufacture or preparation of a medicament. In one embodiment, the medicament is for treatment of diabetes or obesity. In a further embodiment, the medicament is for use in a method of treating diabetes or obesity comprising administering to an individual having diabetes or obesity an effective amount of the medicament. In one embodiment the method further comprises administering to the individual an effective amount of at least one additional therapeutic agent. In a further embodiment the medicament is for stimulation of insulin synthesis and/or secretion, inhibition of glucagon secretion, inhibition of food intake, or/and reduction of hyperglycemia.

In a further embodiment, the medicament is for use in a method of stimulating insulin synthesis and/or secretion, inhibiting glucagon secretion, inhibiting food intake, or/and reducing hyperglycemia in an individual comprising administering to the individual an amount effective of the medicament to stimulate insulin synthesis and/or secretion, inhibit glucagon secretion, inhibit food intake, or/and reduce hyperglycemia. An “individual” according to any of the above embodiments may be a human.

Nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). Simple fatty liver is the abnormal accumulation of a certain type of fat, triglyceride, in the liver cells with no inflammation or scarring. In NASH, the fat accumulation is associated with varying degrees of inflammation (hepatitis) and scarring (fibrosis) of the liver. The inflammatory cells can destroy the liver cells (hepatocellular necrosis). In the terms “steatohepatitis” and “steatonecrosis”, steato refers to fatty infiltration, hepatitis refers to inflammation in the liver, and necrosis refers to destroyed liver cells. NASH can ultimately lead to scarring of the liver (fibrosis) and then irreversible, advanced scarring (cirrhosis). Cirrhosis that is caused by NASH is the last and most severe stage in the NAFLD spectrum. (Mendler, Michel, “Fatty Liver: Nonalcoholic Fatty Liver Disease (NAFLD) and Nonalcoholic Steatohepatitis (NASH),” ed. Schoenfield, Leslie J., MedicineNet.com, Aug. 29, 2005).

Alcoholic Liver Disease, or Alcohol-Induced Liver Disease, encompasses three pathologically distinct liver diseases related to or caused by the excessive consumption of alcohol: fatty liver (steatosis), chronic or acute hepatitis, and cirrhosis. Alcoholic hepatitis can range from a mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma. Histologically, alcoholic hepatitis has a characteristic appearance with ballooning degeneration of hepatocytes, inflammation with neutrophils and sometimes Mallory bodies (abnormal aggregations of cellular intermediate filament proteins). Cirrhosis is characterized anatomically by widespread nodules in the liver combined with fibrosis. (Worman, Howard J., “Alcoholic Liver Disease”, Columbia University Medical Center website).

Without being bound to any particular theory, the Fc region fusion polypeptides or Fc region polypeptide conjugates described herein are useful for the treatment of Alcoholic Liver Disease, NAFLD, or any stage thereof, including, for example, steatosis, steatohepatitis, hepatitis, hepatic inflammation, NASH, cirrhosis, or complications thereof. Accordingly, the invention provides a method of preventing or treating Alcoholic Liver Disease, NAFLD, or any stage thereof, in a subject comprising administering to a subject a Fc region fusion polypeptide or Fc region polypeptide conjugate described herein in an amount effective to prevent or treat Alcoholic Liver Disease, NAFLD, or the stage thereof. Such treatment methods include reduction in one, two, three or more of the following: liver fat content, incidence or progression of cirrhosis, incidence of hepatocellular carcinoma, signs of inflammation, e.g. abnormal hepatic enzyme levels (e.g., aspartate aminotransferase AST and/or alanine aminotransferase ALT, or LDH), elevated serum ferritin, elevated serum bilirubin, and/or signs of fibrosis, e.g. elevated TGF-beta levels. In preferred embodiments, the Fc region fusion polypeptides or Fc region polypeptide conjugates are used treat patients who have progressed beyond simple fatty liver (steatosis) and exhibit signs of inflammation or hepatitis. Such methods may result, for example, in reduction of AST and/or ALT levels.

In one aspect herein is provided a pharmaceutical formulation comprising any of the fusion polypeptides or conjugates as reported herein, e.g., for use in any of the above therapeutic methods. In one embodiment, a pharmaceutical formulation comprises any of the fusion polypeptides or conjugates provided herein and a pharmaceutically acceptable carrier. In one embodiment a pharmaceutical formulation comprises any of the fusion polypeptides or conjugates provided herein and at least one additional therapeutic agent.

Fusion polypeptides or conjugates as reported herein can be used either alone or in combination with other agents in a therapy. For instance, a fusion polypeptide or conjugate as reported herein may be co-administered with at least one additional therapeutic agent.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

Fusion polypeptides or conjugates as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion polypeptide or conjugate need not be, but is optionally formulated with, one or more agents currently used to prevent or treat the disorder in question. The effective amount of such other agents depends on the amount of the fusion polypeptide or conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a fusion polypeptide or conjugate as reported herein (when used alone or in combination with one or more other additional therapeutic agents) will depend on the type of disease to be treated, the type of fusion polypeptide or conjugate, the severity and course of the disease, whether the fusion polypeptide or conjugate is administered for preventive or therapeutic purposes, previous therapy, the patient's clinical history and response to the fusion polypeptide or conjugate, and the discretion of the attending physician. The fusion polypeptide or conjugate is suitably administered to the patient at one time or over a series of treatments. One exemplary dosage of the fusion polypeptide or conjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion polypeptide or conjugate). An initial higher loading dose, followed by one or more lower doses may be administered. However, other dosage regimens may be useful. The progress of this therapy is easily monitored by conventional techniques and assays.

### VI. Articles of Manufacture

In another aspect of the invention, an article of manufacture containing materials useful for the treatment, and/or prevention of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, and/or preventing the condition and may have a sterile access port (for example the container may be an intravenous solution bag or a vial having a stopper pierceable by a hypodermic injection needle). At least one active agent in the composition is a fusion polypeptide or conjugate as reported herein. The label or package insert indicates that the composition is used for treating the condition of choice. Moreover, the article of manufacture may comprise (a) a first container with a composition contained therein, wherein the composition comprises a fusion polypeptide or conjugate as reported herein; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The disclosures of all patent and scientific literature cited herein are expressly incorporated in their entirety by reference.

### Description of the Sequence Listing:

SEQ ID NO: 01 to 39 incretin receptor ligand polypeptide

SEQ ID NO: 40 human immunoglobulin heavy chain CH2 domain

SEQ ID NO: 42 human immunoglobulin heavy chain CH3 domain

SEQ ID NO: 43 human Fc-region of IgG1 isotype

SEQ ID NO: 44 to 53 variant human Fc-regions of IgG1 isotype

SEQ ID NO: 54 human Fc-region of IgG4 isotype

SEQ ID NO: 55 and 56 variant human Fc-regions of IgG4 isotype

SEQ ID NO: 57 to 67 linker polypeptides

SEQ ID NO: 68 exemplary incretin receptor ligand polypeptide Fc-region conjugate without a linker

SEQ ID NO: 69 exemplary incretin receptor ligand polypeptide Fc-region conjugate comprising a linker

SEQ ID NO: 70 long incretin receptor ligand polypeptide with sortase tag

SEQ ID NO: 71 short incretin receptor ligand polypeptide with sortase tag

SEQ ID NO: 72 sortase tag

### EXAMPLES

The following examples are examples of methods and compositions of the invention. It is understood that various other embodiments may be practiced, given the general description provided above.

Although the foregoing invention has been described in some detail by way of illustration and example for purposes of clarity of understanding, the descriptions and examples should not be construed as limiting the scope of the invention.

### Example 1

### Antibodies

For the experiments described below antibodies against CD9 (see SEQ IDs 8-14 in PCT/EP2012/055393), P-selectin (sequences described in WO 2005/100402) and CD20 (sequences described in EP 1 692 182) were used.

All variants described herein, e.g. P329G, P329A, P329R SPLE, LALA, P329G/LALA, P329G/SPLE variants of the anti-P-selectin antibody, anti-CD9 antibody, and anti-CD20 antibody (numbering according to EU index of Kabat) were prepared using PCR based mutagenesis. IgG molecules were expressed in HEK-EBNA or HEK293 (anti-CD9 antibody) cells, and purified using protein A and size exclusion chromatography.

### Example 2

### Determination of the Binding Affinities of Different Fcγ Receptors to Immunoglobulins

Binding affinities of different FcγRs towards immunoglobulins were measured by Surface Plasmon Resonance (SPR) using a BIAcore T100 instrument (GE Healthcare) at 25° C.

The BIAcore® system is well established for the study of molecule interactions. It allows a continuous real-time monitoring of ligand/analyte bindings and thus the determination of association rate constants (kₐ), dissociation rate constants (k_{d}), and equilibrium constants (K_{D}). Changes in the refractive index indicate mass changes on the surface caused by the interaction of immobilized ligand with analyte injected in solution. If molecules bind immobilized ligands on the surface the mass increases, in case of dissociation the mass decreases.

For a 1:1 interaction no difference in the results should be seen if a binding molecule is either injected over the surface or immobilized onto a surface. Therefore different settings were used (with Fcγ receptor as ligand or analyte respectively), depending on solubility and availability of ligand or corresponding analyte.

For FcγRI 10,000 resonance units (RU) of a capturing system recognizing a poly-histidine sequence (pentaHis monoclonal antibody, Qiagen Hilden, cat. no. 34660) was immobilized by the use of an amine coupling kit supplied by the GE Healthcare and a CM5 chip at pH 4.5. FcγRI was captured at a concentration of 5 μg/ml by with a pulse of 60 sec at a flow of 5 μl/min. Different concentrations of antibodies ranging from 0 to 100 nM were passed with a flow rate of 30 μl/min through the flow cells at 298 K for 120 sec to record the association phase. The dissociation phase was monitored for up to 240 sec and triggered by switching from the sample solution to running buffer. The surface was regenerated by 2 mM washing with a glycine solution at pH 2 at a flow rate of 30 ml/min. For all experiments HBS-P+ buffer supplied by GE Healthcare was chosen (10 mM HEPES, pH 7.4, 150 mM NaCl, 0.05% (v/v) Surfactant P20). Bulk refractive index differences were corrected for by subtracting the response obtained from a surface without captured FcγRI. Blank injections are also subtracted (=double referencing).

The equilibrium dissociation constant (K_{D}), defined as kₐ/k_{d}, was determined by analyzing the sensorgram curves obtained with several different concentrations, using BIAevaluation software package. The fitting of the data followed a suitable binding model.

For FcγRIIA and FcγRIIIAV158 10,000 resonance units (RU) of a monoclonal antibody to be tested was immobilized onto a CMS chip by the use of an amine coupling kit supplied by the GE (pH 4.5 at a concentration of 10 μg/ml).

Different concentrations of FcγRIIA and MA ranging from 0 to 12.8 μM were passed with a flow rate of 5 μl/min through the flow cells at 298 K for 120 sec to record the association phase. The dissociation phase was monitored for up to 240 sec. and triggered by switching from the sample solution to running buffer. The surface was regenerated by 0.5 min washing with a 3 mM NaOH/1M NaCl solution at a flow rate of 30 ml/min. For all experiments HBS-P+ buffer supplied by GE Healthcare was chosen (10 mM HEPES, pH 7.4, 150 mM NaCl, 0.05% (v/v) Surfactant P20).

Bulk refractive index differences were corrected for by subtracting the response obtained from a surface without captured antibody. Blank injections are also subtracted (=double referencing).

The equilibrium dissociation constant (K_{D}), was determined by analyzing the sensorgram curves obtained with several different concentrations, using BIA evaluation software package. The fitting of the data followed a suitable binding model using steady state fitting

For FcγRIIB 10,000 resonance units (RU) of a capturing system recognizing a poly-histidine sequence (pentaHis monoclonal antibody, Qiagen Hilden, cat. no. 34660) was immobilized by the use of an amine coupling kit supplied by the GE Healthcare and a CM5 chip at pH 4.5. FcγRIIB was captured at a concentration of 5 μg/ml by with a pulse of 120 sec at a flow of 5 μl/min Different antibodies were passed at a concentration of 1,340 nM with a flow rate of 5 μl/min through the flow cells at 298 K for 60 sec to record the association phase. The dissociation phase was monitored for up to 120 sec and triggered by switching from the sample solution to running buffer. The surface was regenerated by a 0.5 min washing with a glycine pH 2.5 solution at a flow rate of 30 ml/min. For all experiments HBS-P+ buffer supplied by GE Healthcare was chosen (10 mM HEPES, pH 7.4, 150 mM NaCl, 0.05% (v/v) Surfactant P20).

Bulk refractive index differences were corrected for by subtracting the response obtained from a surface without captured FcγRIIB. Blank injections are also subtracted (=double referencing).

Due to the very low intrinsic affinity of FcγRIIB to wild-type IgG1 no affinity was calculated rather a qualitative binding was assessed.

The following tables summarize the effects of introducing a mutation into the Fc part on binding to FcγRI, FcγRIIA, FcγRIIB, and FcγRIIIAV1-58 (A) as well as the effect on ADCC (measured without (BLT) and with target cells (ADCC)) and on C1q binding (B)

TABLE 1a

| | FcγRI | FcγRIIaR131 | FcγRIIIAV158 | FcγRIIB |
|---|---|---|---|---|
| WT IgG1 | ++ (5 nM) | ++ (2 μM) | + (0.7 μM) | ++ |
| IgG4 | − | +/− (10 μM) | − (>20 μM) | + |
| SPLE | | | | |
| IgG1 | ++ (6 nM) | − (>20 μM) | − (>20 μM) | − |
| P329G | | | | |
| IgG1 | ++ (8 nM) | + (4.4 μM) | + (1.8 μM) | + |
| P329A ge | | | | |
| IgG1 | − | − (>2 μM) | − (>20 μM) | |
| P329G | | | | |
| LALA | | | | |
| IgG1 | ++ (10 nM) | − (>20 μM) | − (>10 μM) | − |
| P329G ge | | | | |
| | | | *++ for ge | |
| | | | IgG1 | |
| | | | 30 nM | |

TABLE 1b

| | | | | | | ADCC without | ADCC with |
|---|---|---|---|---|---|---|---|
| Mutant | FcγRI | FcγRII | FcγRIII | C1q | | target cells | target cells |
| Assay | BIAcore | BIAcore | BIAcore | CDC | C1q | BLT | ADCC |
| P329G | + | −− | −− | −− | −− | −− | −− |
| P329R | n.d. | n.d. | n.d. | n.d. | n.d. | −− | −− |
| LALA | − | n.d. | − | − | n.d. | n.d. | −− |
| IgG1_P329G/LALA | −− | −− | −− | n.d. | n.d. | n.d. | n.d. |
| IgG4_SPLE | −− | − | −− | −− | −− | n.d. | n.d. |

−− strongly reduced/inactive in contrast to wt,
− reduced in contrast to wt,
+ comparable to wt interaction,
n.d. not determined/no result.

In more detail the following results have been obtained:

### Affinity to the FcγRI Receptor

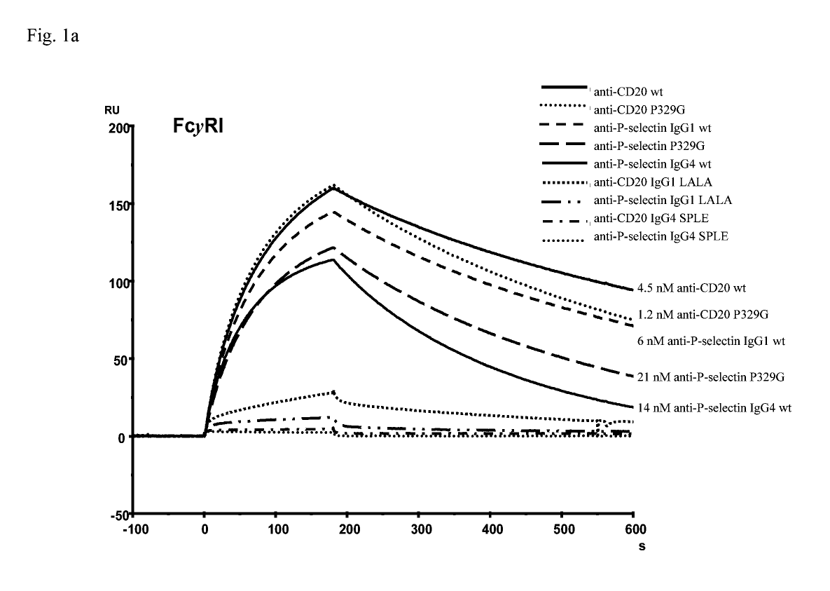

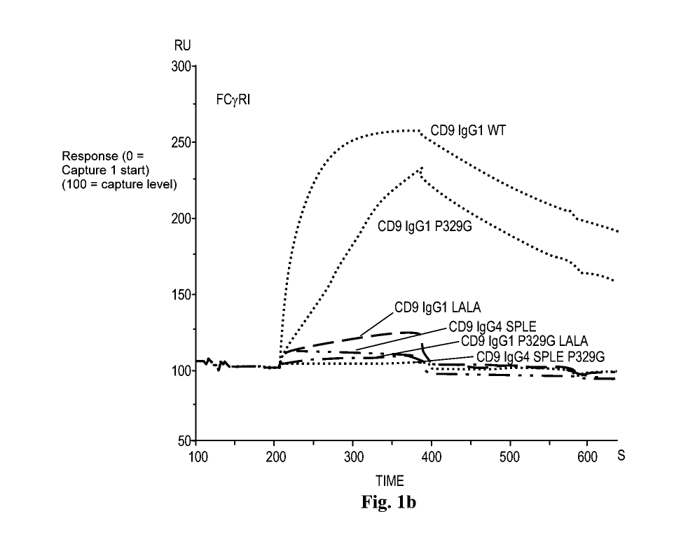

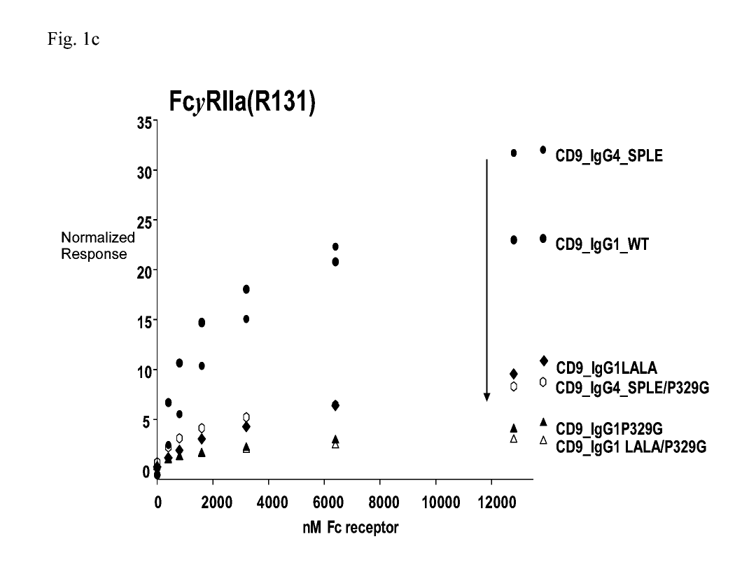

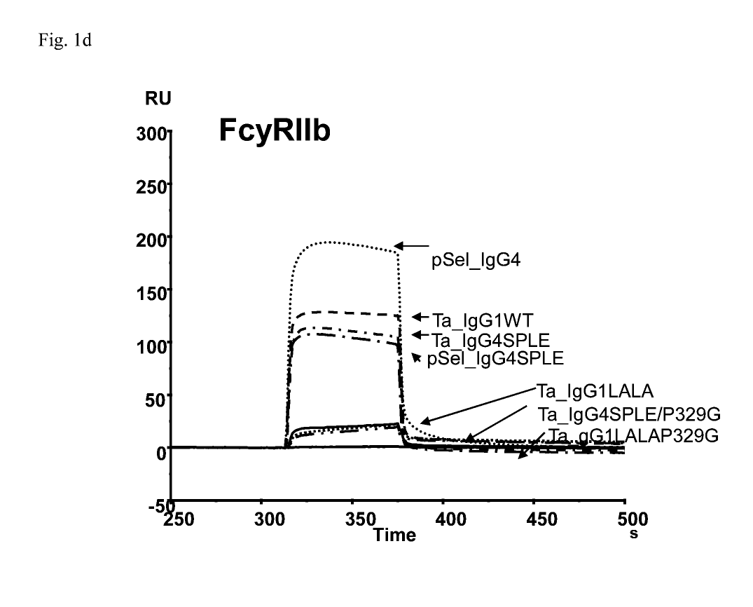

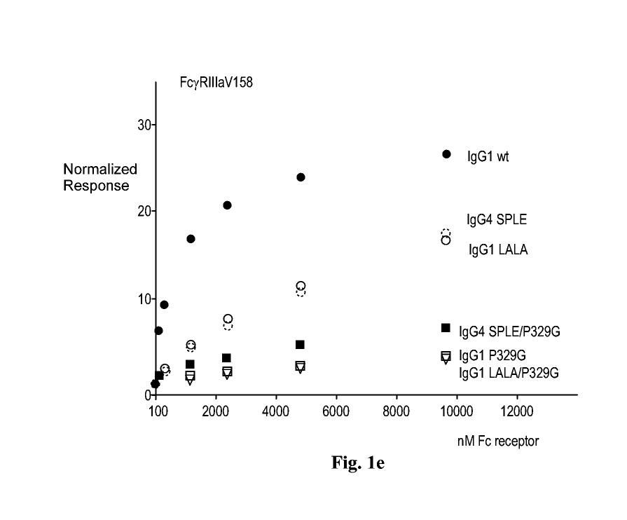

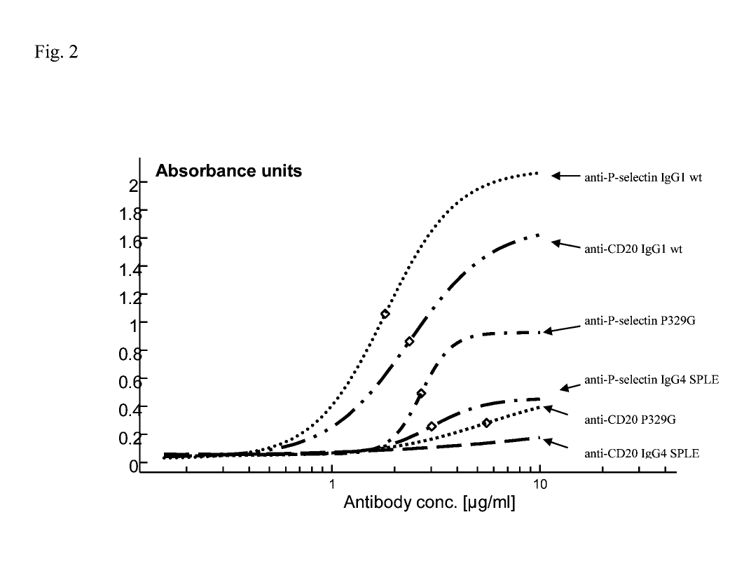

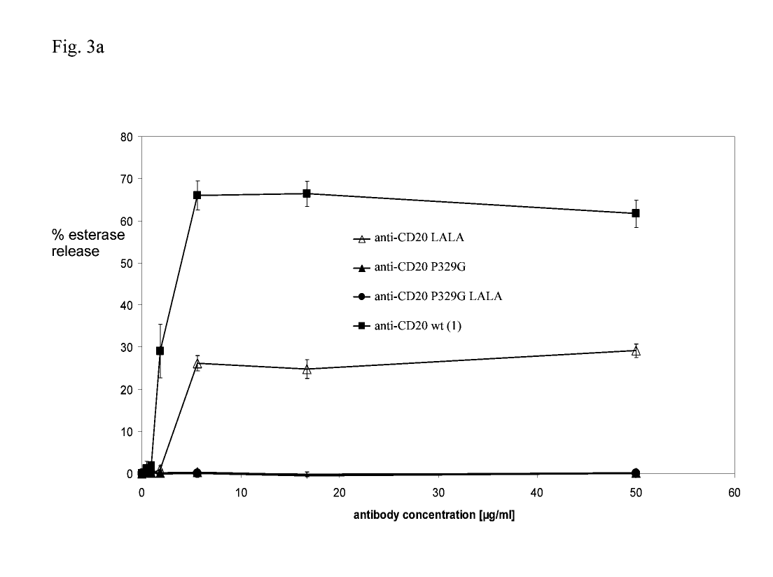

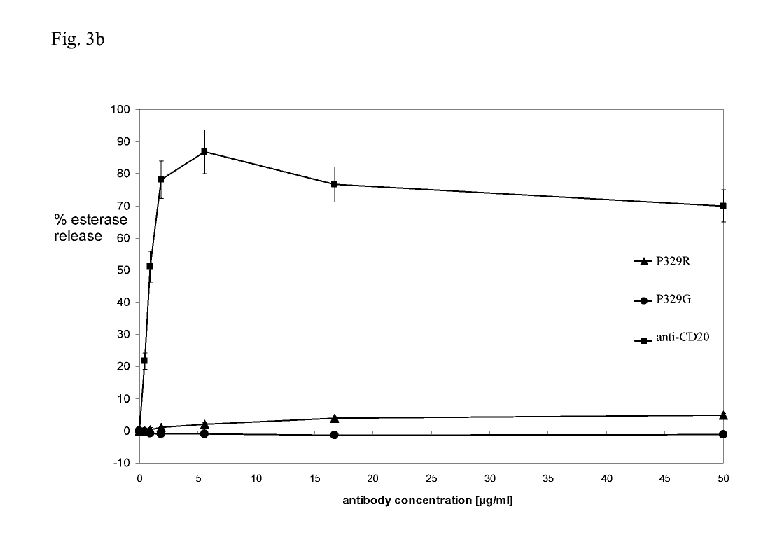

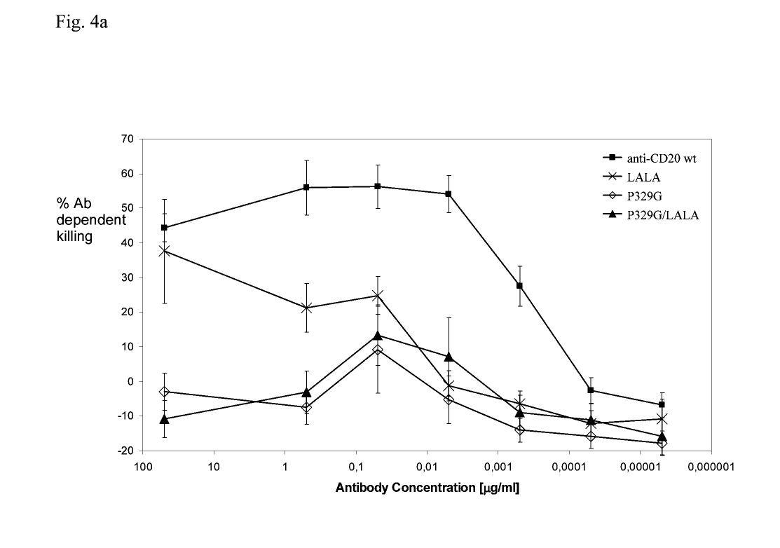

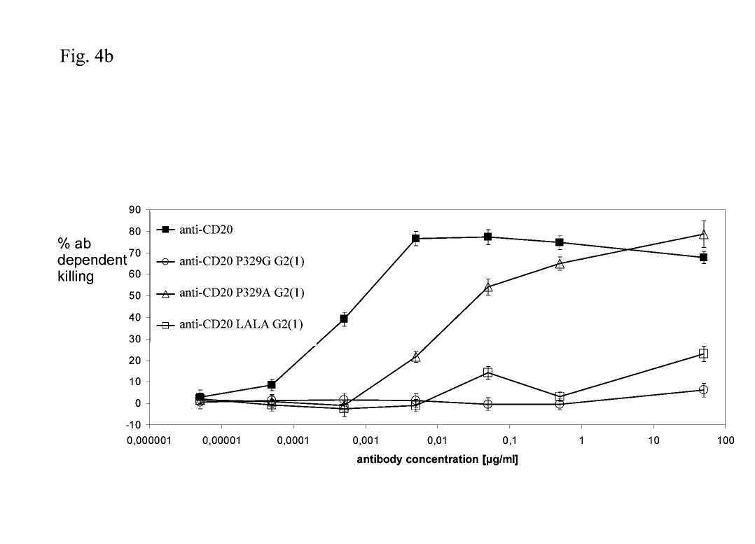

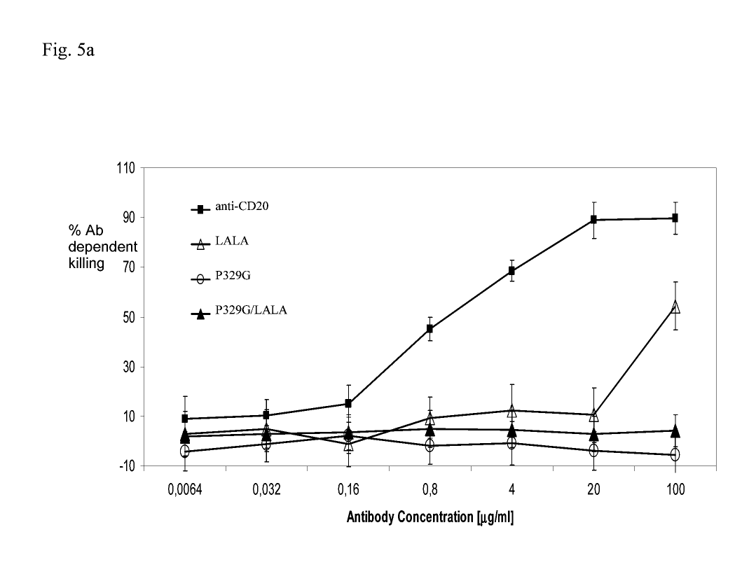

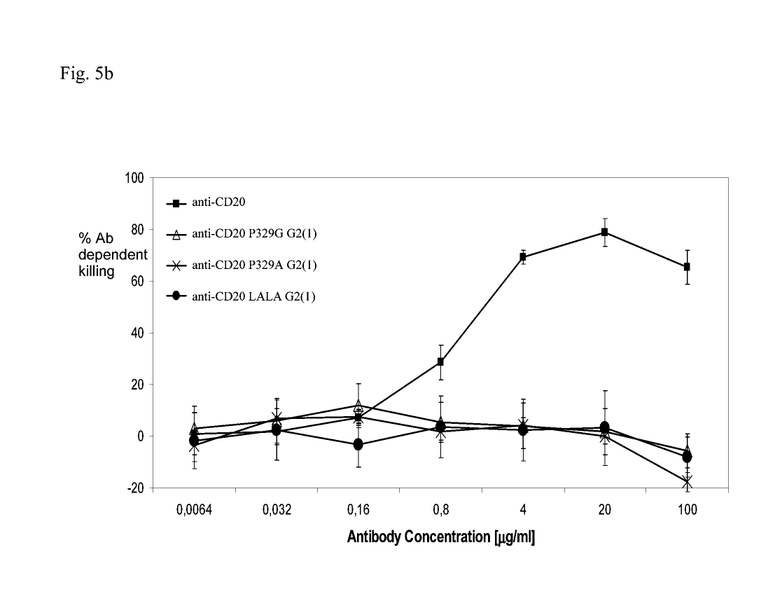

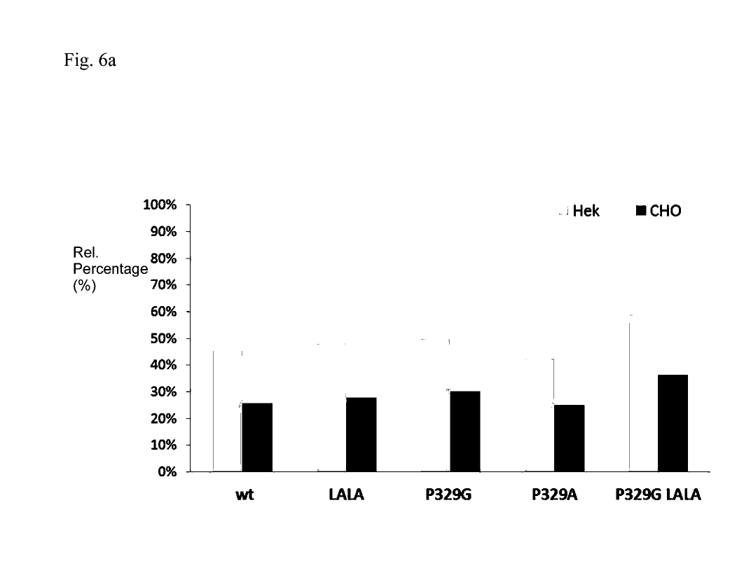

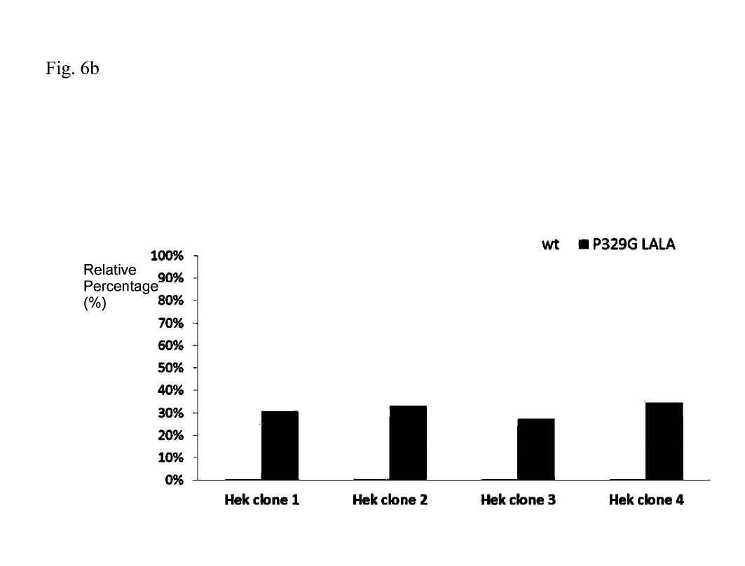

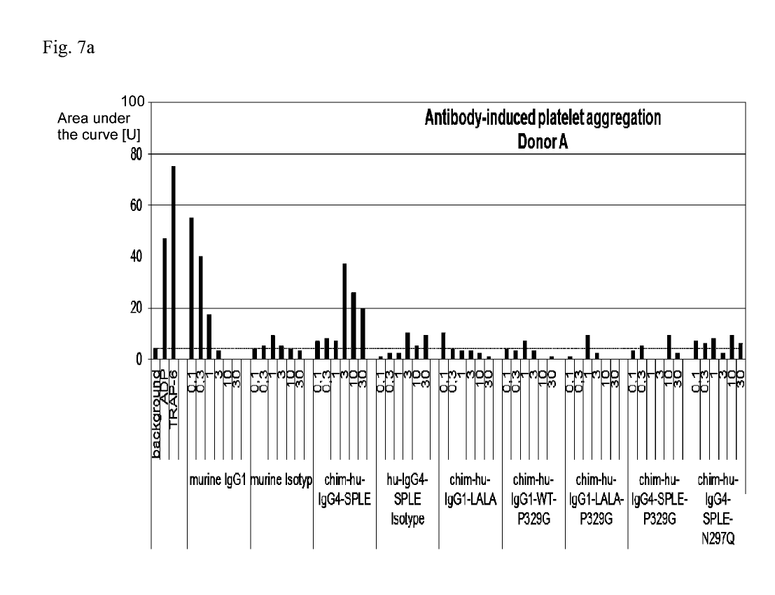

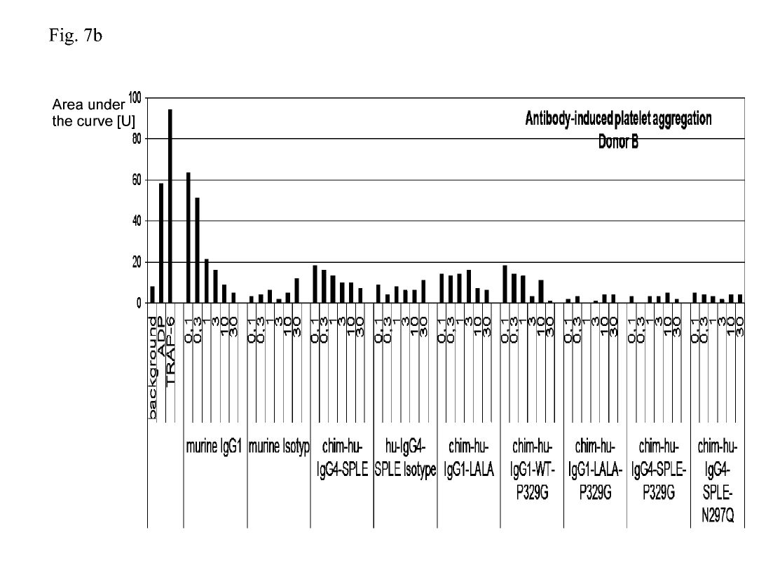

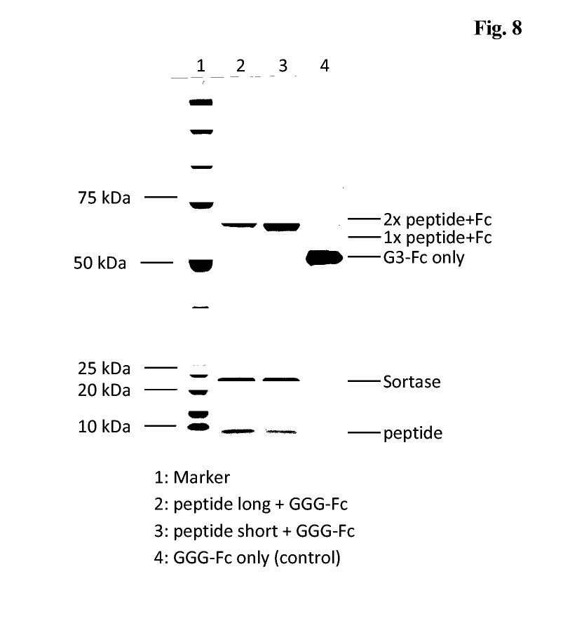

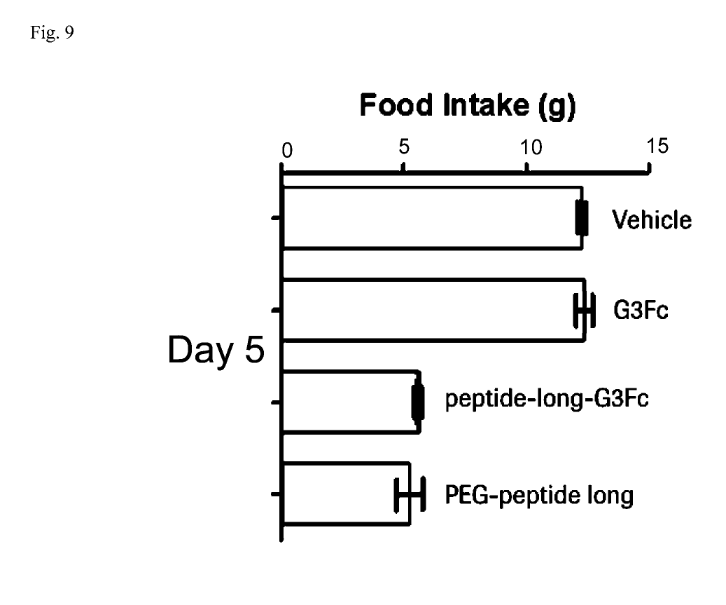

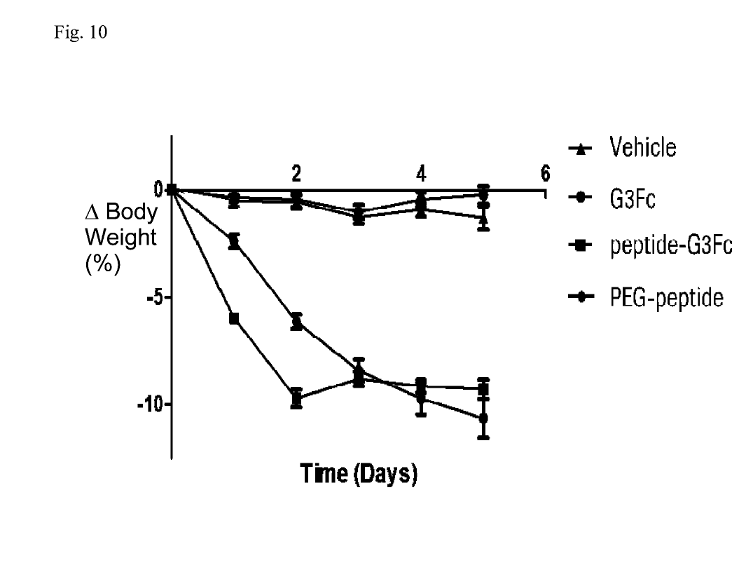

P329G, P329A, SPLE and LALA mutations have been introduced into the Fc polypeptide of a P-selectin, CD20 and CD9 antibody, and the binding affinity to FcγRI was measured with the BIAcore system. Whereas the antibody with the P329G mutation still binds to FcγR1 (FIGS. 1a and 1b), introduction of triple mutations P329G/LALA and P329G/SPLE, respectively, resulted in antibodies for which nearly no binding could be detected (FIG. 1b). The LALA or SPLE mutations decreased binding to the receptor more than P329G alone but less than in combination with P329G (FIGS. 1a and 1b). Thus, the combination of P329G with either LALA or SPLE mutations is much more effective than the P329G mutation or the double mutations LALA or SPLE alone. The kd value for the CD20 IgG1 wild-type antibody was 4.6 nM and for the P329G mutant of the same antibody 5.7 nM, but for the triple mutant P329G/LALA no kd value could be determined due to the nearly undetectable binding of the antibody to the FcγRI receptor. The antibody itself, i.e. whether a CD9 or CD20 or P-selectin was tested, has a minor effect on the binding affinities.

### Affinity to the FcγRIIA Receptor

P329G, SPLE and LALA mutations, respectively, have been introduced into the Fc polypeptide of the CD9 antibody and the binding affinity to the FcγRIIA-R131 receptor was measured with the BIAcore system. Binding level is normalized such as captured mAb represents 100 RU. So not more than approximately 20 RU is expected for a 1:1 stoichiometry. FIG. 1c shows that the binding to the FcγRIIA receptor is strongly reduced by introducing the LALA, SPLE/P329G, P329G and LALA/P329G mutation into the Fc variant. In contrast to binding to the FcγR1 receptor, the introduction of the P329G mutation alone is able to very strongly block the binding to said receptor, more or less to a similar extent as the triple mutation P329G/LALA (FIG. 1c).

### Affinity to the FcγRIIB Receptor

SPLE, LALA, SPLE/P329G and LALA/P329G mutations, respectively, have been introduced into the Fc polypeptide of the CD9 and P-selectin antibody and the binding affinity to FcγRIIB receptor was measured with the BIAcore system. FIG. 1d shows that the binding to the FcγRIIB receptor is strongly reduced in the LALA and triple mutants P329G/LALA, P329G/SPLE

### Affinity to the FcγRIIIA Receptor

P329G, LALA, SPLE, P329G/LALA, and SPLE/P329G mutations have been introduced into the Fc polypeptide of the CD9 and the binding affinity to FcγRIIIA-V158 receptor was measured with the BIAcore system. The P329G mutation and the triple mutation P329G/LALA reduced binding to the FcγRIIIA receptor most strongly, to nearly undetectable levels. The P329G/SPLE also lead to a strongly reduced binding affinity, the mutations SPLE and LALA, respectively, only slightly decreased the binding affinity to the FcγRIIIA receptor (FIG. 1e).

### Example 3

### C1Q ELISA

The binding properties of the different polypeptides comprising Fc variants to C1q were analyzed by an ELISA sandwich type immunoassay. Each variant is coupled to a hydrophobic Maxisorb 96 well plate at 8 concentrations between 10 μg/ml and 0 μg/ml. This coupling simulates complexes of antibodies, which is a prerequisite for high affinity binding of the C1q molecule. After washing, the samples are incubated to allow C1q binding. After further washing the bound C1q molecule is detected by a polyclonal rabbit anti-hC1q antibody. Following the next washing step, an enzyme labeled anti-rabbit-Fcγ specific antibody is added. Immunological reaction is made visible by addition of a substrate that is converted to a colored product by the enzyme. The resulting absorbance, measured photometrically, is proportional to the amount of C1q bound to the antibody to be investigated. EC₅₀ values of the variant-C1q interaction were calculated. The absorption units resulting from the coloring reaction are plotted against the concentration of the antibody. The antibody concentration at the half maximum response determines the EC₅₀ value. This read-out is reported as relative difference to the reference standard measured on the same plate together with the coefficient of variation of sample and reference.

The P329G mutation introduced into the P-selectin or CD20 antibody strongly reduced binding to C1q, similar to the SPLE mutation (FIG. 2). Table 3 summarizes the calculated EC 50 values for binding of the variants to C1q. C1q belongs to the complement activation proteins and plays a major role in the activation of the classical pathway of the complement, which leads to the formation of the membrane attack complex. C1q is also involved in other immunological processes such as enhancement of phagocytosis, clearance of apoptotic cells or neutralization of virus. Thus, it can be expected that the mutants shown here to reduce binding to C1q, e.g. P329G and SPLE, as well as very likely also the triple mutations comprising the aforementioned single mutations, strongly reduces the above mentioned functions of C1q.

TABLE 2

| Antibody | EC₅₀ value |
|---|---|
| P-Selectin IgG1wt | 1.8 |
| anti-CD20 antibody IgG1 wt | 2.4 |
| P-Selectin IgG1 P329G | 2.7 |
| P-Selectin IgG4 SPLE | 3 |
| anti-CD20 antibody IgG1 P329G | 5.5 |
| anti-CD20 antibody IgG4 SPLE | >10 |

### Example 4

### ADCC without Target Cells, BLT Assay

The antibodies to be tested (CD20 and CD9) were coated in PBS over night at 4° C. in suitable 96-flat bottom well plates. After washing the plate with PBS, the remaining binding sites were blocked with PBS/1% BSA solution for 1 h at RT. In the meantime, the effector cells (NK cell line transfected to express low or high affine human FcγRIII) were harvested and 200 000 living cells/well were seeded in 100 μl/well AIM V medium into the wells after discarding the blocking buffer. 100 μl/well saponin buffer (0.5% saponin+1% BSA in PBS) was used to determine the maximal esterase release by the effector cells. The cells were incubated for 3 h at 37° C., 5% CO₂ in an incubator. After 3 h, 20 μl/well of the supernatants were mixed with 180 μl/well BLT substrate (0.2 mM BLT+0.11 mM DTNB in 0.1 M Tris-HCl, pH 8.0) and incubated for 30 min at 37° C. before reading the plate at 405 nm in a microplate reader. The percentage of esterase release was determined setting the maximal release (saponin-treated cells) to 100% and the non-stimulated cells (not antibody coated) to 0% release.

The wild-type anti-CD20 antibody shows strong induction of cytolytic activity. The LALA variant shows a marked reduction in esterase release, whereas the P329G and the P329G/LALA variant do not show any ADCC activity (FIG. 3a). FIG. 3b shows that not only an exchange of G at position P329 leads to markedly reduced cytosolic activity but also an exchange of P329 to R329 (CD20 antibody). Thus arginine appears to destroy the function of the proline sandwich in the antibody, similar to glycine. The strongly reduced ADCC observed here for the P329G mutant most likely resulted from the strongly reduced binding to the FcγRIIA and FcγRIIIA receptor (see FIG. 1c and FIG. 1e).

### Example 5

### ADCC with Target Cells

Human peripheral blood mononuclear cells (PBMC) were used as effector cells and were prepared using Histopaque-1077 (Sigma Diagnostics Inc., St. Louis, Mo. 63178 USA) and following essentially the manufacturer's instructions. In brief, venous blood was taken with heparinized syringes from volunteers. The blood was diluted 1:0.75-1.3 with PBS (not containing Ca²⁺ or Mg²⁺) and layered on Histopaque-1077. The gradient was centrifuged at 400×g for 30 mM at room temperature (RT) without breaks. The interphase containing the PBMC was collected and washed with PBS (50 ml per cells from two gradients) and harvested by centrifugation at 300×g for 10 minutes at RT. After resuspension of the pellet with PBS, the PBMC were counted and washed a second time by centrifugation at 200×g for 10 minutes at RT. The cells were then resuspended in the appropriate medium for the subsequent procedures.

The effector to target ratio used for the ADCC assays was 25:1 and 10:1 for PBMC and NK cells, respectively. The effector cells were prepared in AIM-V medium at the appropriate concentration in order to add 50 ml per well of round bottom 96 well plates. Target cells were human B lymphoma cells (e.g., Raji cells) grown in DMEM containing 10% FCS. Target cells were washed in PBS, counted and resuspended in AIM-V at 0.3 million per ml in order to add 30′000 cells in 100 ml per microwell. Antibodies were diluted in AIM-V, added in 50 ml to the pre-plated target cells and allowed to bind to the targets for 10 minutes at RT. Then the effector cells were added and the plate was incubated for 4 hours at 37° C. in a humidified atmosphere containing 5% CO₂. Killing of target cells was assessed by measurement of lactate dehydrogenase (LDH) release from damaged cells using the Cytotoxicity Detection kit (Roche Diagnostics, Rotkreuz, Switzerland). After the 4-hour incubation the plates were centrifuged at 800×g. 100 ml supernatant from each well was transferred to a new transparent flat bottom 96 well plate. 100 ml color substrate buffer from the kit were added per well. The Vₘₐₓ values of the color reaction were determined in an ELISA reader at 490 nm for at least 10 min using SOFTmax PRO software (Molecular Devices, Sunnyvale, Calif. 94089, USA). Spontaneous LDH release was measured from wells containing only target and effector cells but no antibodies. Maximal release was determined from wells containing only target cells and 1% Triton X-100.

Percentage of specific antibody-mediated killing was calculated as follows: ((x−SR)/(MR−SR)*100, where x is the mean of Vmax at a specific antibody concentration, SR is the mean of Vmax of the spontaneous release and MR is the mean of V. of the maximal release.

The potency to recruit immune-effector cells depends on type of Fc variant as measured by classical ADCC assay. Here, human NK cell-line transfected with human FcγRIIIA was used as effector and CD20 positive Raji cells were used as target cells. As can be seen in FIG. 4a the ADCC is strongly reduced in anti-CD20 antibody Fc variants wherein glycine replaces proline (P329G) and also, to a similar extent, in the double mutant P329G/LALA. In contrast the ADCC decrease was less strong with the LALA mutation. In order to better distinguish between the different variants, the variants were also produced in the glycoengineered version to enhance the ADCC potential. It can be observed that the parental molecule (anti-CD20 antibody) shows strong ADCC as expected. The LALA version is strongly impaired in its ADCC potential. The P329G mutant very strongly decreased the ADCC; much more than a P329A variant of the anti-CD20 antibody (FIG. 4b).

### Example 6

### Complement Activity

Target cells were counted, washed with PBS, resuspended in AIM-V (Invitrogen) at 1 million cells per ml. 50 ml cells were plated per well in a flat bottom 96 well plate. Antibody dilutions were prepared in AIM-V and added in 50 ml to the cells. Antibodies were allowed to bind to the cells for 10 minutes at room temperature. Human serum complement (Quidel) was freshly thawed, diluted 3-fold with AIM-V and added in 50 ml to the wells. Rabbit complement (Cedarlane Laboratories) was prepared as described by the manufacturer, diluted 3-fold with AIM-V and added in 50 ml to the wells. As a control, complement sources were heated for 30 min at 56° C. before addition to the assay. The assay plates were incubated for 2 h at 37° C. Killing of cells was determined by measuring LDH release. Briefly, the plates were centrifuged at 300×g for 3 min. 50 ml supernatant per well were transferred to a new 96 well plate and 50 ml of the assay reagent from the Cytotoxicity Kit (Roche) were added. A kinetic measurement with the ELISA reader determined the Vmax corresponding with LDH concentration in the supernatant. Maximal release was determined by incubating the cells in presence of 1% Triton X-100.

The different Fc variants were analyzed to mediate CDC on SUDH-L4 target cells. The non-glycoengineered anti-CD20 antibody molecule shows clear induction of CDC. The LALA variant shows activity only at the highest concentration, whereas and the P329G and P329G/LALA variants do not show any CDC activity (FIG. 5a). Moreover, the LALA variant as well as the P329G and P329A variants of a glycoengineered anti-CD20 antibody molecule do not show any CDC activity (FIG. 5b).

### Example 7

### Carbohydrate Profile of Human IgG1

The carbohydrate profiles of human IgG1 antibodies containing mutations within the Fc, aimed at abrogating the binding to Fcγ receptors, were analyzed by MALDI/TOF-MS in positive ion mode (neutral oligosaccharides).

Human (h) IgG1 variants were treated with sialidase (QA-Bio) following the manufacturer's instructions to remove terminal sialic acid. The neutral oligosaccharides of hIgG1 were subsequently released by PNGase F (QA-Bio) digestion as previously described (Ferrara, C., et al., Biotech. Bioeng. 93 (2006) 851-861). The carbohydrate profiles were analyzed by mass spectrometry (Autoflex, Bruker Daltonics GmbH) in positive ion mode as previously described (Ferrara, C., et al., Biotech. Bioeng. 93 (2006) 851-861).

The carbohydrate profile of the neutral Fc-associated glycans of human IgG1 is characterized by three major m/z peaks, which can be assigned to fucosylated complex oligosaccharide with none (G0), one (G1) or two (G2) terminal galactose residues.

The carbohydrate profiles of hIgG1 containing mutations within the Fc, aimed at abrogating binding to Fc receptors, were analyzed and compared to that obtained for the wild type antibody. The IgG variants containing one of the mutations within the Fc (P329G, LALA, P329A, P329G/LALA) show similar carbohydrate profiles to the wild type antibody, with the Fc-associated glycans being fucosylated complex oligosaccharides (data not shown). Mutation within the Fc can affect the level of terminal galactosylation and terminal sialidation, as observed by replacing amino acid at positions 241, 243, 263, 265, or 301 by alanine (Lund, J., et al., J. Immunol. 157 (1996) 4963-4969).

FIG. 6a shows the relative percentage of galactosylation for the different hIgG1 Fc-variants described here. Slight variations can be observed when the antibodies are expressed in a different host, but no significant difference in terminal galactosylation could be observed.

FIG. 6b indicates the variability in galactosylation content for wild type and IgG1-P329G/LALA for 4 different antibodies, where four different V-domains were compared for their amount of galactosylation when expressed in Hek293 EBNA cells.

### Example 8

### Antibody-Induced Platelet Aggregation in Whole Blood Assay

Whole blood platelet aggregation analysis using the multiplate instrument from Dynabyte. First, 20 ml blood from normal human donors are withdrawn and transferred into hirudine tubes (Dynabyte Medical, #MP0601). Plug minicell impedance device (Dynabead #MP0021) into the multiplate instrument was used for the assay. Then, 175 μl 0.9% NaCl were added to the minicell. Antibody was added to minicell to obtain the final test concentration. Then, 175 μl human blood was added and incubated for 3 min at 37° C. Automated start of impedance analysis for additional 6 min. at 37° C. The data were analyzed by quantification of area-under-the-curve as a measure of platelet aggregation.

The anti-CD9 antibody has been shown to induce platelet activation and platelet aggregation (Worthington, et al., Br. J. Hematol. 74(2) (1990) 216-222). Platelet aggregation induced by antibodies binding to platelets previously has been shown to involve binding to FcγRIIA (de Reys, et al., Blood 81 (1993) 1792-1800). As shown above the mutations LALA, P329G, P329G/LALA and P329G/SPLE introduced into the anti-CD9 antibody strongly reduced binding of the anti-CD9 antibody to the FcγRIIA receptor (FIG. 1c).

The activation (measured by Ca-efflux, data not shown) as well as platelet aggregation induced by an anti-CD9 antibody was eliminated by introducing the P329G and LALA triple mutation into the antibody such that the FcγRIIA binding is strongly reduced compared to the wild-type antibody (see FIGS. 7a and 7b). Murine IgG1 induced platelet aggregation at low antibody concentrations (0.1-1 μ/ml). At higher concentrations overstimulation of platelets leads to silencing of the aggregation response (3-30 μg/ml). Donor variability was observed with chim-hu-IgG4-SPLE. In FIG. 6a data for a chim-hu-IgG4-SPLE responder at higher antibody concentrations and in FIG. 6b data for a chim-hu-IgG4-SPLE non-responder is shown. None of the blood samples showed any aggregation response with the antibody variants chim-hu-IgG1-LALA, chim-hu-IgG-WT-P329G, chim-hu-IgG 1-LALA-P329G, chim-hu-IgG4-SPLE-P329G, and chim-hu-IgG4-SPLE-N297Q. Controls: spontaneous aggregation in untreated blood sample (background); ADP-induced (ADP) and Thrombin analogue-induced (TRAP6) platelet aggregation. Isotype controls: Murine IgG1 (murine Isotype) and human IgG4-SPLE (hu-IgG4-SPLE Isotype).

One possible interpretation of these data is that the decreased binding of the anti-CD9 antibody with the triple mutations to the FcγRIIA receptor is the reason for the diminished platelet aggregation observed with these kinds of mutant antibodies. In principle, prevention of thrombocyte aggregation, as a toxic side-effect of an antibody treatment, might thus be possible by introducing the above mentioned mutations, capable of reducing binding to the FcγRIIA receptor, into the Fc part of an antibody.

### Example 9

### Sortase A Conjugation of Fc-Region and Incretin Receptor Ligand Polypeptide

G3-Fc:
(SEQ ID NO: 68)
GGGDKTHTCPPCPAPELLGGPSVFLEPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.
G4S3-Fc:
(SEQ ID NO: 69)
GGGGSGGGGSGGGGSDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGK.
long peptide:
(SEQ ID NO: 70)
Y-Aib-EGTFTSDYSIYLDKQAA-Aib-EFVAWLLAGGPSSGAPPPSKLP
ETGGSGS-amide
short peptide:
(SEQ ID NO: 71)
Y-Aib-EGTFTSDYSIYLDKQAA-Aib-EFVAWLLAGGGLPETGGSGS-
amide.

For the sortase-mediated transpeptidation reaction, N-terminally truncated Staphylococcus aureus Sortase A was used (Δ₁₋₅₉). The reaction was performed in a buffer containing 50 mM Tris-HCl, 150 mM NaCl, 5 mM CaCl, pH 7.5 (Sortase-buffer). In the reaction, a chemically synthesized peptide bearing a sortase motif at its C-terminus (LPETGGSGS, SEQ ID NO: 72) and an Fc-region bearing an oligo-glycine motif at its N-terminus were linked, resulting in the connecting sequence peptide-LPETGGG-heavy chain Fc-region. To perform the reaction, all reagents were brought in solution in sortase buffer. In a first step, GGG-Fc and peptide were mixed, and the reaction was started by the following addition of Sortase A. The components were mixed by pipetting or vortexing and incubated at 37° C. for 1 h and 24 h, depending on the peptide. Subsequently, the ligation product was purified directly after the transpeptidation reaction, or the reaction was stopped by freezing of the reaction mixture and storage at −20° C. until purification.

Molar ratio peptide:Fc:sortase=10:8:1

### Results

Both long and short synthetic peptides were coupled via sortase mediated transpeptidation to IgG-Fc fragments bearing either a short tri-glycine motif or a longer GGGGSGGGGSGGGGS (SEQ ID NO: 62) sequence at the N-terminus, respectively. Combinations are displayed in Table 3.

TABLE 3
Conjugation of Fc-regions with peptide

| | | | | | conc. | conc. | conc. |
|---|---|---|---|---|---|---|---|
| | Fc | peptide | time | temp | SrtA | Fc | peptide |
| 1 | G3-Fc | long | 3 h | 37° C. | 10 μmol/l | 12.5 μmol/l | 100 μmol/l |
| 2 | G3-Fc | short | 24 h | 37° C. | 10 μmol/l | 12.5 μmol/l | 100 μmol/l |
| 3 | G4S3-Fc | long | 3 h | 37° C. | 10 μmol/l | 12.5 μmol/l | 100 μmol/l |
| 4 | G4S3-Fc | short | 24 h | 37° C. | 10 μmol/l | 12.5 μmol/l | 100 μmol/l |

The Fc-region-incretin receptor ligand polypeptide conjugates had the amino acid sequences of SEQ ID NOs: 95-98 for Long Peptide-G3-Fc, Short Peptide-G3-Fc, Long Peptide G453-Fc, and Short Peptide G453-Fc, respectively.

### Analysis of Sortase-Mediated Transpeptidation

Aliquots of the transpeptidation reactions were analyzed by SDS-PAGE. An example is displayed in FIG. 8, showing the results of conjugation of long or short peptide to G3-Fc. From the gel the efficiency of ligation was estimated densitometrically. As shown in Table 4 about 5% of Fc was not conjugated with peptide while around 90% of Fc was conjugated with two peptide moieties.

TABLE 4
Efficiency of sortase-mediated transpeptidation
of peptides with G3-Fc

| | long peptide | short peptide |
|---|---|---|
| 2x peptide + G3-Fc [%] | 87.00 | 90.75 |
| 1x peptide + G3-Fc [%] | 6.91 | 6.51 |
| non-ligated G3-Fc [%] | 6.09 | 2.73 |

The biological activity of the different conjugates is shown in Table 5.

TABLE 5
in vitro efficacy of peptide-Fc fusion molecules
generated by sortase-mediated transpeptidation

| | GIP-RE | GLP1-RE |
|---|---|---|
| Compound | C₅₀ [nM] | C₅₀ [nM] |
| GIP | 0.058 | — |
| GLP1 | — | 0.005 |
| PEG-peptide long | 7.508 | 3.893 |
| G3Fc only | >600 | >600 |
| peptide-long-G3Fc | 1.022 | 0.263 |
| peptide-short-G3Fc | 1.921 | 0.697 |

### Example 10

### Cyclic AMP Assay

The following materials were used: cAMP Hunter™ CHO-K1 GLP-1 or GIP cell lines (DiscoveRx Corporation), Ham's F-12 (Gibco Cat. #21765), 10% heat inactivated FBS (Gibco Cat #16000), Penicillin/Streptomycin/L-Glutamine (Gibco Cat #10378) and 800 μg/ml G418 (geneticin, Gibco Cat. #10131).

CHO-K1 cells expressing GLP-1 or GIP receptors were suspended in 10 ml assay buffer (Krebs-Ringer bicarbonate buffer (Sigma-Aldrich Cat. #K4002) containing 0.5 mM IBMX (Sigma-Aldrich Cat #17018) and 0.1% BSA (Sigma-Aldrich Cat. #A-2153)) at a cell density of 100,000 cells/ml. The cell suspension (25 μl) was subsequently transferred to a half-area plate (Costar Cat. #3694) and drug solutions (25 μl) were added to the wells at appropriate concentrations. The cells were incubated for 30 min at room temperature on a plate shaker. The cAMP content was determined using the Cisbio “cAMP dynamic kit” following the manufacturer's instructions (Cisbio Bioassays, France). All experiments were performed in duplicates and drugs were tested at least twice (N≧2).

### Example 11

### Acute DIO Mouse Studies

Male C57Bl/6 mice (age about 7 month; Jackson laboratories (Bar Harbor, Me., USA)) were housed in a temperature and humidity controlled environment with a 12 h light:12 h dark cycle. The mice were given ad libitum access to water and a high fat chow diet (HFD; 58% of dietary kcal as fat with sucrose, Research Diets D12331) and water starting at 8 weeks age, and access was maintained throughout the study. The mice were sorted by body weight and food intake prior to start of the treatment period and housed four animals per cage. Mice were acclimated at least 6 days before use. The mice were dosed once prior to onset of dark cycle with vehicle (s.c.), control (human IgG1-Fc; s.c.) or compounds (20 nmol/kg, s.c. of either peptide-long-G3Fc or peptide-short-G3Fc). Thereafter body weight and food intake monitored daily for 5 days (N=8 mice/group).

### Data Analysis:

All data shown are the mean±standard error (s.e.m.). Statistical evaluation of the data was carried out using one-way ANOVA, followed by Dunnett's test to determine where statistically significant differences existed between vehicle and drug treated groups. Differences were considered statistically significant at P<0.05. Data analysis was carried out with GraphPad software (GraphPad Prism).

### Results:

A single administration of the compounds peptide-long-G3Fc and peptide-short-G3Fc (20 nmol/kg, s.c.) in male DIO mice induced a significant decrease in body weight gain versus vehicle-treated animals and reduced cumulative food intake (FIG. 9 and FIG. 10).

### Example 12

### Acute db/db Mouse Studies

Ten week-old male db/db mice (C57BLKS; BKS. Cg-m+/+Lepr (000642); Jackson Laboratories, USA) were housed in a temperature and humidity controlled environment with a 12 h light:12 h dark cycle, and given access to normal chow and water ad libitum (chow, 5% kcal as fat, Harlan 7912). The mice (˜42 g) were randomized to various treatment groups based on ad libitum blood glucose levels. The mice were administered vehicle (s.c.), control (s.c.) or the compounds (20 nmol/kg, s.c.) prior to the onset of the dark cycle. The following day, the mice were fasted for 6 h prior to an intraperitoneal glucose challenge test (N=8 mice/group). Blood samples were collected from tail clips following a 6 h fast, for determination of baseline values (t=0 min.), using a handheld FreeStyle Freedom Lite glucose meter (Abbott). The mice were then injected with an intraperitoneal bolus of glucose (1 g/kg; 25% dextrose solution), and additional blood samples were collected at regular intervals (t=15, 30, 60 and 120 min) for glucose measurement. To analyze the effects of the compounds on intraperitoneal glucose tolerance the area under the curve (AUC_{0-120 min}) for blood glucose excursion was determined using the trapezoid method.

### Data Analysis:

All data shown are the mean±standard error (s.e.m.). Statistical evaluation of the data was carried out using one-way ANOVA, followed by Dunnett's test to determine where statistically significant differences existed between vehicle and drug treated groups. Differences were considered statistically significant at P<0.05. Data analysis was carried out with GraphPad software (GraphPad Prism).

### Results:

An acute administration of the compounds peptide-long-G3Fc and peptide-short-G3Fc (20 nmol/kg, s.c.) to male db/db mice significantly decreased glucose excursion in response to an intraperitoneal glucose challenge (ipGTT; AUC ipGTT). The effect is dose-dependent.

1. An Fc-region conjugate comprising one, two, three, or four naturally occurring or synthetic incretin receptor ligand polypeptides each covalently linked to an Fc-region, wherein the conjugate comprises the amino acid sequence LPETG.

2. The Fc-region conjugate according to claim 1, characterized in that the Fc-region is a human Fc-region with a mutation of the amino acid residue at position 329 and at least one further mutation of at least one amino acid selected from the group comprising amino acid residues at position 228, 233, 234, 235, 236, 237, 297, 318, 320, 322 and 331 to a different residue, wherein the residues in the Fc-region are numbered according to the EU index of Kabat.

3. The Fc-region conjugate according to claim 1, characterized in that the variant human Fc-region has a reduced affinity to the human FcγRIIIA and/or FcγRIIA and/or FcγRI compared to an Fc-region fusion polypeptide or conjugate comprising a wild-type IgG Fc-region.

4. The Fc-region conjugate according to claim 2, characterized in that the at least one further mutation of at least one amino acid in the Fc-region is S228P, E233P, L234A, L235A, L235E, N297A, N297D, or P331S.

5. The Fc-region conjugate according to claim 4, characterized in that the at least one further mutation in the Fc-region is L234A and L235A if the Fc-region is of human IgG1 isotype or S228P and L235E if the Fc-region is of human IgG4 isotype.

6. The Fc-region conjugate according to claim 1, characterized in that thrombocyte aggregation induced by the Fc-region fusion polypeptide or conjugate is reduced compared to the thrombocyte aggregation induced by an Fc-region fusion polypeptide or conjugate comprising a wild-type human IgG Fc-region.

7. The Fc-region conjugate according to claim 1, characterized in comprising one or two incretin receptor ligand polypeptides.

8. The Fc-region conjugate according to claim 1, characterized in that each of the incretin receptor ligand polypeptides is fused or conjugated to the N-terminus of one Fc-region polypeptide chain, whereby each Fc-region polypeptide chain is fused or conjugated only to one incretin receptor ligand polypeptide.

9. The Fc-region conjugate according to claim 7, characterized in that each of the incretin receptor ligand polypeptides is fused or conjugated to the C-terminus of one Fc-region polypeptide chain, whereby each Fc-region polypeptide chain is fused or conjugated only to one incretin receptor ligand polypeptide.

10. The Fc-region conjugate according to claim 1, characterized in that the incretin receptor ligand polypeptides are selected independently from each other from GIP, GLP-1, exendin-3, exendin-4, dual GIP-GLP-1 agonists, triple GIP-GLP-1-glucagon receptor agonists, chimeric GIP/GLP agonists, and precursors, derivatives, or functional fragments thereof.

11. Use of an Fc-region conjugate according to claim 1 as a medicament.

12. Use of an Fc-region conjugate according to claim 1 for the manufacture of a medicament for the treatment of a disease, wherein it is favorable that the effector function of the fusion polypeptide or conjugate comprising a variant Fc-region of a wild-type human IgG Fc-region is reduced compared to the effector function induced by a fusion polypeptide or conjugate comprising a wild-type human IgG Fc-region.

13. Use of an Fc-region conjugate according to claim 2 comprising a variant Fc-region of a wild-type human IgG Fc-region, wherein Pro329 of the wild-type human IgG Fc-region is substituted with glycine, wherein the residues are numbered according to the EU index of Kabat, wherein the fusion polypeptide or conjugate exhibits a reduced affinity to the human FcγRIIIA and FcγRIIA for down-modulation of ADCC by at least 20% of the ADCC induced by a fusion polypeptide or conjugate comprising the wild-type human IgG Fc-region, and/or for down-modulation of ADCP.

14. The use according to claim 11, characterized in that the disease is type-2 diabetes or obesity.

15. The use according to claim 11, characterized in that the disease is type-1 diabetes.


SEQUENCE LISTING (98 sequences)

SEQ ID NO: 1 - protein, 31 aa, Artificial Sequence
  GLP-1(7-37)
HAEGTFTSDVSSYLEGQAAKEFIAWLVKGRG

SEQ ID NO: 2 - protein, 30 aa, Artificial Sequence
  GLP-1(7-36)
HAEGTFTSDVSSYLEGQAAKEFIAWLVKGR

SEQ ID NO: 3 - protein, 39 aa, Artificial Sequence
  exendin-3
HSDGTFTSDLSKQMEEEAVRLFIEWLKNGGPSSGAPPPS

SEQ ID NO: 4 - protein, 39 aa, Artificial Sequence
  exendin-4
HGEGTFTSDLSKQMEEEAVRLFIEWLKNGGPSSGAPPPS

SEQ ID NO: 5 - protein, 31 aa, Artificial Sequence
  exendin-4(1-31) desGlu(17) Tyr(32)
HGEGTFTSDLSKQMEEAVRLFIEWLKNGGPY

SEQ ID NO: 6 - protein, 31 aa, Artificial Sequence
  exendin-4(1-30) Tyr(31)
HGEGTFTSDLSKQMEEEAVRLFIEWLKNGGY

SEQ ID NO: 7 - protein, 31 aa, Artificial Sequence
  exendin-4(9-39)
DLSKQMEEEAVRLFIEWLKNGGPSSGAPPPS

SEQ ID NO: 8 - protein, 19 aa, Artificial Sequence
  incretin receptor ligand polypeptide
  MISC_FEATURE (17)..(17): Xaa denotes Lys or Arg
SYLEGQAAKEFIAWLVXGR

SEQ ID NO: 9 - protein, 20 aa, Artificial Sequence
  incretin receptor ligand polypeptide
  MISC_FEATURE (18)..(18): Xaa denotes Lys or Arg
SSYLEGQAAKEFIAWLVXGR

SEQ ID NO: 10 - protein, 21 aa, Artificial Sequence
  incretin receptor ligand polypeptide
  MISC_FEATURE (19)..(19): Xaa denotes Lys or Arg
VSSYLEGQAAKEFIAWLVXGR

SEQ ID NO: 11 - protein, 22 aa, Artificial Sequence
  incretin receptor ligand polypeptide
  MISC_FEATURE (20)..(20): Xaa denotes Lys or Arg
DVSSYLEGQAAKEFIAWLVXGR

SEQ ID NO: 12 - protein, 23 aa, Artificial Sequence
  incretin receptor ligand polypeptide
  MISC_FEATURE (21)..(21): Xaa denotes Lys or Arg
SDVSSYLEGQAAKEFIAWLVXGR

SEQ ID NO: 13 - protein, 24 aa, Artificial Sequence
  incretin receptor ligand polypeptide
  MISC_FEATURE (22)..(22): Xaa denotes Lys or Arg
TSDVSSYLEGQAAKEFIAWLVXGR

SEQ ID NO: 14 - protein, 25 aa, Artificial Sequence
  incretin receptor ligand polypeptide
  MISC_FEATURE (23)..(23): Xaa denotes Lys or Arg
FTSDVSSYLEGQAAKEFIAWLVXGR

SEQ ID NO: 15 - protein, 26 aa, Artificial Sequence
  incretin receptor ligand polypeptide
  MISC_FEATURE (24)..(24): Xaa denotes Lys or Arg
TFTSDVSSYLEGQAAKEFIAWLVXGR

SEQ ID NO: 16 - protein, 27 aa, Artificial Sequence
  incretin receptor ligand polypeptide
  MISC_FEATURE (25)..(25): Xaa denotes Lys or Arg
GTFTSDVSSYLEGQAAKEFIAWLVXGR

SEQ ID NO: 17 - protein, 28 aa, Artificial Sequence
  incretin receptor ligand polypeptide
  MISC_FEATURE (26)..(26): Xaa denotes Lys or Arg
EGTFTSDVSSYLEGQAAKEFIAWLVXGR

SEQ ID NO: 18 - protein, 29 aa, Artificial Sequence
  incretin receptor ligand polypeptide
  MISC_FEATURE (27)..(27): Xaa denotes Lys or Arg
AEGTFTSDVSSYLEGQAAKEFIAWLVXGR

SEQ ID NO: 19 - protein, 30 aa, Artificial Sequence
  incretin receptor ligand polypeptide
  MISC_FEATURE (28)..(28): Xaa denotes Lys or Arg
HAEGTFTSDVSSYLEGQAAKEFIAWLVXGR

SEQ ID NO: 20 - protein, 31 aa, Artificial Sequence
  incretin receptor ligand polypeptide
  MISC_FEATURE (29)..(29): Xaa denotes Lys or Arg
HDAEGTFTSDVSSYLEGQAAKEFIAWLVXGR

SEQ ID NO: 21 - protein, 39 aa, Artificial Sequence
  hybrid GLP-1/exendin polypeptide
HAEGTFTSDVSSYLEGQAAKEFIAWLVKGRPSSGAPPPS

SEQ ID NO: 22 - protein, 37 aa, Artificial Sequence
  incretin receptor ligand polypeptide
HDEFERHAEGTFTSDVSSYLEGQAAKEFIAWLVKGRK

SEQ ID NO: 23 - protein, 31 aa, Artificial Sequence
  incretin receptor ligand polypeptide
HAEGTFTSDVSSYLEGQAAKEFIAWLVKGRK

SEQ ID NO: 24 - protein, 40 aa, Artificial Sequence
  incretin receptor ligand polypeptide
HGEGTFTSDLSKQMEEEAVRLFIEWLKNGGPSSGAPPPSK

SEQ ID NO: 25 - protein, 40 aa, Artificial Sequence
  incretin receptor ligand polypeptide
HSDGTFTSDLSKQMEEEAVRLFIEWLKNGGPSSGAPPPSK

SEQ ID NO: 26 - protein, 31 aa, Artificial Sequence
  incretin receptor ligand polypeptide
HGEGTFTSDLSKEMEEEVRLFIEWLKNGGPY

SEQ ID NO: 27 - protein, 30 aa, Artificial Sequence
  incretin receptor ligand polypeptide
HGEGTFTSDLSKEMEEEVRLFIEWLKNGGY

SEQ ID NO: 28 - protein, 29 aa, Artificial Sequence
  incretin receptor ligand polypeptide
DLSKQMEEEAVRLFIEWLKGGPSSGPPPS

SEQ ID NO: 29 - protein, 40 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVCWLLAGGPSSGAPPPSK

SEQ ID NO: 30 - protein, 40 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVNWLLAGGPSSGAPPPSK

SEQ ID NO: 31 - protein, 40 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVAWLLAGGPSSGAPPPSK

SEQ ID NO: 32 - protein, 31 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVNWLLAGGG

SEQ ID NO: 33 - protein, 31 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVAWLLAGGG

SEQ ID NO: 34 - protein, 40 aa, Artificial Sequence
  incretin receptor ligand polypeptide
  MISC_FEATURE (2)..(2): Xaa denotes aib
YXEGTFTSDYSIYLDEQAAKEFVNWLLAGGPSSGAPPPSC

SEQ ID NO: 35 - protein, 40 aa, Artificial Sequence
  incretin receptor ligand polypeptide
  MISC_FEATURE (2)..(2): Xaa denotes aib
  MISC_FEATURE (20)..(20): Xaa denotes aib
YXEGTFTSDYSIYLDKQAAXEFVNWLLAGGPSSGAPPPSC

SEQ ID NO: 36 - protein, 40 aa, Artificial Sequence
  incretin receptor ligand polypeptide
  MISC_FEATURE (2)..(2): Xaa denotes aib
  MISC_FEATURE (20)..(20): Xaa denotes aib
YXQGTFTSDYSIYLDKQAAXEFVNWLLAGGPSSGAPPPSK

SEQ ID NO: 37 - protein, 40 aa, Artificial Sequence
  incretin receptor ligand polypeptide
  MISC_FEATURE (2)..(2): Xaa denotes aib
  MISC_FEATURE (16)..(20): lactam ring between residues 16 and 20
YXQGTFTSDYSIYLDEQAAKEFVNWLLAGGPSSGAPPPSC

SEQ ID NO: 38 - protein, 40 aa, Artificial Sequence
  incretin receptor ligand polypeptide
  MISC_FEATURE (2)..(2): Xaa denotes aib
  MISC_FEATURE (16)..(20): lactam ring between residues 16 and 20
YXQGTFISDYSIYLDEQAAKEFVNWLLAGGPSSGAPPPSC

SEQ ID NO: 39 - protein, 29 aa, Artificial Sequence
  incretin receptor ligand polypeptide
  MISC_FEATURE (2)..(2): Xaa denotes aib
  MISC_FEATURE (16)..(20): lactam ring between residues 16 and 20
YXQGTFISDYSIYLDEQAAKEFVCWLLAG

SEQ ID NO: 40 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 41 - protein, 106 aa, Homo sapiens [sequence omitted]

SEQ ID NO: 42 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 227 aa, Artificial Sequence
  variant human Fc-region of the IgG1 isotype with the mutations L234A, L235A
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 227 aa, Artificial Sequence
  variant human Fc-region of the IgG1 isotype with a hole mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 227 aa, Artificial Sequence
  variant human Fc-region of the IgG1 isotype with a knob mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 227 aa, Artificial Sequence
  variant human Fc-region of the IgG1 isotype with a L234A, L235A and hole mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 227 aa, Artificial Sequence
  variant human Fc-region of the IgG1 isotype with a L234A, L235A and knob mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 227 aa, Artificial Sequence
  variant human Fc-region of the IgG1 isotype with a P329G mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 227 aa, Artificial Sequence
  variant human Fc-region of the IgG1 isotype with a L234A, L235A and P329G mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 227 aa, Artificial Sequence
  variant human Fc-region of the IgG1 isotype with a P239G and hole mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 227 aa, Artificial Sequence
  variant human Fc-region of the IgG1 isotype with a P329G and knob mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 227 aa, Artificial Sequence
  variant human Fc-region of the IgG1 isotype with a L234A, L235A, P329G and hole mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 227 aa, Artificial Sequence
  variant human Fc-region of the IgG1 isotype with a L234A, L235A, P329G and knob mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 229 aa, Artificial Sequence
  variant human Fc-region of the IgG4 isotype with a S228P and L235E mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 229 aa, Artificial Sequence
  variant human Fc-region of the IgG4 isotype with a S228P, L235E and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 12 aa, Artificial Sequence
  linker polypeptide
GGGSGGGSGGGS

SEQ ID NO: 58 - protein, 16 aa, Artificial Sequence
  linker polypeptide
GGGSGGGSGGGSGGGS

SEQ ID NO: 59 - protein, 20 aa, Artificial Sequence
  linker polypeptide
GGGSGGGSGGGSGGGSGGGS

SEQ ID NO: 60 - protein, 24 aa, Artificial Sequence
  linker polypeptide
GGGSGGGSGGGSGGGSGGGSGGGS

SEQ ID NO: 61 - protein, 10 aa, Artificial Sequence
  linker polypeptide
GGGGSGGGGS

SEQ ID NO: 62 - protein, 15 aa, Artificial Sequence
  linker polypeptide
GGGGSGGGGSGGGGS

SEQ ID NO: 63 - protein, 20 aa, Artificial Sequence
  linker polypeptide
GGGGSGGGGSGGGGSGGGGS

SEQ ID NO: 64 - protein, 25 aa, Artificial Sequence
  linker polypeptide
GGGGSGGGGSGGGGSGGGGSGGGGS

SEQ ID NO: 65 - protein, 12 aa, Artificial Sequence
  linker polypeptide
GGGGGSGGGGGS

SEQ ID NO: 66 - protein, 18 aa, Artificial Sequence
  linker polypeptide
GGGGGSGGGGGSGGGGGS

SEQ ID NO: 67 - protein, 24 aa, Artificial Sequence
  linker polypeptide
GGGGGSGGGGGSGGGGGSGGGGGS

SEQ ID NO: 68 - protein, 230 aa, Artificial Sequence
  G3-Fc
GGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 242 aa, Artificial Sequence
  G4S3-Fc
GGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 49 aa, Artificial Sequence
  long incretin receptor ligand polypeptide with sortase tag
  MISC_FEATURE (2)..(2): Xaa denotes aib
  MISC_FEATURE (20)..(20): Xaa denotes aib
YXEGTFTSDYSIYLDKQAAXEFVAWLLAGGPSSGAPPPSKLPETGGSGS

SEQ ID NO: 71 - protein, 40 aa, Artificial Sequence
  short incretin receptor ligand polypeptide with sortase tag
  misc_feature (2)..(2): Xaa can be any naturally occurring amino acid
  misc_feature (20)..(20): Xaa can be any naturally occurring amino acid
YXEGTFTSDYSIYLDKQAAXEFVAWLLAGGGLPETGGSGS

SEQ ID NO: 72 - protein, 9 aa, Artificial Sequence
  sortase motif
LPETGGSGS

SEQ ID NO: 73 - protein, 5 aa, Artificial Sequence
  Synthetic peptide
  MISC_FEATURE (3)..(3): Xaa is an acidic amino acid such as Asp or Glu
LPXTG

SEQ ID NO: 74 - protein, 5 aa, Artificial Sequence
  Synthetic peptide
LPETG

SEQ ID NO: 75 - protein, 5 aa, Artificial Sequence
  Synethic peptide
  misc_feature (3)..(3): Xaa can be any naturally occurring amino acid
LPXTG

SEQ ID NO: 76 - protein, 29 aa, Homo sapiens
HSQGTFTSDYSKYLDSRRAQDFVQWLMNT

SEQ ID NO: 77 - protein, 42 aa, Homo sapiens
YAEGTFISDYSIAMDKIHQQDFVNWLLAQKGKKNDWKHNITQ

SEQ ID NO: 78 - protein, 10 aa, Artificial Sequence
  Synthetic peptide
GPSSGAPPPS

SEQ ID NO: 79 - protein, 11 aa, Artificial Sequence
  Synthetic peptide
  MISC_FEATURE (1)..(1): Xaa is any amino acid
XGPSSGAPPPS

SEQ ID NO: 80-81 - skipped (2 entries)

SEQ ID NO: 82 - protein, 6 aa, Artificial Sequence
  Synthetic peptide
GGSGGS

SEQ ID NO: 83 - protein, 9 aa, Artificial Sequence
  Synthetic peptide
GGSGGSGGS

SEQ ID NO: 84 - protein, 12 aa, Artificial Sequence
  Synthetic peptide
GGSGGSGGSGGS

SEQ ID NO: 85 - protein, 4 aa, Artificial Sequence
  Synthetic peptide
GGGG

SEQ ID NO: 86 - protein, 5 aa, Artificial Sequence
  Synthetic peptide
GGGGG

SEQ ID NO: 87 - protein, 6 aa, Artificial Sequence
  Synthetic peptide
GGGGGG

SEQ ID NO: 88 - protein, 4 aa, Artificial Sequence
  Synthetic peptide
GGGS

SEQ ID NO: 89 - protein, 8 aa, Artificial Sequence
  Synthetic peptide
GGGSGGGS

SEQ ID NO: 90 - protein, 5 aa, Artificial Sequence
  Synthetic peptide
GGGGS

SEQ ID NO: 91 - protein, 30 aa, Artificial Sequence
  Synthetic peptide
GGGGSGGGGSGGGGSGGGGSGGGGSGGGGS

SEQ ID NO: 92 - protein, 6 aa, Artificial Sequence
  Synthetic peptide
GGGGGS

SEQ ID NO: 93 - protein, 30 aa, Artificial Sequence
  Synthetic peptide
GGGGGSGGGGGSGGGGGSGGGGGSGGGGGS

SEQ ID NO: 94 - protein, 36 aa, Artificial Sequence
  Synthetic peptide
GGGGGSGGGGGSGGGGGSGGGGGSGGGGGSGGGGGS

SEQ ID NO: 95 - protein, 274 aa, Artificial Sequence
  Synthetic peptide
  MISC_FEATURE (2)..(2): Xaa is AIB
  MISC_FEATURE (20)..(20): Xaa is AIB
YXEGTFTSDYSIYLDKQAAXEFVAWLLAGGPSSGAPPPSKLPETGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 265 aa, Artificial Sequence
  Synthetic peptide
  MISC_FEATURE (2)..(2): Xaa is AIB
  MISC_FEATURE (20)..(20): Xaa is AIB
YXEGTFTSDYSIYLDKQAAXEFVAWLLAGGGLPETGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 286 aa, Artificial Sequence
  Synthetic peptide
  MISC_FEATURE (2)..(2): Xaa is AIB
  MISC_FEATURE (20)..(20): Xaa is AIB
YXEGTFTSDYSIYLDKQAAXEFVAWLLAGGPSSGAPPPSKLPETGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 277 aa, Artificial Sequence
  Synthetic peptide
  MISC_FEATURE (2)..(2): Xaa is AIB
  MISC_FEATURE (20)..(20): Xaa is AIB
YXEGTFTSDYSIYLDKQAAXEFVAWLLAGGGLPETGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK